(19) United States
(12) Patent Application Publication
(10) Pub. No.: US 2024/0398910 A1
(43) Pub. Date: Dec. 5, 2024

(54) METHODS AND COMPOSITIONS FOR THE TREATMENT OF CORONAVIRUS INFECTION, INCLUDING SARS-COV-2

(71) Applicant: Northwestern University, Evanston, IL (US)

(72) Inventors: Huiping Liu, Evanston, IL (US); Lamiaa Khamies Mohamed Ali El-Shennawy, Evanston, IL (US); Andrew Daniel Hoffman, Evanston, IL (US); Deyu Fang, Evanston, IL (US); Emma June Schuster, Evanston, IL (US); Nurmaa Khund Dashzeveg, Evanston, IL (US)

(21) Appl. No.: 18/658,779

(22) Filed: May 8, 2024

Related U.S. Application Data
(62) Division of application No. 17/457,405, filed on Dec. 2, 2021, now abandoned.
(60) Provisional application No. 63/238,075, filed on Aug. 27, 2021.
(60) Provisional application No. 63/120,444, filed on Dec. 2, 2020.

(51) Int. Cl. A61K 38/48; A61K 9/127; A61K 39/215; A61K 39/42; A61P 31/14; C07K 16/10

(52) U.S. Cl. CPC A61K 38/4813; A61K 9/127; A61K 39/215; A61K 39/42; A61P 31/14; C07K 16/1003

(57) ABSTRACT

Disclosed herein are exosomes, compositions, and methods for the treatment of subjects infected with, or at risk for infection with a coronavirus, such as SARS-COV-2, HCoV-NL63, or SARS-COV. The disclosed exosomes comprise ACE2 protein and typically display ACE2 protein on the exosome surface. In some embodiment, the exosomes optionally are loaded with one or more additional therapeutic agents for treating an infection by a coronavirus, such as remdesivir. In some embodiments, compositions comprising the exosomes are administered to a subject in need thereof, e.g., to a subject diagnosed with, or suspected of having a SARS-COV-2 infection, an HCoV-NL63 infection, or a SARS-COV infection. In some embodiments, administration is via inhalation. In some embodiments, administration is via injection.

### CROSS-REFERENCE TO RELATED APPLICATIONS

This application claims the benefit of U.S. Application 63/120,444, filed Dec. 2, 2020, and U.S. Application 63/238,075, filed Aug. 27, 2021, the entire contents of each are incorporated herein by reference in their entirety.

### SEQUENCE LISTING

A Sequence Listing accompanies this application and is submitted as an ASCII text file of the sequence listing named “702581_02071_ST25.txt” which is 77,750 bytes in size and was created on Dec. 2, 2021. The sequence listing is electronically submitted via EFS-Web with the application and is incorporated herein by reference in its entirety.

### FIELD

The field of the invention relates to compositions and methods for treating and/or preventing viral infections. In particular, the field of the invention relates to exosomes comprising the ACE2 protein, or viral spike protein (vaccine), or virus neutralizing immunoglobulin G (IgG) protein variants, or their encoding mRNAs/cDNAs, or anti-viral RNA analogs (e.g remdesivir), and uses thereof for treating and/or preventing infection by coronaviruses that utilize the ACE2 protein as a receptor for viral entry.

### BACKGROUND

In December 2019, an outbreak of a novel coronavirus, severe acute respiratory syndrome coronavirus 2 (SARS-COV-2), occurred in Wuhan, Hubei Province, People's Republic of China. This virus, which causes coronavirus disease 2019 (COVID-19) upon infection, has since become a global pandemic. Although patients initially present with fever with or without respiratory symptoms, various degrees of pulmonary abnormalities develop later in most patients, and these are typically observable via chest computed tomography (CT) imaging. Many patients only have a common, mild form of illness, but approximately 15% to 20% fall in the severe group, meaning they require assisted oxygenation as part of treatment. The severe group has a high mortality rate and is associated with older age, underlying diseases such as diabetes, and medical procedures (such as patients who were infected in a hospital setting while undergoing an operation for other indications). Although there have been several studies describing clinical features and characteristic radiographic findings (mainly chest CT scans), limited pathologic studies have been conducted on the basis of autopsies or biopsies. Some of the reasons for the lack of autopsies and biopsies include suddenness of the outbreak, vast patient volume in hospitals, shortage of health care personnel, and high rate of transmission, which makes invasive diagnostic procedures less of a clinical priority. Even so, information regarding the lung pathology and histology of subjects who have died of COVID-19 complication has been growing.

The COVID-19 pandemic has evolved to a worldwide crisis with more than 40 million cases and over 1 million deaths within 10 months. There are no specific therapeutics available to treat COVID-19 due to the SARS-COV-2 infection. Considering the SARS-COV-2 viral strain mutations, a neutralization approach to broad strains of SARS-COV-2 also is very desirable. Accordingly, there is a need in the art for methods and compositions to treat subjects suffering from SARS-COV-2 infection with attendant symptoms, at risk for infection, or infected with the SARS-COV-2 virus but not yet symptomatic.

### SUMMARY

Disclosed herein are exosomes, compositions comprising exosomes, and methods for the treatment of subjects infected with, or at risk for infection with SARS-COV-2, or other coronavirus such as HCoV-NL63 and SARS-COV. The disclosed exosomes comprise ACE2 protein or its mutant forms or viral antigen-specific IgGs, and typically display ACE2 protein or IgGs on the exosome outer surface. In some embodiment, the exosomes optionally are loaded with one or more additional therapeutic agents, such as remdesivir. In some embodiment, the exosomes optionally are loaded or engineered with viral spike protein or mRNAs/cDNAs. In some embodiments, compositions comprising the exosomes are administered to a subject in need thereof, e.g., to a subject diagnosed with, or suspected of having a SARS-COV-2 infection, an HCoV-NL63 infection or a SARS-CoV infection. In some embodiments, administration is via inhalation. In some embodiments, administration is via injection.

In some embodiments, an engineered exosome comprising one or more of ACE2 protein, or SARS-COV-2 specific IgG (IgG), or Coronavirus Spike, or a fragment or variant thereof is provided. In some embodiments, the ACE2 protein, IgG, or Spike represents at least about 10%, 20%, 30%, 40%, 50%, 60%, 70%, 80%, 90% or more of the total protein comprised by the engineered exosome. In some embodiments, the ACE2 protein, or IgG, or the fragment or variant thereof binds to the spike protein of a coronavirus selected from SARS-COV-2, SARS-COV, and HCoV-NL63. In some embodiments, the ACE2 protein comprises SEQ ID NO: 1. In some embodiments, the ACE2 protein has an amino acid sequence that is 95% identical to SEQ ID NO:1. In some embodiments, the ACE2 protein of SEQ ID NO: 1 comprises a point mutation comprising one or more of R621A, R697A, K702A, R705A, R708A, R710A, and R716A. In some embodiments, the ACE2 protein is resistant to cleavage by TMPRSS2. In some embodiments, the exosome comprises an additional active agent for treating and/or preventing infection by a coronavirus selected from SARS-COV-2, SARS-COV, and HCoV-NL63. In some embodiments, the additional active agent comprises remedsivir. In some embodiments, the exosome has an effective average diameter in the range of about 30-150 nm.

In some embodiments, a pharmaceutical composition comprising the engineered exosome is provided. In some embodiments, the composition is formulated as an aerosol, a lyophilized powder, or an emulsion. In some embodiments, the composition is formulated as a lyophilized powder. In some embodiments, the lyophilized powder is in a capsule or a cartridge. In some embodiments, the composition is formulated as an emulsion. In some embodiments, the isolated exosome formulated in an oily or aqueous vehicle. In some embodiments, the composition is formulated as an aerosol. In some embodiments, a system for delivering the aerosol formulated pharmaceutical composition is provided, comprising a device for administering the aerosol (e.g. an inhaler or a nebulizer).

In some embodiments, a method of treating and/or preventing SARS-COV-2 infection in a subject in need thereof, is provided. In some embodiments, the method includes administering to the subject an effective amount of the engineered exosomes of any of the aforementioned embodiments, and/or the pharmaceutical compositions of any of the aforementioned embodiments.

In some embodiments, a method of treating a subject diagnosed with a viral infection is provided. In some embodiments, the virus is utilizes the ACE2 protein as a receptor to infect cells of the subject, and the method comprising administering to the subject an effective amount of the engineered exosomes of any of the aforementioned embodiments, and/or the pharmaceutical compositions of any of the aforementioned embodiments. In some embodiments, the virus is SARS-COV or HCoV-NL63.

In some embodiments, an engineered exosome is provided, comprising one or more of: (a) SARS-COV-2 specific IgG (IgG), or a fragment or variant thereof; and (b) Coronavirus Spike, or a fragment or variant thereof.

In some embodiments, an engineered SARS-COV-2 specific IgG, or a fragment or variant thereof, with neutralization properties is provided to inhibit corona virus infections.

In some embodiments. a composition comprising a combination of SARS-COV-2 specific IgGs with optimal neutralization properties is provided to inhibit corona virus infections.

In some embodiments, a composition comprising (a) a combination of a SARS-COV-2 specific IgG, and (b) soluble or exosomal ACE2 is provided to neutralize corona virus infections.

### BRIEF DESCRIPTION OF THE DRAWINGS

FIG. 1a-i. Circulating evACE2 increased in the peripheral blood of COVID-19 patients. a. left panel—ACE2+ EVs detected in human plasma samples of sero-negative controls, acute phase, and convalescent COVID-19 patients. One-tail t test (*p=0.038, **p=0.0061 and **p=0.0016); right panel-exemplary protocol for the isolation or purification of ACE2 containing exosomes (extracellular vesicles), b. Representative microflow vesicolometry (MFV) plots with gated ACE2+EVs from sero-negative, acute phase and convalescent COVID-19 patients. c. MFV detection of circulating ACE2+ EVs with CD63+ EVs in human plasma of convalescent COVID-19 patient samples (CSB-029 and -023). Blue line is isotype IgG negative control. d. Flow profiles of ACE2 expression in HEK and HeLa parental control cells (con, light blue line, ACE2−) and with ACE2 overexpression (ACE2, green line). e. NanoSight NTA analysis of the sizes of HEK-derived ACE2− (ev1Con) and ACE2+ (ev1ACE2) and HeLa-derived ACE2-(ev2Con) and ACE2+ (cv2ACE2) f. Immunoblots of HEK and HeLa (ACE2− and ACE2+) EVs and cell lysates for ACE2, TSG101, CD63, CD81, GRP94 and loading control of the membrane proteins upon Ponceau staining. RIPA buffer and Bradford protein assay were used for cells/EVs lysis and protein measurement, respectively. g. Cryo-EM images of HEK-derived EVs, ACE2− (evCon, left) and ACE2+ (evACE2, right), stained with ACE2 (top) and CD81 (bottom). Scale bars=100 nm. h. Quantified counts of Apogee MFV-based total extracellular vesicles (EV) and ACE2+ EVs (N=2-3 independent experiments with n=6 technical replicates for total EV particles and n=3 technical replicates for ACE2+ counts). i. Overlay flow profiles of ACE2 positivity within CD63+ (left column) and CD81+ (right column) EVs isolated from HEK-ACE2 (top row) and HeLa-ACE2 (bottom row) cells, respectively (n=3 technical replicates).

FIG. 2a-h. Neutralization effects of evACE2 on RBD-binding and SARS-COV-2 variant infections. a. Schematic depiction of the cell based-neutralization assay. b. Representative flow profiles of RBD-A647 binding (16 and 3.3 nmol/L) to ACE2+ HEK-293 cells, inhibited by rhACE2 and ACE2+ EVs isolated from HEK-293 and HeLa cells whereas ACE2− EVs had no neutralization effects. c. IC50 of rhACE2 and ACE2 in the EVs from ACE2+ HEK (ev1ACE2) and HeLa (ev2ACE2) cells on 16 nM RBD-host cell binding (%). GraphPad Prism 9.1.2 was used to calculate the IC50. N≥2 experiments with two technical replicates for each. d. IC50 of evACE2, ev1 from HEK and ev2 from Hela cells, and rhACE2 neutralizing infections by wild-type (WT) S+ pseudotyped SARS-COV-2. GraphPad Prism 9.1.2 was used to calculate the IC50. N≥2 experiments with two technical replicates for each. e. IC50 (nM) of ACE2 in ev1ACE2 (HEK) and rhACE2 upon wild-type SARS-COV-2 infection. GraphPad Prism 9.1.2 was used to calculate the IC50 with three biological replicates. f. Distinct effects of ACE2+EVs and ACE2-control EVs on inhibiting Vero-6 cell death caused by SARS-COV-2. N=2 experiments with three biological replicates. g. The IC50 of ev1ACE2 (HEK) neutralizing infections by pseudotyped SARS-COV-2 expressing WT, B.1.1.7 (α) variant (red), B1.351 (β) variant (dark blue) and B.1.617.2 (δ) (light green) S protein. GraphPad Prism 9.1.2 was used to calculate the IC50. N=2 experiments with two technical replicates each. h. Effects of ev1ACE2 (HEK) on protecting Vero-6 cell viability against infections of SARS-COV-2 WT, B.1.1.7 (α) variant and B1.351 (β) variant (n=3 biological replicates).

FIG. 3a-f. evACE2 in patient plasma neutralizes SARS-COV-2. a. Schematic depiction of plasma EV ultracentrifugation and RBD-bead based depletion. b. Cryo-EM images of human EV pellets isolated from acute phase COVID-19 plasma (bar=100 nm). c. Immunoblots of plasma EV pellets (sero-negative and COVID-19 acute phase patients CBB-005 and -013) for ACE2 and loading control of protein staining with Ponceau). Laemmli buffer was used for lysis. d. ACE2+ EV pellets from acute phase patients 007, 008, 009, 012, and 013 blocked SARS-COV-2 infection-induced death of Vero6 cells whereas the sero-negative control and CBB-005 (no detectable ACE2) did not show neutralization effects. One-tail t test, *p=0.025, **p=0.006, **p=0.005 **p=0.004, and **p=0.003 shown as compared to sero-negative with n=2 biological replicates. e-f. Levels of ACE2+ EV counts (n=3 biological replicates). One-tail paired t test, *p-0.011 and **p=0.0063 (c) and altered neutralization effects on RBD-host cell binding (f) of the COVID-19 plasma EV pellets prior to and after RBD-bead depletion (convalescent phase CSB-012 and -024; acute phase CBB-008, 009, and 013). One-tail paired t test ****p=0.0001.

FIG. 4a-e. evACE2 inhibits SARS-COV-2 infection and inflammation in hACE2 transgenic mice. a. Probability of severe disease-free survival in B6.Cg-Tg (K18-ACE2) 2Prlmn/J (K18-hACE2) mice receiving SARS-COV-2 infection (10,000 pfu) and intranasal EVs (130 μg as measured on Nanodrop) per mouse. Log-rank (Mantel-Cox) and Gehan-Breslow-Wilcoxon tests ****p<0.0001. b. Viral loads in mouse lungs on day 5/6 after receiving SARS-COV-2 infection and administration of evCon (N=5 mice) or evACE2 (N=10 mice). T test-nonparametric-one tailed, *p=0.013. c. Representative H&E images of mouse lung sections at day 5 or 6 post virus inoculation and EV treatment (evCon and evACE2) intranasally. d-e: Acute and chronic inflammation scores (d), and alveolar hemorrhage and necrosis scores (e) in mouse lungs on day 5/6 after receiving evCon (N=5 mice) or evACE2 (N=7 mice). T test-nonparametric-one tailed, **p=0.005, **p=0.004 and ***p=0.000.

FIG. 5a-e. MFV analysis of evACE2 in COVID-19 plasma and SARS-COV-2 infected cells. a. EV staining protocol for Apogee-based microflow vesiclometry (MFV) on fluorescent and light scatter panels, SALS, MALS, and LALS. b. MFV of ACE2+ EV counts in human COVID-19 convalescent plasma of hospitalized and outpatients. One-tail t test, *p=0.0207. c. Light scatter channels (LALS and SALS) of patient specimen with or without CFSE staining showing the separate subset of small participle of free dye which can be used to gate certain antibody conjugates if not removed by centrifugation. In our ACE2 antibody and CD63 antibody staining, the antibody dye subsets were not present. d. MFV plots of unstained, IgG2a-AF488, and ACE2-AF488 stained patient plasma sample representatives of sero-negative control CSB-068, acute COVID-19 CBB-007, and convalescent CSB-023). The final counts are normalized after deducting the IgG isotype control noise for each patient plasma sample. e. Immunoblots of ACE2 and TSG101 of EVs ultracentrifuged (100,000 g×8 h at 4° C. after removing cell debris) from the culture supernatant of ACE2-overexpressing A549 cells with or without SARS-COV-2 infection (72 h). Equal volumes of supernatant were processed/loaded, and RIPA buffer was used to lyse isolated EVs.

FIG. 6a-e. Detection of evACE2 in human plasma and engineered cell culture. a. Exosome-enriched EV isolation protocol by ultracentrifugation. b. Schematic depiction of experiments evaluating soluble vs. vesicular ACE2. c. Immunoblots for ACE2, His-tag, and syntenin-1 in EVs isolations from the supernatant of HEK (con EV) and HEK ACE2 (ACE2 EV) with or without spiking of rhACE2 (2 μg). 100 and 200 ng of rhACE2 were directly loaded into the gels as positive controls for soluble ACE2. EVs were measured in PBS via Nanodrop and then lysed in urea buffer. d. Schematic depiction of Optiprep density gradient fractionation of ACE2 EVs isolated by ultracentrifugation. e. Immunoblots for ACE2, His-tag, HSP90, and CD81 of density gradient fractionated HEK ACE2 EVs. EVs were measured in PBS via Nanodrop and then lysed in urea buffer.

FIG. 7a-e. Analyses of evACE2 via Cryo-EM, MFV, ELISA and immune-blotting. a. Immuno-cyro-EM staining protocol with primary antibody and secondary antibody (conjugated with gold particles. b. Cryo-EM image of ACE2+ EVs, a pie graph (table), and plot of counted HEK-ACE2+ and ACE2− EVs. Two-tail t test (****p<0.0001). c. MFV profiles of exosomal CD63, CD81 and ACE2 expression in four cell line-derived EVs (HEK and HeLa with or without ACE2 expression). The final counts are normalized after deducting the IgG isotype control noise for each sample. N=2 independent experiments with n=3 technical replicates each. d. ELISA-based detection of ACE2 in ev1ACE2 (HEK) and ev2ACE2 (HeLa). EVs measured by Nanodrop in PBS. e. Immunoblots of ACE2 in purified ev1ACE2 (HEK) and ev2ACE2 (HeLa) in comparison to a serial dilution of rhACE2 (1-64 ng as measured by ELISA). EV proteins were measured in PBS by Nanodrop (27.3 and 87.5 μg for ev1 and ev2, respectively) and then lysed using RIPA buffer. The amount of ACE2 in EVs (ng ACE2/μg EV protein) is determined by densitometry of rhACE2 standards, within the range as measured by ELISA (FIG. 7d). X in a box indicating mean value and whisker lines extending to outliers (minimum and maximum) (n=5).

FIG. 8a-c. evACE2 blocks RBD binding to ACE2+HEK cells. a. Schematic of flow cytometry-based RBD binding to human host cells (ACE2+ HEK) for neutralization effect analysis. b. Flow plots of ACE2+ and ACE2− HEK cells in the absence and presence of AF647-conjugated RBD binding with minimal binding to a mock control of the RBD probe. c. Histogram bars of quantified RBD-neutralization by evACE2 (ev1 from HEK and ev2 from HeLa) in a dose dependent manner in comparison to ACE2− parental cell derived control EVs (ev1Con and ev2Con from HEK and HeLa, respectively (n=2 technical replicates). One-way ANOVA followed by Tukey's multiple comparisons (**p=0.0016, **p=0.0011, **p=0.0058, ***p=0.0003 ****p=0.0000 (<0.0001) and ns=non-significant compared to evCon or PBS).

FIG. 9a-i. ACE2+ EVs block pseudotyped and authentic SARS-COV-2 infections to host cells. a. Schematic of the three vectors for SARS-COV-2 S+pseudovirus production. b. Pseudovirus infection with ACE2+ HeLa cells and subsequent infectivity analyses, including Cherry positive cells under fluorescent microscopy, MFV, and luciferase activity assays. c. Fluorescent images of Cherry+ red cells as pseudovirus-infected cells. d-e. Flow plots (d) and bar graph (e) of pseudovirus-infected HeLa-ACE2 cells, detected with Cherry reporter expression which was inhibited by rhACE2 and ACE2+ EVs, but not ACE2− EVs. One-way ANOVA followed by Tukey's multiple comparisons (*p=0.026, **p=0.005, **p=0.002, ***p=0.0007, ****p=0.0004 and ns=non-significant compared to PBS+Spike). N=2 experiments with two technical replicates each. f. Luciferase-based viral infectivity between ACE2-control and ACE2+ HEK cells (left panel) and between ACE2-control and ACE2+ HeLa cells (right panel), 72 hr after incubation with bald (S−) or S+ pseudotyped viruses in the absence and presence of ACE2+ or ACE2− EVs (****p<0.0001). One-tail t test, **p=0.002, **p=0.003, **p=0.004 **p=0.005 and ****p=0.000 (<0.0001) compared to respective no virus. N=2 experiments with two technical replicates each. g-h. Luciferase-based SARS-COV-2 S+ pseudotype infectivity of HeLa-ACE2 cells in the presence of ACE2− and ACE2+ EV2s from both HEK (g) and HeLa (h) cells (n=2 technical replicates). One-tail t test, **p=0.004, **p-0.002, ***p-0.0004, ***p=0.0002 and ***p=0.0001 compared to respective ACE2− EVs). i. SARS-COV-2 viral loads of Vero-6 host cells after viral infections in the presence of 10 μg EV1/EV2 protein measured via Nanodrop (control and ACE2, ˜1 ng as determined by ELISA), rhACE2 (128 ng as determined by ELISA), and vehicle (n=3 biological replicates). One-tail t test, *p=0.026, *p=0.011 and ***p=0.0007.

FIG. 10a-h. Patient plasma analysis and mouse biodistribution of therapeutic evACE2. a. RBD-IgG levels detected in human plasma of sero-negative, acute phase, and convalescent COVID-19. One-tail t test. b. Negative correlation between COVID-19 plasma RBD-IgG levels and the RBD-host cell binding. Adjusted R square 0.623 with P=3.58E-07. c. Negative association of RBD binding to host cells with the integrated RBD-IgG and ACE2+ EV levels in the convalescent COVID-19 patient plasma (N=30). d. RBD-IgG levels in plasma samples of sero-negative control and acute and convalescent patients. e. Immunoblots of plasma EV pellets for ACE2 and TSG-101, isolated from NWL-001 (lung cancer patient), and a COVID-19 convalescent patient CSB-012. f. Minimal or residual RBD-IgG in plasma EV pellet prior to and after RBD bead depletion compared to crude plasma of convalescent COVID-19 patients (CSB-012 and -024) (n=4). g. Fluorescence images of dissected lungs of B6 mice at 24 h post intranasal delivery of PBS, PKH-67 dilutant buffer control without EVs, and PKH-67-labeled evACE2 (130 μg/mouse). h. Levels of fluorescent signals (evACE2 biodistribution) to the various organs, with the most majority to the lungs and not significant distribution to brain, heart, liver, kidney and spleen (N=3-4 biological replicates). Non-parametric one-tail t test (*p=0.033 and ns=non-significant). Whiskers represent minimum to maximum showing all points.

FIG. 11 is a table showing Go Annotation of PBD-bead precipitated EVs and proteins from human plasma, including pre-COVID-19 (NWL-001 and NWL-004 lung cancer patients), convalescent COVID-19 patients (CSB-012 and CSB-024), and proteomic profiles of positive controls of rhACE2 and evACE2 (HEK).

FIG. 12 is a table showing the antibody conditions for immunoblotting.

FIG. 13 is a table showing the depletion conditions with RBD conjugation and beads.

FIG. 14 is a table showing histopathological grading.

FIG. 15a-f Neutralizing IgG accumulated in the plasma exosomes of convalescent COVID-19 patients. a. Protocol of ultracentrifugation to deplete exosomes. b. RBD binding to ACE2+ HEK-293 cells prior to and after exosome depletion from four convalescent plasma specimens (012, 016, 018, and 026). c-d. Spike-IgG (c) and RBD-IgG (d) measurement in the plasma with 2-18 hr ultracentriugation. e. Immunoblotting of human IgG (H+L) in crude plasma, 8 h ultracentrifugation pellet, and RBD-beads collected of COVID-19 convalescent patient plasma. f. Microflow vesiclometry of human IgG (H+L) in patient plasma exosomes or vesicles.

FIG. 16. Sequence of vector Abvec hIgG1 empty.

FIG. 17a-g. Detection of ACE2+ exosomes in human plasma of pre-COVID-19 and COVID-19 (CSB) patients. a-b. MFV profiles and counts of ACE2+ exosomes in patient plasma, pre-COVID-19 (NWL-01, -04) and COVID-19 plasma (CSB-012, 024) c. Positive correlation with COVID-19 plasma RBD-IgG levels and the neutralization effects on RBD-host cell binding. d. Absence of RBD-IgG in the pre-COVID-19 (NWL) plasma versus various levels in the COVID-19 plasma. e. Schematic of exosome enrichment via ultracentrifugation and depletion of ACE2+ exosomes by RBD-beads. f. Levels of ACE2+ exosome counts and RBD-IgG in the plasma pellet prior to and after RBD-bead depletion. g. RBD-bead depletion compromised the neutralization of pellet exosomes and restored RBD binding to host cells.

FIG. 18a-c. Silver-stained gel and immunoblotting of RBD bead-bound proteins. a. Schematic of the protocols of exosome enriched pellet collection and RBD-bead mediated collection of proteins and exosomes; b. Left panel: protein analysis of RBD beads bound with COVID-19 plasma on SDS-PAGE gel with silver staining. The beads were resuspended in protein denaturing gel buffer and boiled for 5 minutes. Right panel: HEK-ACE2 exosome and rhACE2 were western blotted for ACE2 detection c. Siver stained gel image of RBD beads proteins collected from pre-COVID-19 plasma and HEK-ACE2 exosomes (Left panel) and western blotting for human IgG (H and L chains) pulled down by RBD-beads.

FIG. 19a-c. Single-cell RNA seq of sorted RBD-specific memory B cells from convalescent COVID-19 patients for IgG cloning. b. Flow profiles of sorted B cells (CD19+IgM-CD27-med CD38-RBD+) specific to RBD. b. The age and B cell # sorted from each COVID-19 convalescent patient. c. Association analyses between RBD binding to cells/ACE2 and the IgG specific to RBD or Spike.

FIG. 20a-b. Structure-based quality prediction of RBD-specific IgG clones. a. Representative structure models of S-RBD, antibody (IgG)-H, and Antibody (IgG)-L interactions. b. Table of 16 antibody H/L clonotypes with scores of 4 listed models and overall scores.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an exosome” should be interpreted to mean “one or more exosomes.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

In some embodiments, a subject may be in need of treatment, for example, treatment may include administering a therapeutic amount of one or more agents that inhibits, alleviates, or reduces the signs or symptoms of viral infection, such as infection from SARS-COV-2.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

As used herein “control,” as in “control subject” or “control sample” has its ordinary meaning in the art, and refers to a sample, or a subject, that is appropriately matched to the test subject or test sample and is treated or not treated as appropriate.

As used herein, the term Severe Acute Respiratory Syndrome Coronavirus 2 (SARS-CoV-2) refers to an enveloped, non-segmented, positive sense RNA virus that is included in the sarbecovirus, ortho corona virinae subfamily which is broadly distributed in humans and other mammals. Its diameter is about 65-125 nm, containing single strands of RNA and provided with crown-like spikes on the outer surface. A nucleic acid sample isolation from pneumonia patients who were some of the workers in the Wuhan seafood market found that strains of SARS-COV-2 had a length of 29.9 kb. Structurally, SARS-COV-2 has four main proteins including spike(S) glycoprotein, small envelope (E) glycoprotein, membrane (M) glycoprotein, and nucleocapsid (N) protein, and also several accessory proteins. The S, E, and M proteins together create the viral envelope, while the N protein holds the RNA genome. The name “coronavirus” is derived from the Latin word “corona” meaning crown or halo, and refers to the characteristic appearance of the virus under an electron microscopy, where the virus includes a fringe of large, bulbous surface projections creating an image reminiscent of a crown or halo. This coronal morphology is created by the viral spike protein(S), which is present on the surface of the virus. The spike or S glycoprotein is a transmembrane protein with a molecular weight of about 150 kDa found on the outer portion of the virus and is 1273 amino acids in length. The S protein mediates viral entry into host cells by first binding to a host receptor, the angiotensin 1 converting enzyme 2 (ACE2), through the receptor-binding domain (RBD) in the S1 subunit and then fusing the viral and host membranes through the S2 subunit. ACE2 is widely found in different organs such as the lung, kidney, heart, and endothelial tissue. Therefore, patients who are infected with this virus not only experience respiratory problems such as pneumonia leading to Acute Respiratory Distress Syndrome (ARDS), but also experience disorders of the heart, kidneys, and digestive tract.

As used herein the term “exosome” is used interchangeably with “extracellular vesicle,” and refers to a cell-derived small (between 20-400 nm in diameter, more preferably 30-150 nm in diameter) vesicle comprising a membrane that encloses an internal space, and which is generated from the cell by direct plasma membrane budding or by fusion of the late endosome with the plasma membrane. Exosomes (extracellular vesicle), transport signaling proteins, nucleic acids, and lipids among cells. They are actively secreted by almost all types of cells, exist in body fluids, and circulate in the blood.

In various embodiments, the exosome membrane comprises an interior surface and an exterior surface and encloses an internal space. In some embodiments, the exosome further comprises a payload. In some embodiments, the payload is enclosed within the internal space. In some embodiments, the payload is displayed on the external surface of the exosome. In some embodiments, the payload spans the membrane of the exosome. In various embodiments, the payload comprises one or more of a decoy molecule, a therapeutic agent, a targeting moiety, and an imaging protein. In some embodiment the payload comprises one or more of a protein, a polynucleotide (e.g., a nucleic acid, RNA, or DNA), carbohydrates, sugars (e.g., a simple sugar, polysaccharide, or glycan), lipids, small molecules, and/or combinations thereof. The exosome can be derived from a producer cell (e.g., a cell engineered to overexpress a protein of interest, e.g., a payload, or other molecule of interest for incorporation into the exosome) or from body fluids (e.g., plasma), and isolated from the producer cell (culture supernatants) and body fluids based on its size, density, biochemical parameters, or a combination thereof.

In various embodiments, the exosome is a membrane-bound vesicle that has a smaller diameter than the cell from which it is derived. In some embodiments, the exosome has a longest dimension between about 20-1000 nm, such as between about 20-100 nm, 20-200 nm, 20-300 nm, 20-400 nm, 20-500 nm, 20-600 nm, 20-700 nm, 20-800 nm, 20-900 nm, 30-100 nm, 30-200 nm, 30-300 nm, 30-400 nm, 30-500 nm, 30-600 nm, 30-700 nm, 30-800 nm, 30-900 nm, 40-100 nm, 40-200 nm, 40-300 nm, 40-400 nm, 40-500 nm, 40-600 nm, 40-700 nm, 40-800 nm, 40-900 nm, 50-150 nm, 50-500 nm, 50-750 nm, 100-200 nm, 100-500 nm, or 500-1000 nm. In some embodiments, the exosome has a longest dimension between about 10-50 to about 100-300, or between about 30-150, 30-250, 30-350, 30-450.

As used herein, the term “producer cell” refers to any cell from which an exosome can be isolated. A producer cell is a cell which serves as a source for the exosome. A producer cell can share a protein, lipid, sugar, or nucleic acid component with the exosome. In some embodiments, the producer cell is a modified (engineered) or synthetic cell. In some embodiments, the producer cell is a cultured or isolated cell. In certain embodiments, the producer cell is a cell line. By way of example but not by way of limitation, in some embodiments, the producer cell comprises a HEK 293 cell, a HeLa cell, a mesenchymal stem cell, a dendritic cell, a B lymphocyte, or a bone marrow-derived stroma cell. In some embodiments, the producer cell is a mammalian cell and is engineered to express, or overexpress, ACE2 protein, SARS-COV-2 spike protein, or a SARS-COV-2 recognizing Ig protein, as well as mutants or variants thereof. In some embodiments, the ACE2 protein comprises SEQ ID NO: 1, a variant thereof or a fragment thereof.

A “therapeutic agent” or “therapeutic molecule” includes a compound or molecule that, when present in an effective amount, produces a desired therapeutic effect, pharmacologic and/or physiologic effect on a subject in need thereof. It includes any compound, e.g., a small molecule drug, or a biologic (e.g., a polypeptide drug or a nucleic acid drug) that when administered to a subject has a measurable or conveyable effect on the subject, e.g., it alleviates or decreases a symptom of a disease, disorder or condition. By way of example but not by way of limitation, a therapeutic agent includes exosomes of the present disclosure expressing a decoy receptor such as ACE2, viral spike protein, a virus-neutralizing antibody (or Ig), with or without its transmembrane domain and cytoplasmic tail, or a fragment or variant thereof, and optionally, one or more additional therapeutic agents such as remdesivir and mRNA/cDNAs of the listed proteins.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired extracellular vesicles, that have undergone one or more processes of purification, e.g., a selection or an enrichment of the desired extracellular vesicle preparation. In some embodiments, isolating or purifying as used herein is the process of removing, partially removing (e.g. a fraction) of the extracellular vesicles from a sample containing producer cells. In some embodiments, an isolated extracellular vesicle composition has no detectable undesired activity or, alternatively, the level or amount of the undesired activity is at or below an acceptable level or amount. In other embodiments, an isolated extracellular vesicle composition has an amount and/or concentration of desired extracellular vesicles at or above an acceptable amount and/or concentration. In other embodiments, the isolated extracellular vesicle composition is enriched as compared to the starting material (e.g. producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some embodiments, isolated extracellular vesicle preparations are substantially free of residual biological products. In some embodiments, the isolated extracellular vesicle preparations are 100% free, 99% free, 98% free, 97% free, 96% free, or 95% free of any contaminating biological matter. Residual biological products can include abiotic materials (including chemicals) or unwanted nucleic acids, proteins, lipids, or metabolites. Substantially free of residual biological products can also mean that the extracellular vesicle composition contains no detectable producer cells and that only extracellular vesicles are detectable.

### Polynucleotides

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1 (3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences-a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26 (3/4):227-259; and Owczarzy et al., 2008, Biochemistry, 47: 5336-5353, which are incorporated herein by reference).

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered expression templates include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

### Polypeptides

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asp, Gln, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Lcu, Ile |
| Phe | His, Met, Leu, Trp, Tyr |
| Ser | Cys, Thr |
| Thr | Ser, Val |
| Trp | Phe, Tyr |
| Tyr | His, Phe, Tyr |
| Val | Ile, Leu, Thr |

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. A “variant” of a reference polypeptide sequence may include a conservative or non-conservative amino acid substitution relative to the reference polypeptide sequence.

The disclosed peptides may include an N-terminal esterification (e.g., a phosphoester modification) or a pegylation modification, for example, to enhance plasma stability (e.g. resistance to exopeptidases) and/or to reduce immunogenicity.

A “deletion” refers to a change in a reference amino acid sequence (e.g., SEQ ID NO:1 (human ACE2 polypeptide sequence) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence (e.g., a fusion partner) that extends the half-life of the fusion polypeptide in the tissue of interest, such as serum, plasma. A “variant” of a reference polypeptide sequence may include a fusion polypeptide comprising the reference polypeptide.

A “fragment” is a portion of an amino acid sequence which is identical in sequence to but shorter in length than a reference sequence (e.g., SEQ ID NO:1). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

### ACE2

As used herein, the term “ACE2” refers to the angiotensin 1 converting enzyme 2, which is a member of the family of dipeptidyl carboxydipeptidases. ACE2 has considerable homology to human angiotensin 1 converting enzyme. This secreted protein catalyzes the cleavage of angiotensin I into angiotensin 1-9, and angiotensin II into the vasodilator angiotensin 1-7. The organ- and cell-specific expression of this gene suggests that it may play a role in the regulation of cardiovascular and renal function, as well as fertility. In addition, the encoded protein is a functional receptor for the spike glycoprotein of the human coronavirus HCoV-NL63 and the human severe acute respiratory syndrome coronaviruses, SARS-COV and SARS-COV-2 (COVID-19 virus). The sequence of human ACE2 is shown below as SEQ ID NO: 1 (GenBank: BAB40370.1; Accession No. AB046569.1) or UniProtKB-Q9BYF1 (ACE2_HUMAN) with the amino acids 1-17 as a signal peptide. The mature ACE2 proteins are either full length amino acids 18-805 or processed amino acids 18-708 (extracellular domains) after protein cleavage.

| SEQ ID NO: 1 | |
|---|---|
| 1 | msssswllls lvavtaaqst ieeqaktfld kfnheaedlf yqsslaswny ntniteenvq |
| 61 | nmnnagdkws aflkeqstla qmyplqeiqn ltvklqlqal qqngssvlse dkskrlntil |
| 121 | ntmstiystg kvcnpdnpqe clllepglne imansldyne rlwaweswrs evgkqlrply |
| 181 | eeyvvlknem aranhyedyg dywrgdyevn gvdgydysrg qliedvehtf eeikplyehl |
| 241 | hayvraklmn aypsyispig clpahllgdm wgrfwtnlys ltvpfgqkpn idvtdamvdq |
| 301 | awdaqrifke aekffvsvgl pnmtqgfwen smltdpgnvq kavchptawd lgkgdfrilm |
| 361 | ctkvtmddfl tahhemghiq ydmayaaqpf llrnganegf heavgeimsl saatpkhlks |
| 421 | igllspdfqe dneteinfll kqaltivgtl pftymlekwr wmvfkgeipk dqwmkkwwem |
| 481 | kreivgvvep vphdetycdp aslfhvsndy sfiryytrtl yqfqfqealc qaakhegplh |
| 541 | kcdisnstea gqklfnmlrl gksepwtlal envvgaknmn vrpllnyfep lftwlkdqnk |
| 601 | nsfvgwstdw spyadqsikv rislksalgd rayewndnem ylfrssvaya mrqyflkvkn |
| 661 | qmilfgeedv rvanlkpris fnffvtapkn vsdiiprtev ekairmsrsr indafrlndn |
| 721 | sleflgiqpt lgppnqppvs iwlivfgvvm gvivvgivil iftgirdrkk knkarsgenp |
| 781 | yasidiskge nnpgfqntdd vqtsf |

Variants of ACE2 may be used in the compositions and methods disclosed herein. By way of example but not by way of limitation, an ACE2 variant comprising substitutions (RK7A), including R621A, R697A, K702A, R705A, R708A, R710A, and R716A may be incorporated into the exosomes and therapeutic compositions of the present disclosure. A sequence of the fluorescent protein eGFP may also be fused to the C-terminus of the ACE2 in order to label engineered cells and exosomes.

### Viral Spike

As used herein, the term “spike” refers to a spike viral envelope protein that utilizes ACE2 for cellular entry. One exemplary spike protein is the SARS-COV-2 spike(S) glycoprotein, which is a viral surface antigen of transmembrane glycoproteins responsible for host cell attachment and membrane fusion upon binding to ACE2. The spike protein is known to elicit immune responses so the exosomes comprising spike protein or mRNAs can be used in vaccine development and applications. The SARS-COV-2 spike protein is highly homologous to other betacoronavirus spike proteins, such as SARS-COV and HCoV-NL63, which might also be overexpressed and presented in the exosomes. The sequence of SARS-COV-2 spike(S) is shown below as SEQ ID NO: 2 (NCBI reference sequence: NC_045512.2) and UniProtKB-P0DTC2 (SPIKE_SARS2).

| | |
|---|---|
| 1 | mfvflvllpl vssqcvnitt rtqlppaytn sftrgvyypd kvfrssvlhs |
| 51 | tqdlflpffs nvtwfhaihv sgtngtkrfd npvlpfndgv yfasteksni |
| 101 | irgwifgttl dsktqsliiv nnatnvvikv cefqfcndpf lgvyyhknnk |
| 151 | swmesefrvy ssannctfey vsqpflmdle gkqgnfknlr efvfknidgy |
| 201 | fkiyskhtpi nlvrdlpqgf saleplvdlp iginitrfqt llalhrsylt |
| 251 | pgdsssgwta gaaayyvgyl qprtfllkyn engtitdavd caldplsetk |
| 301 | ctlksftvek giyqtsnfrv qptesivrfp nitnlcpfge vfnatrfasv |
| 351 | yawnrkrisn cvadysvlyn sasfstfkcy gvsptklndl cftnvyadsf |
| 401 | virgdevrqi apgqtgkiad ynyklpddft gcviawnsnn ldskvggnyn |
| 451 | ylyrlfrksn lkpferdist eiyqagstpc ngvegfncyf plqsygfqpt |
| 501 | ngvgyqpyrv vvlsfellha patvcgpkks tnlvknkcvn fnfngitgtg |
| 551 | vltesnkkfl pfqqfgrdia dttdavrdpq tleilditpc sfggvsvitp |
| 601 | gtntsnqvav lyqdvnctev pvaihadqlt ptwrvystgs nvfqtragcl |
| 651 | igaehvnnsy ecdipigagi casyqtqtns prrarsvasq siiaytmslg |
| 701 | aensvaysnn siaiptnfti svtteilpvs mtktsvdctm yicgdstecs |
| 751 | nlllqygsfc tqlnraltgi aveqdkntqe vfaqvkqiyk tppikdfggf |
| 801 | nfsqilpdps kpskrsfied lifnkvtlad agfikqygdc lgdiaardli |
| 851 | caqkfngltv lpplltdemi aqytsallag titsgwtfga gaalqipfam |
| 901 | qmayrfngig vtqnvlyenq klianqfnsa igkiqdslss tasalgklqd |
| 951 | vvnqnaqaln tlvkqlssnf gaissvlndi lsrldkveae vqidrlitgr |
| 1001 | lqslqtyvtq qliraaeira sanlaatkms ecvlgqskrv dfcgkgyhlm |
| 1051 | sfpqsaphgv vflhvtyvpa qeknfttapa ichdgkahfp regvfvsngt |
| 1101 | hwfvtqrnfy epqiittdnt fvsgncdvvi givnntvydp lqpeldsfke |
| 1151 | eldkyfknht spdvdlgdis ginasvvniq keidrlneva knlneslidl |
| 1201 | qelgkyeqyi kwpwyiwlgf iagliaivmv timlccmtsc csclkgccsc |
| 1251 | gscckfdedd sepvlkgvkl hyt |

Variants of spike may be used in the compositions and methods disclosed herein. By way of example but not by way of limitation, a spike variant comprising a substitution of D614G or D614A maybe be incorporated into the exosomes and therapeutic compositions of the present disclosure.

### Viral Antigen-Specific IgG

As used herein, the term “SARS-COV-2 IgG” refers to SARS-COV-2 antigen-specific IgG, which is a tetramer of two heavy chains and two light chains sequenced from antigen-baited human memory B lymphocytes of convalescent COVID-19 patients via single-cell RNA sequencing using 10× genomics kit. The SARS-COV-2 spike antigen receptor-binding region (RBD) (R319-F541), was used for baiting and sorting of RBD-specific B cells (CD19⁺IgM⁻ CD27^{med}CD38⁻RBD⁺) from the convalescent blood. The synthesized RBD protein with a C-terminal his-tag was conjugated with biotin and then bound to streptavidin-A647 fluorophore for flow sorting of RBD-specific B cells. Sequences of IgG antibodies were retrieved from these B cells, including both heavy (H) and light (L) chain sequences disclosed herein.

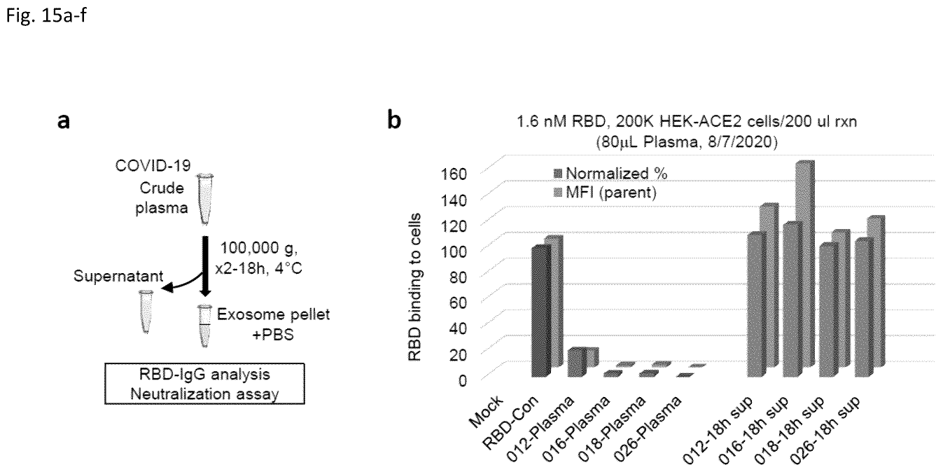

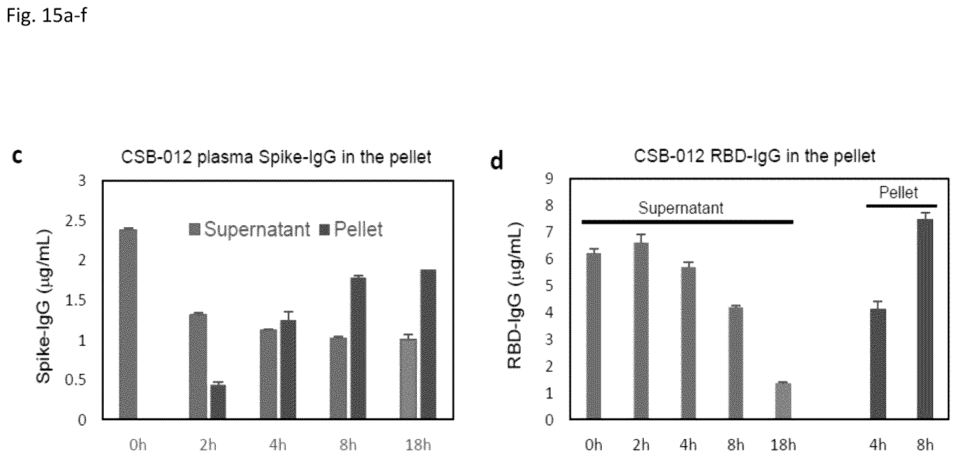

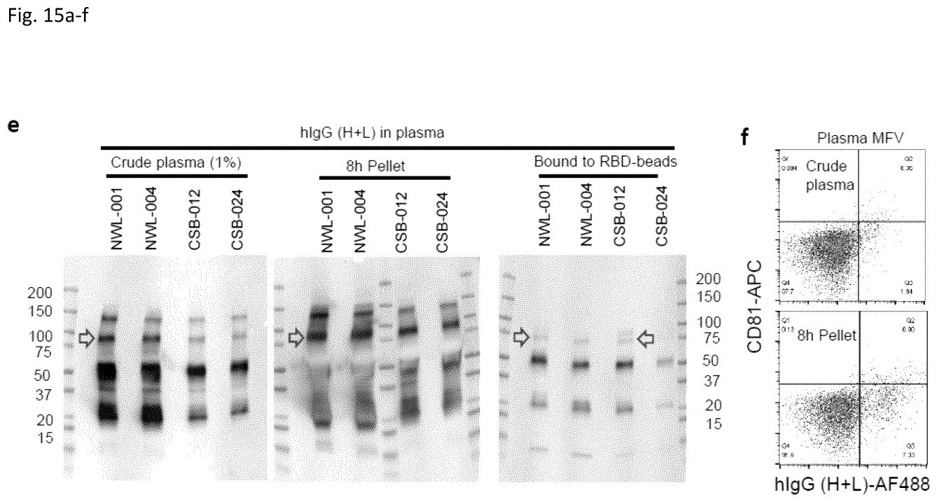

SARS-COV-2 spike (RBD)-specific neutralizing IgG (H+L) is detectable on the surface of exosomes by micro flow vesiclometry and by western blotting (see FIG. 15, e and f). The human IgG heavy chain determines the subclass of IgG (IgG1, IgG2, IgG3, and IgG4) and can be cloned with or without a transmembrane domain (TMD) which is used to locate IgG to the exosome membrane for optimal neutralization functions. The human Ig light chain is a sequence of either kappa (IgK) or lambda (IgL). Paired H and L sequences from a single B cell are cloned into individual IgG expressing vectors for antibody generation in HEK-293 cells or other mammalian cells. Except for one B cell showing one IgG heavy chain sequence and two distinct IgK light chain sequences, the rest of the sequenced B cells contain one heavy chain sequence (IgG) and one light chain sequence (IgK or IgL). See Table 1, below.

| Ig heavy chain protein sequence hIgGl-0001 (1-539) with TMD(486-511 | |
|---|---|
| in bold) | |
| 1 | mgwsciilfl vatatgvhse vqlvqsgaev kkpgasvkvs ckasgytftd |
| 51 | yyihwvrqap gqglewmgwi npisggtnya qkfqgrvtmt rdtsvttfym |
| 101 | elswltsdds avyycarcpf fysetsgy fdywgqgtlv tvssastkgpsv |
| 151 | fplapsskst sggtaalgcl vkdyfpepvt vswnsgalts gvhtfpavlq |
| 201 | ssglyslssv vtvpssslgt qtyicnvnhk psntkvdkkv epkscdktht |
| 251 | cppcpapell ggpsvflfpp kpkdtlmisr tpevtcvvvd vshedpevkf |
| 301 | nwyvdgvevh naktkpreeq ynstyrvvsv ltvlhqdwln gkeykckvsn |
| 351 | kalpapiekt iskakgqpre pqvytlppsr deltknqvsl tclvkgfyps |
| 401 | diavewesng qpennykttp pvldsdgsff lyskltvdks rwqqgnvfsc |
| 451 | svmhealhnh ytqkslslsp qleescaeaq dgeldglwtt itifitlfll |
| 501 | svcysatvtf fkvkwifssv vdlkqtiipd yrnmigqga |
| Ig heavy chain protein sequence hIgG 1-0001 (1-472) without TMD | |
| 1 | mgwsciilfl vatatgvhse vqlvqsgaev kkpgasvkvs ckasgytftd |
| 51 | yyihwvrqap gqglewmgwi npisggtnya qkfqgrvtmt rdtsvttfym |
| 101 | elswltsdds avyycarcpf fysetsgy fdywgqgtlv tvssastkgpsv |
| 151 | fplapsskst sggtaalgcl vkdyfpepvt vswnsgalts gvhtfpavlq |
| 201 | ssglyslssv vtvpssslgt qtyicnvnhk psntkvdkkv epkscdktht |
| 251 | cppcpapell ggpsvflfpp kpkdtlmisr tpevtcvvvd vshedpevkf |
| 301 | nwyvdgvevh naktkpreeq ynstyrvvsv ltvlhqdwln gkeykckvsn |
| 351 | kalpapiekt iskakgqpre pqvytlppsr deltknqvsl tclvkgfyps |
| 401 | diavewesng qpennykttp pvldsdgsff lyskltvdks rwqqgnvfsc |
| 451 | svmhealhnh ytqkslslsp gk |
| Ig light chain protein sequence hIgL-0002 (1-235) | |
| 1 | mgwsciilfl vatatgswaq saltqpasvs gspgqsitis ctgtssdvgg |
| 51 | ynyvswfqqh pgkapklmiy evstrpsgvs nrfsgsksgn tasltisglq |
| 101 | aedeadyycs sytssntyvf gtgtqvtvlg qpkanptvtl fppsseelqa |
| 151 | nkatlvclis dfypgavtva wkadsspvka gvetttpskq snnkyaassy |
| 201 | lsltpeqwks hrsyscqvth egstvektva ptecs |

In some embodiments, the engineered IgG heavy chain includes the shared constant regions as well as the TMD and a cytoplasmic tail to produce a polypeptide of 539 amino acids. In some embodiments, the engineered IgG heavy chain only includes the shared constant regions without a TMD and a cytoplasmic tail to produce a polypeptide of 472 amino acids. The paired 25 sets of IgG heavy and light chain insert sequences are the cDNA nucleotides encoding the variable regions as shown below (each set has one heavy chain followed by one light chain except for one set 0051 heavy chain IgG1 paired with two light chains 0052 IgK and 0053 IgK).

TABLE 1
IgG sequences

| Name | Sequence |
|---|---|
| 0001-IGG1 | ATCCTTTTTCTAGTAGCAACTGCAACCGGTGTACA |
| | TTCCGAGGTGCAGCTGGTGCAGTCTGGGGCTGAGG |
| | TGAAGAAGCCTGGGGCCTCAGTGAAGGTCTCCTGC |
| | AAGGCTTCTGGATACACCTTCACCGACTACTATAT |
| | ACACTGGGTGCGACAGGCCCCTGGACAAGGGCTTG |
| | AGTGGATGGGATGGATCAACCCTATAAGTGGTGGC |
| | ACAAACTATGCACAGAAGTTTCAGGGCAGGGTCAC |
| | CATGACCAGGGACACGTCCGTCACCACTTTTTACA |
| | TGGAGCTGAGCTGGCTGACATCTGACGACTCGGCC |
| | GTATATTACTGTGCGAGATGCCCGTTCTTTTACTC |
| | TGAAACTAGTGGTTATTTCGACTACTGGGGCCAGG |
| | GAACCCTGGTCACCGTCTCCTCAGCGTCGACCAAG |
| | GGCCCATCGGTCTTCC |
| 0002-IGL1 | ATCCTTTTTCTAGTAGCAACTGCAACCGGTTCCTG |
| | GGCCCAGTCTGCCCTGACTCAGCCTGCCTCCGTGT |
| | CTGGGTCTCCTGGACAGTCGATTACCATCTCCTGC |
| | ACTGGAACCAGCAGTGACGTTGGTGGTTATAACTA |
| | TGTCTCCTGGTTCCAACAGCACCCAGGCAAAGCCC |
| | CCAAACTCATGATTTATGAGGTCAGTACTCGGCCC |
| | TCAGGGGTTTCTAATCGCTTCTCTGGCTCCAAGTC |
| | TGGCAACACGGCCTCCCTGACCATCTCTGGGCTCC |
| | AGGCTGAGGACGAGGCTGATTATTACTGCAGCTCA |
| | TATACAAGCAGCAACACTTATGTCTTCGGAACTGG |
| | GACCCAGGTCACCGTCCTAGGTCAGCCCAAGGCCA |
| | ACCCCACTGTCACTCTGTTCCCACCCTCGAGTGAG |
| | GAGCTTCAAGCCAACA |
| 0007-IGG1 | ATCCTTTTTCTAGTAGCAACTGCAACCGGTGTACA |
| | TTCTGAGGTGCAGCTGGTGGAGTCGGGGGGAGGCT |
| | TGGTCCAGCCTGGGGGGTCCCTGAGACTCTCCTGT |
| | GCAGCCTCTGGATTCACCTTCAGTAGCTACGACAT |
| | GCACTGGGTCCGCCAAGCTACAGGAAAAGGTCTGG |
| | AGTGGGTCTCCACTATTGGTACTG |
| | CTGGTGACACATATTATTTAGGTTCCGTGAAGGGC |
| | CGATTTACCTTCTCCAGAGAAAATGCCAAGAACTC |
| | CTTGTATCTTCAAATGAACAGCCTGAGAGCCGGGG |
| | ACACGGCTGTGTATTACTGTGCAAGAGCGAAGTAC |
| | TATGATAGTAGTGGTTATTATCACTACACGCCCTA |
| | CTACTTTGACTATTGGGGCCAGGGAACCCTGGTCA |
| | CCGTCTCCTCAGCGTCGACCAAGGGCCCATCGGTC |
| | TTCC |
| 0008-IGK | ATCCTTTTTCTAGTAGCAACTGCAACCGGTGTACA |
| | TTCTGACATCCAGATGACCCAGTCTCCATCCTCCC |
| | TGTCTGCATCTGTAGGAGACAGAGTCACCATCACT |
| | TGCCGGGCGAGTCTGGGCATTGGAAATTCTTTAGC |
| | CTGGTATCAGCAGAAACCAGGGAAAGCCCCTAAGG |
| | TCCTGCTCTATGCTGCATCCAGATTGGAAAGTGGG |
| | GTCCCATCCAGGTTCAGTGGCAGTGGATCTGGGAC |
| | GGATTACACTCTCACCATTAGCAGCCTGCAGCCTG |
| | AAGATTTTGCAACTTATTACTGTCAACAGTATTAC |
| | AGTACCCCTGAGGTCACTTTCGGCGGAGGGACCAA |
| | GGTGGAAATCAAACGTACGGTGGCTGCACCATCTG |
| | TCTT |
| 0011-IGG1 | ATCCTTTTTCTAGTAGCAACTGCAACCGGTGTACA |
| | TTCTGAGGTGCAGCTGGTGGAGTCTGGGGGAGGCG |
| | TGGTCCAGCCTGGGAGGTCCCTGAGACTCTCCTGT |
| | GCAGCCTCTGGATTCACCTTCAGTCGCTACGGCAT |
| | GCACTGGGTCCGCCAGGCTCCAGGCAAGGGGCTGG |
| | AGTGGGTGGCAGTTATATCATTTGATGGAAGGAAT |
| | AAATATTATGCAGACTCCGTGAAGGGCCGATTCAC |
| | CATCTCCAGAGACAATTCCAAAACCACGCTGTATT |
| | TGCAAATGAACAGCCTGAGAGCTGAGGACACGGCT |
| | GTGTATCACTGTGCGAAAGATGGGTTAGCAGTGTC |
| | GGACTACCTTGACTACTGGGGCCAGGGAACCCTGG |
| | TCACCGTCTCCTCAGCGTCGACCAAGGGCCCATCG |
| | GTCTTCC |
| 0012-IGK | ATCCTTTTTCTAGTAGCAACTGCAACCGGTGTACA |
| | TTCTGACATCCAGATGACCCAGTCTCCATCCTCCC |
| | TGTCTGCATCTGTAGGAGACAGAGTCACCATCACT |
| | TGCCGGGCAAGTCAGAGCATTACCAACTATTTAAA |
| | TTGGTATCAGCAGAAACCAGGGAAAGCCCCTGAGC |
| | TCCTGATCTATGCTGCATCCAGTTTGCAAAGTGGG |
| | GTCCCATCAAGGTTCAGTGGCAGTGGATCTGGGAC |
| | AGATTTCACTCTCACCATCAGCAGTCTGCAACCTG |
| | AAGATTTTGCAACTTACTACTGTCAACAGAGTTAC |
| | AGTACCCCTGGCACTTTTGGCCAGGGGACCAAGGT |
| | GGAAATCAAACGTACGGTGGCTGCACCATCTGTCT |
| | T |
| 0013-IGG1 | ATCCTTTTTCTAGTAGCAACTGCAACCGGTGTACA |
| | TTCTGAAGTGCAGCTGGTGGAGTCTGGGGGAGGCG |
| | TGGCCCAACCTGGGAGGTCCCTGAGACTCTCCTGT |
| | GCAGCCTCTGGATTCACCTTCAGTAACTATGCCAT |
| | GCACTGGGTCCGCCAGGCTCCAGGCAAGGGGCTGG |
| | AGTGGGTGGCAGTTATATCATTTGATGGAAGTGAT |
| | AAATACTATGTAGACTCCGTGAAGGGCCGATTCAC |
| | CATCTCCAGAGACAATTCCAAGAACACACTGTATC |
| | TGCAAATGAACAGCCTGAGAGCTGAGGACACGGCT |
| | GTGTATTACTGTGCGAAAAGCGGGGGGTTATATTG |
| | TAATGGTGGTAACTGCTACTACGGCTACTACTTTG |
| | ACTACTGGGGCCAGGGAACCCTGGTCACCGTCTCC |
| | TCAGCGTCGACCAAGGGCCCATCGGTCTTCC |
| 0014-IGK | ATCCTTTTTCTAGTAGCAACTGCAACCGGTGTACA |
| | TTCTGACATCCAGATGACCCAGTCTCCTTCCACCC |
| | TGTCTGCATCTGTAGGAGACAGAGTCACCATCACT |
| | TGCCGGGCCAGTCAGAGTATTAGTAGCTGGTTGGC |
| | CTGGTATCAGCAGAAACCAGGGAAAGCCCCTAAGC |
| | TCCTGATCTATGAGGCATCTAGTTTAGAAAGTGGG |
| | GTCCCATCAAGGTTCAGCGGCAGTGGATCTGGGAC |
| | AGAATTCACTCTCACCATCAGCAGCCTGCAGCCTG |
| | ATGATTTTGCAACTTATTACTGCCAACAGTATAAT |
| | ACTTATTCTCCGTACACTTTTGGCCAGGGGACCAA |
| | GGTGGAAATCAAACGTACGGTGGCTGCACCATCTG |
| | TCTT |
| 0015-IGG1 | ATCCTTTTTCTAGTAGCAACTGCAACCGGTGTACA |
| | TTCTGAAGTGCAGCTGGTGGAGTCTGGGGGAGGCG |
| | TGGTCCAGCCTGGGAGGTCCCTGAGACTCTCCTGT |
| | GCAGCCTCTGGATTCACCTTCAGTAGCTATGGCAT |
| | GCACTGGGTCCGCCAGGCTCCAGGCAAGGGGCTGG |
| | AGTGGGTGGCAGTTATATCATATGATGGAAGTAAT |
| | AAATACTATGCAGACTCCGTGAAGGGCCGATTCAC |
| | CATCTCCAGAGACAATTCCAAGAACACGCTGTATA |
| | TGCAAATGAACACCCTGAGAGCTGAGGACACGGCT |
| | GTGTATTACTGTGCGAAAGTTGTAGGAGCATATTG |
| | TGGTGGTGACTGCCTTACGGGATACTTTGACTACT |
| | GGGGCCAGGGAACCCTGGTCACCGTCTCCTCAGCG |
| | TCGACCAAGGGCCCATCGGTCTTCC |
| 0016-IGK | ATCCTTTTTCTAGTAGCAACTGCAACCGGTGTACA |
| | MCTGACATCCAGATGACCCAGTCTCCATCCTCCCT |
| | GTCTGCATCTGTAGGAGACAGAGTCACCATCACTT |
| | GCCAGGCGAGTCAGGACATTAGCAACTATTTAAAT |
| | TGGTATCAGCAGAAACCAGGGAAAGCCCCTAAGCT |
| | CCTGATCTACGATGCATCCAATTTGGAAACAGGGG |
| | TCCCATCAAGGTTCAGTGGAAGTGCATCTGGGACA |
| | GATTTTACTTTCACCATCAGCAGC |
| | CTGCAGCCTGAAGATATTGCAACATATTACTGTCA |
| | ACAGTATGATAATCTCCCTCTCACTTTCGGCGGAG |
| | GGACCAAGGTGGAAATCAAACGTACGGTGGCTGCA |
| | CCATCTGTCTT |
| 0017-IGG1 | ATCCTTTTTCTAGTAGCAACTGCAACCGGTGTACA |
| | TTCTGAAGTGCAGCTGGTGGAGTCTGGGGGAGGCG |
| | TGGTCCAGCCTGGGAGGTCCCTGAGACTCTCCTGT |
| | GCAGCCTCTGGATTCACCTTCAGTAGCCATGTTAT |
| | GCACTGGGTCCGCCAGACTCCAGGCAAGGGGCTGG |
| | AGTGGGTGGCGGTTATATCATATGATGGAAGCAGT |
| | AAATACTACGCAGACTCCGTGAAGGGCCGATTCAC |
| | CATCTCCAGAGACAATGCCAAGAACACGCTGTATC |
| | TGCAAATGAACAGCCTGAAAACTGAGGACACGGCT |
| | GTGTATTACTGTGCGAGAGAGCGAGTAAGCAGTGG |
| | CTGGTATCTTGATCCTTTTGATATCTGGGGCCAAG |
| | GGACAATGGTCACCGTCTCTTCAGCGTCGACCAAG |
| | GGCCCATCGGTCTTCC |
| 0018-IGK | ATCCTTTTTCTAGTAGCAACTGCAACCGGTGTACA |
| | TTCTGACATCCAGATGACCCAGTCTCCATCCTCCC |
| | TGTCTGCATCTGTAGGAGACAGAGTCACCATCACT |
| | TGTCGGGCAAGTCAGAGCATTAGCAACTATTTAAA |
| | TTGGTATCAGCAGAAACCAGGGAAAGCCCCTAAGC |
| | TCCTGATCTATGCTGCATCCAGTTTGCAAAGTGGG |
| | GTCCCATCAAGGTTCAGTGGCAGTGGATCTGGGAC |
| | AGATTTCACTCTCACCATCAGCAGTCTGCAACCTG |
| | AAGATTTTGCAACTTACTACTGTCAACAGAGTTAC |
| | ACTACCCTCTCGATCACCTTCGGCCAAGGGACACG |
| | ACTGGAGATTAAACGTACGGTGGCTGCACCATCTG |
| | TCTT |
| 0019-IGG1 | ATCCTTTTTCTAGTAGCAACTGCAACCGGTGTACA |
| | TTCTGAAGTGCAGCTGGTGGAGTCTGGGGGAGGCG |
| | TGGTCCAGCCTGGGAGGTCCCTGAGACTCTCCTGT |
| | GCAGCCTCTGGATTCACCTTCAGTAACTATGCTAT |
| | GCACTGGGTCCGCCAGGCTCCAGGCAAGGGGCTGG |
| | AGTGGGTGGCAGTTATATCATATGATGGAAGCAAT |
| | AAATACTATGTAGACTCCGTGAAGGGCCGATTCAC |
| | CATCTCCAGAGACAATTCCAAGAGCACGCTGTATC |
| | TGCAAATTAACAGCCTGAGAGCTGAGGACACGGCT |
| | GTCTATTACTGTGCGAGAGATCGCAAACCAAGTTA |
| | CGATTCTTGGAGTGGTTATACCCACTACCACTACG |
| | GTATGGACGTCTGGGGCCAAGGGACCACGGTCACC |
| | GTCTCCTCAGCGTCGACCAAGGGCCCATCGGTCTT |
| | CC |
| 0020-IGL2 | ATCCTTTTTCTAGTAGCAACTGCAACCGGTTCCTG |
| | GGCCCAGTCTGCCCTGACTCAGCCTGCCTCCGTGT |
| | CTGGGTCTCGTAGACAGTCGATCACCATCTCCTGC |
| | ACTGGAACCAGCAGTGATGTTGGGAGTTATAACCT |
| | TGTCTCCTGGTTCCAACATCACCCAGGCAAAGCCC |
| | CCAACCTCGTGATTTATGAGGACAATAAGCGGCCC |
| | TCAGGAGTTTCTAATCGCTTCTCTGGCTCCAAGTC |
| | TGGCCACACGGCCTCCCTGACAATCTCTGGGCTCC |
| | AGGCTGAGGACGAGGCTGATTATTACTGCTGCTCA |
| | TATGCAGGTAGTGGCACTTGGGTGTTCGGCGGAGG |
| | GACCAAGCTGACCGTCCTAAGTCAGCCCAAGGCTG |
| | CCCCCTCGGTCACTCTGTTCCCACCCTCGAGTGAG |
| | GAGCTTCAAGCCAACA |
| 0025-IGG1 | ATCCTTTTTCTAGTAGCAACTGCAACCGGTGTACA |
| | TTCCCAGGTGCAGCTGCAGGAGTCGGGCCCAGGAC |
| | TGGTGAAGCCTTCGGAGACCCTGTCCCTCACCTGC |
| | ACTGTCTCTGGTGGCTCCATCAGTAGTTACTACTG |
| | GAGCTGGATCCGGCAGCCCCCAGGGAAGGGACTGG |
| | AGTGGATTGGGTATATCTTTTACAGTGGGAGCACC |
| | AACTACAACCCCTCCCTCAGGAGTCGAGTCACCAT |
| | ATCAGTGGACACGCCCAAGAACCAGTTCTCCCTGA |
| | GGCTGAGGTCTGTGACCGCTGCGGACACGGCCGTG |
| | TATTACTGTGCGAGAGACTCTATGGATACAACTAC |
| | GTGGGCCCCTACGGCGTTTGACTACTGGGGCCAGG |
| | GAACCCTGGTCACCGTCTCCTCAGCGTCGACCAAG |
| | GGCCCATCGGTCTTCC |
| 0026-IGK | ATCCTTTTTCTAGTAGCAACTGCAACCGGTGTACA |
| | TTCTGACATCCAGATGACCCAGTCTCCATCCTCCC |
| | TGTCTGCATCTGTAGGAGACAGAGTCACCATCACT |
| | TGCCAGGCGAGTCAGGACATTAGCAACTATTTAAA |
| | TTGGTATCAGCAGAAACCAGGGAAAGCCCCTAAGC |
| | TCCTGATCTACGATGCATCCAATTTGGAAACAGGG |
| | GTCCCATCAAGGTTCAATGGAAGTGGATCTGGGAC |
| | AGATTTTACTTTCACCATCAGCAGCCTGCAGCCTG |
| | AAGATATTGCAACATATTACTGTCAACAGTATGAT |
| | AATCTCCCCCTCACTTTCGGCGGAGGGACCAAGGT |
| | GGAAATCAAACGTACGGTGGCTGCACCATCTGTCT |
| | T |
| 0031-IGG1 | ATCCTTTTTCTAGTAGCAACTGCAACCGGTGTACA |
| | TTCCGAGGTGCAGCTGGTGCAGTCTGGGGCTGAGG |
| | TGAAGAAGCCTGGGGCCTCAGTGAAGGTCTCCTGC |
| | AAGGCTTCTGGATACTTCTTCACCGGCTTCTACAT |
| | ACACTGGGTGCGACAGGCCCCTGGACAAGGGCTTG |
| | AGTGGATGGGATGGATCAGCCCTATCAGTGGTGGC |
| | GCAAACTCTGCACAGACGTTTCAGGACAGGGTCAC |
| | CATGACCAGGGACACGTCCATCACCACAGCCTACA |
| | TGGAGCTGAGCAGGCTGAGATCTGACGACACGGCC |
| | GTATACTACTGTGCGAG |
| | AGCCCCCTACTATGATAGCAGTGCTTCTCTTGACT |
| | ACTGGGGCCAGGGAACCCTGGTCACCGTCTCCTCA |
| | GCGTCGACCAAGGGCCCATCGGTCTTCC |
| 0032-IGK | ATCCTTTTTCTAGTAGCAACTGCAACCGGTGTACA |
| | TTCTGACATCCAGATGACCCAGTCTCCATCCTCCC |
| | TGTCTGCATCTGTAGGAGACAGAGTCACCATCACT |
| | TGCCAGGCGAGTCAGGACATTAGCAACTCTTTAAA |
| | TTGGTATCACCAGAAACCAGGGAAAGCCCCTAGGC |
| | TCCTGATCTACGATGCATCCAATTTGAAAACAGGG |
| | GTCCCATCAAGGTTCAGTGGAAGTGGATCTGGGAC |
| | AGATTTTACTTTCACCATCAGCAGCCTGCAGCCTG |
| | AAGATATTGGAACATTTTACTGTCAACAGTATGAT |
| | AATCTCCCTCCTGCCCTCACTTTCGGCCCTGGGAC |
| | CAAAGTGGATATCAAACGTACGGTGGCTGCACCAT |
| | CTGTCTT |
| 0039-IGG1 | ATCCTTTTTCTAGTAGCAACTGCAACCGGTGTACA |
| | TTCTGAGGTGCAGCTGGTGGAGTCTGGGGGAGGCT |
| | TGGTACAGCCTGGGGGGTCCCTGAGACTCTCCTGT |
| | GCAGCCTCTGGATTCACCTTCAGTAGGTACGACAT |
| | GCACTGGGTCCGCCAAGCTACAGGAAAAGGTCTGG |
| | AGTGGGTCTCAGCTATTGGTACTTCTGGTGACACA |
| | TACTATCCAGGCTCCGTGAGGGGCCGATTCACCAT |
| | CTCCAGAGAAAATGCCAAGAACTCCTTGTATCTTC |
| | AAATGAACAGCCTGAGAGCCGGGGACACGGCTGTG |
| | TATTACTGTGCAAGAGTCAACTATGATAGTGGTGG |
| | TTACGGAATACGGGAATACTGGTTCTTCGATCTCT |
| | GGGGCCGTGGCACCCTGGTCACCGTCTCCTCAGCG |
| | TCGACCAAGGGCCCATCGGTCTTCC |
| 0040-IGK | ATCCTTTTTCTAGTAGCAACTGCAACCGGTGTACAT |
| | TCTGACATCCAGATGACCCAGTCTCCATCCTCCCT |
| | GTCTGCATCTGTAGGAGACAGAGTCACCATCACTT |
| | GCCGGGCAAGTCAGAGCATTAGCAGCTTTTTAAAT |
| | TGGTATCAGCAGAAACCAGGGAAAGCCCCTAAACT |
| | CCTAATCTATGCTGCATCCAGTTTGCAGAGTGGGG |
| | TCCCATCAAGGTTCAGTGGCAGTGGATCTGGGACA |
| | GATTTCACTCTCACCATCAGCAGTCTCCAACCTGA |
| | AGATTTTGCAACTTACTTCTGTCAACAGAGTTACA |
| | GTACCCCTCCGTGGACGTTCGGCCAGGGGACCAAG |
| | GTGGAAATCAAACGTACGGTGGCTGCACCATCTGT |
| | CTT |
| 0043-IGG1 | ATCCTTTTTCTAGTAGCAACTGCAACCGGTGTACA |
| | TTCTGAGGTGCAGCTGTTGGAGTCGGGGGGAGGCT |
| | TGGTGCAGCCTGGGGGGTCCCTGAGACTCTCCTGC |
| | TCAGCTTCTGGATTCACCTTTGGCACCTATGCCAT |
| | GAGCTGGGTCCGCCAGGCTCCGGGGAAGGGGCTGG |
| | AGTGCGTCTCAACTATTGATGATATTTATGGTAGT |
| | GGTGGTAGGACCTTCTACGCAGGCTCCGTGCACGG |
| | CCGCTTCACCATTTCGAGAGACAATTCCAAGAACA |
| | CGCTGTATCTGCAGATGAACAGCCTGAGAGCCGAG |
| | GACACGGCCATATATTACTGTGCGAGAGATAAATA |
| | TCACTATGATAGTGGTGGTTATTATCGCCTGGCGG |
| | GACTTGACTACTGGGGCCAGGGAACCCTGGTCACC |
| | GTCTCCTCAGCGTCGACCAAGGGCCCATCGGTCTT |
| | CC |
| 0044-IGK | ATCCTTTTTCTAGTAGCAACTGCAACCGGTGTACA |
| | TTCAGACATCCAGTTGACCCAGTCTCCATCCTCCC |
| | TGTCTGCATCTGTAGGAGACAGAGTCACCATCACT |
| | TGCCGGGCCAGTCAGGGCATTAGCAGTTATTTAGC |
| | CTGGTATCAGCAAAAACCAGGGAAAGCCCCTAAGC |
| | TCCTGATCTTTGGTGCATCCACTTTGCAAAGTGGG |
| | GCCCCATCAAGGTTCCGCGGCAGTGGATCTGGGAC |
| | AGATTTCACTCTCGCCATCAGCAACCTGCAGCCTG |
| | AAGATTTTGCAACTTATTACTGTCAACAGACTGAT |
| | AGTTACCCTCGGACGTTCGGCCAAGGGACCAAGGT |
| | GGAAATCAAACGTACGGTGGCTGCACCATCTGTCT |
| | T |
| 0051-IGG1 | ATCCTTTTTCTAGTAGCAACTGCAACCGGTGTACA |
| | TTCTGAAGTGCAGCTGGTGGAGTCTGGGGGAGGCG |
| | TGGTCCAGCCTGGGAGGTCCCTGAGACTCTCCTGT |
| | GCAGCCTCTGGATTCACCTTCAGTAGCTATGGCAT |
| | GCACTGGGTCCGCCAGGCTCCAGGCAAGGGGCTGG |
| | AGTGGGTGGCTAGTATATCATATGATGGAAGTGAA |
| | TATTATGCAGAGTCCGTGAAGGGCCGATTCACCAT |
| | CTCCAGAGACAATTCCAAGAGCACGCTGCATCTGC |
| | AAATGAAAAGCCTGAGAGCTGAGGACACGGCTGTG |
| | TATTACTGTGCGAAAAATGGGGGGCCCTATTGTAG |
| | TGGTGGTGGCTGCTACGGATCGTACTTTGACTACT |
| | GGGGCCAGGGAACCCTGGTCACCGTCTCCTCAGCG |
| | TCGACCAAGGGCCCATCGGTCTTCC |
| 0052-IGK | ATCCTTTTTCTAGTAGCAACTGCAACCGGTGTACA |
| | TTCTGACATCCAGATGACCCAGTCTCCACCCTCCC |
| | TGTCTGCCTCTGTAGGAGACAGAGTCACCATCACT |
| | TGCCGGGCAAGTCAGAGCATTAGCAGCTATTTAAA |
| | TTGGTATCAGCAGAAACCAGGGAACGCCCCTAAGC |
| | TCCTGATCTTTGCTGCATCCAGTTTGGAAACTGGG |
| | GTCCCATCAAGGTTCAGTGGCAGTGGATCTGGGAC |
| | AGATTTCACTCTCACCATCAACAGTCTGCAACCTG |
| | AAGATTTTGCAACTTACTACTGCCAACAGAGTTCC |
| | AGTGCCCCCTTAACTTTCGGCCCTGGGACCAAAGT |
| | GGATATCAAACGTACGGTGGCTGCACCATCTGTCT |
| | T |
| 0053-IGK | ATCCTTTTTCTAGTAGCAACTGCAACCGGTGTACA |
| | TGGGGATATTGTGATGACCCAGACTCCACTCTCCC |
| | TGCCCGTCACCCTTGGACAGCCGGCCTCCATCTCC |
| | TGCAGGTCTAGTCAAAGCCTCGTATACAGTGATGG |
| | AAACACCTACTTGAATTGGTTTCAGCAGAGGCCAG |
| | GCCAATCTCCAAGGCGCCTAATTTATAAGGTTTCT |
| | AACCGGGACTCTGGGGTCCCAGACAGATTCAGCGG |
| | CAGTGGGTCAGGCACTCATTTCACACTGAAAATCA |
| | GCAGGGTGGAGGCTGAGGATGTTTGGCTTTATTAC |
| | TGCATGCAAGGTACACACTGGCTCTTCGGCGGAGG |
| | GACCAAGGTGGAAATCAAACGTACGGTGGCTGCAC |
| | CATCTGTCTT |
| 0086-IGG1 | ATCCTTTTTCTAGTAGCAACTGCAACCGGTGTACA |
| | TTCCGAGGTGCAGCTGGTGCAGTCTGGAGCAGAGG |
| | TGAAAAAGCCCGGGGAGTCTCTGAAGATCTCCTGT |
| | AAGGGTTCTGGATACAGCTTTATTAGCAACTGGAT |
| | CGGCTGGGTGCGCCAGATGCCCGGGAAAGGCCTGG |
| | AGTGGATGGGGAGCATCTATCCTGGTGACTCTGAC |
| | ACCAGATACAGTCCGTCCTTCCAAGGCCAGGTCAC |
| | CATCTCAGCCGACAAGTCCATCAGCACCGCCTACC |
| | TGCAGTGGAGCAGCCTGAAGGCCTCGGACACCGCC |
| | ATATATTACTGTGCGAGACTGGAGTCAGACTGGTA |
| | CTTCGATCTCTGGGGCCGTGGCACCCTGGTCACCG |
| | TCTCCTCAGCGTCGACCAAGGGCCCATCGGTCTTC |
| | C |
| 0087-IGL2 | ATCCTTTTTCTAGTAGCAACTGCAACCGGTTCTGT |
| | GACCTCCTATGAGCTGACWCAGGACCCTGCTGTGT |
| | CTGTGGCCTTGGGACAGACAGTCAGGATCACATGC |
| | CAAGGAGACAGCCTCAGAAGCCATTATGCAAGCTG |
| | GTACCAGCAGAAGCCAGGACAGGCCCCTGTAGTTG |
| | TCATCTATGGTAAAGACAACCGGCCCTCAGGGATC |
| | CCAGACCGATTCTCTGGCTCCAGCTCAGGAAATAC |
| | AGCTTCCTTGACCATCACTGGGGCTCAGGCGGAAG |
| | ATGAGGCTGACTACTACTGTAACTCCCGGGACAGC |
| | AGTGGAAACCATCCTTTCGGCGGAGGGACCAAGCT |
| | GACCGTCCTAGGTCAGCCCAAGGCTGCCCCCTCGG |
| | TCACTCTGTTCCCACCCTCGAGTGAGGAGCTTCAA |
| | GCCAACA |
| 0088-IGG1 | ATCCTTTTTCTAGTAGCAACTGCAACCGGTGTACA |
| | TTCCCAGGTGCAGCTGCAGGAGTCTGGGGGAGGCT |
| | TGGTCCAGCCTGGGGGGTCCCTGAGACTCTCCTGT |
| | GCAGCCTCTGGATTCACCGTCAGTAGCAACTACAT |
| | GAGCTGGGTCCGCCAGGCTCCAGGGAAGGGGCTGG |
| | AGTGGGTCTCACTTATTTATAGTGGTGGTAGCACA |
| | TACTACGCAGACTCCGTGAAGGGCAGATTCACCAT |
| | CTCCAGAGACTATTCCAAGAACACGCTGTATCTTC |
| | AAATGAACAGCCTGAGAGCCGAGGACACGGCTACG |
| | TATTATTGTGCGAGAGAACGTCCCCGCGGTGCGGG |
| | GGAGTACTGGGGCCAGGGAACCCTGGTCACCGTCT |
| | CCTCAGCGTCGACCAAGGGCCCATCGGTCTTCC |
| 0089-IGK | ATCCTTTTTCTAGTAGCAACTGCAACCGGTGTACA |
| | TTCTGACATCCAGATGACCCAGTCTCCATCGTCCC |
| | TGTCTGCATCTGTAGGAGACAGAGTCACCATCACT |
| | TGCCAGGCGAGTCAGGACATTAACATCTATTTAAA |
| | TTGGTATCAGCAGAAACCAGGAAAAGCCCCTAAGC |
| | TCCTGATCTACGATGCATCCAATTTGGAAACAGGG |
| | GTCCCATCAAGGTTCAGTGGAAGTGGATCTGGGAC |
| | AGATTTTACTTTCACCATCAGCAGCCTGCAGCCTG |
| | AAGATATTGCAACATATTACTGTCATCAGCATGAT |
| | AATCTCCCTCGGACTTTTGGCCAGGGGACCAAGGT |
| | GGAAATCAAACGTACGGTGGCTGCACCATCTGTCT |
| | T |
| 0092-IGG1 | ATCCTTTTTCTAGTAGCAACTGCAACCGGTGTACA |
| | TTCCGAGGTGCAGCTGGTGCAGTCTGGGGCTGAGG |
| | TGCAGAAGCCTGGGGCCTCAGTGAAGGTTTCCTGC |
| | AAGGCATCTGGACACACCTTCACCAGCTATTATAT |
| | ACACTGGGTGCGACAGGCCCCTGGACAAGGGCTTG |
| | AGTGGATGGGAATACTCAACACTAGTGGTGGTAGC |
| | ACAACCTACGCACAGAAGTTCCAGGGCAGAGTCAC |
| | CATGACCAGGGACACGTCCACGAGCACAGTCTACA |
| | TGGACCTGAGCAGCCTGAGATCTGAGGACACGGCC |
| | GTGTATTACTGTGCTTCGTCTTCGTGGGATGATGC |
| | TTTTGATATCTGGGGCCAAGGGACAATGGTCACCG |
| | TCTCTTCAGCGTCGACCAAGGGCCCATCGGTCTTC |
| | C |
| 0093-IGL2 | ATCCTTTTTCTAGTAGCAACTGCAACCGGTTCCAA |
| | TTCYCAGACTGTGGTGACYCAGGAGCCCTCACTGA |
| | CTGTGTCCCCAGGAGGGACAGTCACTCTCACCTGT |
| | GCTTCCAGCACTGGAGCAGTCACCAGTGGTTACTA |
| | TCCAAATTGGTTCCAGCAGAAACCTGGACAAGCAC |
| | CCAGGGCACTGATTTATAGTACAAGGAACAAACAT |
| | TCCTGGACCCCTGCCCGGTTCTCAGGCTCCCTCCT |
| | TGGGGGCAGAGCTGCCCTGACACTGTCAGGTGTGC |
| | AGCCTGAGGACGAGGCTGAGTATTACTGCCTGCTC |
| | TACTATGGTGGTCCTTGGGTGTTCGGCGGAGGGAC |
| | CAAGCTGACCGTCCTAGGTCAGCCCAAGGCTGCCC |
| | CCTCGGTCACTCTGTTCCCACCCTCGAGTGAGGAG |
| | CTTCAAGCCAACA |
| 0094-IGG1 | ATCCTTTTTCTAGTAGCAACTGCAACCGGTGTACA |
| | TTCCGAGGTGCAGCTGGTGCAGTCTGGGGCTGAGG |
| | TGAAGAAGCCTGGGGCCTCAGTGAA |
| | GGTTTCCTGCAAGGCATCTGGATACACCTTCATGA |
| | ACTATTATATGCACTGGGTGCGACAGGCCCCTGGA |
| | CAAGGGCTTGAGTGGATGGGAATGATCAACCCGAG |
| | TGGTGGTAGCGCAACCTACGCACAGAAGTTCCAGG |
| | GCAGAGTCACCATGACCAGGGACACGTCCACGAGC |
| | ACAGTTTACATGGAGCTGAGCAGCCTGAGATCTGA |
| | GGACACGGCCGTTTATTACTGTGCGAGAGAGGAGA |
| | GAGGTTGTAGTACTACCAGCTGCTATGATGATGCT |
| | TTTGATATTTGGGGCCAAGGGACAATGGTCACCGT |
| | CTCTTCAGCGTCGACCAAGGGCCCATCGGTCTTCC |
| 0095-IGK | ATCCTTTTTCTAGTAGCAACTGCAACCGGTGTACA |
| | TTCTGACATCCAGATGACCCAGTCTCCATCTGCCA |
| | TGTCTGCATCTGTAGGAGACAGAGTCACCATCACT |
| | TGTCGGGCGAGTCAGGGCATTAGCAATTATTTAGC |
| | CTGGTTTCAGCAGAAACCAGGGAAAGTCCCTAAGC |
| | GCCTGATCTATGCTGCATCCAGTTTGCAAAGTGGG |
| | GTCCCATCAAGGTTCAGCGGCAGTGGATCTGGGAC |
| | AGAATTCACTCTCACAATCAGCAGCCTGCAGCCTG |
| | AAGATTTTGCAACTTATTACTGTCTACAGCATAAT |
| | AGTTACCCTTGGACGTTCGGCCAAGGGACCAAGGT |
| | GGAAATCAAACGTACGGTGGCTGCACCATCTGTCT |
| | T |
| 0096-IGG1 | ATCCTTTTTCTAGTAGCAACTGCAACCGGTGTACA |
| | TTCCGAGGTGCAGCTGGTGCAGTCTGGGGCTGAGG |
| | TGAAGAAGCCTGGGGCCTCAGTGAAGGTTTCCTGC |
| | AAGGCATCTGGATACACCTTCATCACCTACTATAT |
| | ACACTGGATGCGACAGGCCCCTGGACAAGGGCTTG |
| | AGTGGATGGGACTAATCAACCCGAGTGGTGGTAGC |
| | ACAAACTTCGCACAGAACTTCCAGGGCAGAGTCAC |
| | CATGACCAGGGACACGTCCACGAGCACAGTCCACA |
| | TGGAGCTGACCAGCCTGAGATCTGAGGACACGGCC |
| | GTGTATTACTGTGCGAGAGGGGACTCCGGGTATAG |
| | CAGCAGCTGGTGTGATTACTGGGGCCAGGGAACCC |
| | TGGTCACCGTCTCCTCAGCGTCGACCAAGGGCCCA |
| | TCGGTCTTCC |
| 0097-IGL2 | ATCCTTTTTCTAGTAGCAACTGCAACCGGTTCTGT |
| | GACCTCCTATGAGCTGACWCAGGACCCTGCTGTGT |
| | CTGTGGCCTTGGGACAGACAGTCAGGATCACATGC |
| | CAAGGCGACAGCCTCAGAAGCTATTCTGCAAGCTG |
| | GTACCAGCAGAGGCCAGGACAGGCCCCTGTACTTG |
| | TCATCTATGCTAAAGACAACCGGCCCTCAGGGATC |
| | CCAGTCCGATTCTCTGGCTCCAGCTCAGGAACCAC |
| | AGCTTCCTTGACCATCACTGGGGCTCAGGCGGAAG |
| | ATGAGGCTGACTATTACTGTAGCTCCCGGGACAGC |
| | AGTGATACTGTGCTATTCGGCGGAGGGACCAAGTT |
| | GACCGTCCTAAGTCAGCCCAAGGCTGCCCCCTCGG |
| | TCACTCTGTTCCCACCCTCGAGTGAGGAGCTTCAA |
| | GCCAACA |
| 0100-IGG1 | ATCCTTTTTCTAGTAGCAACTGCAACCGGTGTACA |
| | TTCCGAGGTGCAGCTGGTGCAGGTGTCCAGTCCCA |
| | GGTCCAGCTGGTGACAGTCTGGGGCTGAGGTGAAG |
| | AAGCCTGGGTCCTCGGTGAAGGTCTCCTGCAAGGC |
| | TTCTGGAGGCACCTTCAGCTACTATGCTATCAACT |
| | GGGTGCGACAGGCCCCTGGACAAGGGCTTGAGTGC |
| | ATGGGAAGGATCATCCCTTTCCTTGGTATAGCAAA |
| | CTACACACAGAGATTCCAGGGCAGAGTCACGATTA |
| | CCGCGGACAAATCCACGAGCACAGCCTACATGGAG |
| | CTGCGCAGCCTGAGATCTGAGGACACGGCCGTATA |
| | TTTCTGTGCGAGAGAGGGGCCTTATTACTATGATA |
| | GTAGTGGTTACTCGAAATCCGACTCCGACGGTATG |
| | GACGTCTGGGGCCAAGGGACCACGGTCACCGTCTC |
| | CTCAGCGTCGACCAAGGGCCCATCGGTCTTCC |
| 0101-IGK | ATCCTTTTTCTAGTAGCAACTGCAACCGGTGTACA |
| | TTCAGACATCCAGTTGACCCAGTCTCCATCCTCCC |
| | TGTCTGCATCTGTAGGAGACAGAGTCACCATCACT |
| | TGCCGGGCCAGTCAGGGCATTAGCAGTTATTTAGC |
| | CTGGTATCAGCAAAAACCAGGGAAAGCCCCTAAGC |
| | TCCTGCTCTATGCTTCATCCACTTTGCCAAGTGGG |
| | GTCCCATCAAGGTTCAGCGGCAGTGGATCTGGGAC |
| | AGATTTCACTCTCACCATCAGCAGCCTGCAGCCTG |
| | AAGATTTTGCAACTTATTACTGTCAACAGCTTAAT |
| | AGTTACCCTCCCACTTTTGGCCAGGGGACCAAGGT |
| | GGAAATCAAACGTACGGTGGCTGCACCATCTGTCT |
| | T |
| 0104-IGG3 | ATCCTTTTTCTAGTAGCAACTGCAACCGGTGTACA |
| | TTCTGAGGTGCAGCTGGTGGAGTCTGGGGGAGGTG |
| | TGGTACGGCCTGGGGGGTCCCTCAGACTCTCCTGT |
| | GCAGCCTCTGGATTCACCTATGATACTTATGGGAT |
| | GAGTTGGGTCCGCCAAGCTCCAGGGAAGGGACTGG |
| | AGTGGGTCTCTGGTATTAATTGGAATGGTGGTAGG |
| | TCAGGTTATGCAGACTCTGTGAAGGGCCGATTCAT |
| | CATCTTCAGAGACAACGCCAAGAACTCCCTGTATC |
| | TGCAAATGAACAGTCTGAGAGTCGAGGACACGGCC |
| | TTATATTACTGTGCGAGAG |
| | CAAGCGTGGGATATTGTGCTAGTAGCAGGTGCTCC |
| | AACTGGTTCGACACCTGGGGCCAGGGAACCCTGGT |
| | CACCGTCTCCTCAGCGTCGACCAAGGGCCCATCGG |
| | TCTTCC |
| 0105-IGL2 | ATCCTTTTTCTAGTAGCAACTGCAACCGGTTCCTG |
| | GGCCCAGTCTGTGCTGACKCAGCCGCCCTCATTGT |
| | CTGCGGCCCCAGGACAGAAGGTCACCATCTCCTGC |
| | TCTGGAAGCAGCTCCAACATTGGGAATAATTATGT |
| | ATCTTGGTACCAACAACTCCCAGGAGCAGCCCCCA |
| | AACTCCTCATTTATGACAATAATAAGCGACCCTCA |
| | GGGATTTCTGACCGATTCTCTGGCTCCATGTCTGG |
| | CACGTCAGCCACCCTGGGCATCACCGGGCTCCAGA |
| | CTGGGGACGAGGGCGATTATTACTGCGGAACATGG |
| | GATAGCAGCCTGAGTCTTGTGGTGTTCGGCGGAGG |
| | GACCAAACTGACCGTCCTAGGTCAGCCCAAGGCTG |
| | CCCCCTCGGTCACTCTGTTCCCACCCTCGAGTGAG |
| | GAGCTTCAAGCCAACA |
| 0106-IGG1 | ATCCTTTTTCTAGTAGCAACTGCAACCGGTGTACA |
| | TTCTGAGGTGCAGCTGGTGGAGTCTGGGGGAGGCT |
| | TGGTCCAGCCTGGGGGGTCCCTGAGACTCTCCTGT |
| | GTAGCCTCTGGAATCACCGTCAGTGCCAATTACAT |
| | GAGCTGGGTCCGCCAGGCTCCAGGGAAGGGGCTGG |
| | AGTGGGTCTCAGTTATTTATAGCGGTGGTAGTACT |
| | TTCTACGCAGACTCCGTGAAGGGCAGGTTCACCAT |
| | CTCCAGAGACAATTCCAAGAACACTCTGTATCTTC |
| | AAATGAACAACCTGAGAGCCGACGACACGGCTGTG |
| | TATTCCTGCGCGAGAGATTTCAGGGGGGCAACTGC |
| | TTTTGATATCTGGGGCCAAGGGACAATGGTCACCG |
| | TCTCTTCAGCGTCGACCAAGGGCCCATCGGTCTTC |
| | C |
| 0107-IGL1 | ATCCTTTTTCTAGTAGCAACTGCAACCGGTTCCTG |
| | GGCCCAGTCTGCCCTGACTCAGCCTGCCTCCGTGT |
| | CTGGGTCTCCTGGACAGTCGATCACCATCTCCTGC |
| | ACTGCAACCAGCAGTGACGTTGATGATTATAACTA |
| | TGTCTCCTGGTACCAACAACACCCAGGCAAAGCCC |
| | CCAAACTCCTGATTTATGATGTCAATAATCGGCCC |
| | TCAGGGGTTTCCAATCGCTTCTCTGGCTCCAAGTC |
| | TGGCAACACGGCCTCCCTGACCATCTCTGGGCTCC |
| | AGGCTGAGGACGAGGCTGATTATTACTGCAGCTCA |
| | TATACAAGTAGCAGCACTGGAGTCTTCGGATCTGG |
| | GACCAAGGTCACCGTCCTAGGTCAGCCCAAGGCCA |
| | ACCCCACTGTCACTCTGTTCCCACCCTCGAGTGAG |
| | GAGCTTCAAGCCAACA |
| 0108-IGG1 | ATCCTTTTTCTAGTAGCAACTGCAACCGGTGTACA |
| | TTCTGAGGTGCAGCTGGTGGAGTCTGGGGGAGGCT |
| | TGGTCCAGCCTGGGGGGTCCCTGAGACTCTCCTGT |
| | GCAGCCTCTGGATTCACCGTCAGTAGCAACTACAT |
| | GAGCTGGGTCCGCCAGGCTCCAGGGAAGGGGCTGG |
| | AGTGGGTCTCAGTTATTTATAGCGGTGGTACCACA |
| | TACTACGCAGACTCCGTTAAGGGCAGATTCACCCT |
| | CTCCAGAGACAATTCCAAGAACACGCTGTATCTTC |
| | AAATGAACAGCCTGAGAGCCGAGGACACGGCTGTG |
| | TATTACTGTGCGAGGGGTTCCTATGATAGTAGTGG |
| | TTTGGTGATGAGTGGTGCTTTTGATATCTGGGGCC |
| | AAGGGACAATGGTCACCGTCTCTTCAGCGTCGACC |
| | AAGGGCCCATCGGTCTTCC |
| 0109-IGL2 | ATCCTTTTTCTAGTAGCAACTGCAACCGGTTCCTG |
| | GGCCCAGTCTGCCCTGACTCAGCCTCCCTCCGCGT |
| | CCGGGTCTCCTGGACAGTCAGTCACCATCTCCTGC |
| | ACTGGAACCAGCAGTGACGTTGGTGGTTATAACTA |
| | TGTCTCCTGGTACCAACAGCACCCAGGCAAAGCCC |
| | CCAAACTCATGATTTATGAGGTCAGTAAGCGGCCC |
| | TCAGGGGTCCCTGATCGCTTCTCTGGCTCCAAGTC |
| | TGGCAACACGGCCTCCCTGACCGTCTCTGGGCTCC |
| | AGGCTGAGGATGAGGCTGATTATTACTGCAGCTCA |
| | TATGCAGGCAGCAACAATTGGGTGTTCGGCGGAGG |
| | GACCAAGCTGACCGTCCTAGGTCAGCCCAAGGCTG |
| | CCCCCTCGGTCACTCTGTTCCCACCCTCGAGTGAG |
| | GAGCTTCAAGCCAACA |
| 0110-IGG1 | ATCCTTTTTCTAGTAGCAACTGCAACCGGTGTACA |
| | TTCTGAGGTGCAGCTGGTGGAGTCTGGGGGAGGCT |
| | TGGTCCAGCCTGGGGGGTCCCTGAAACTCTCCTGT |
| | GCAGCCTCTGGGTTCAGCTTCAGTGACTCTGCTAT |
| | GCACTGGGTCCGCCAGGCTTCCGGGAAAGGGCTGG |
| | AGTGGGTCGGCCGTATTAGAAGCAAACCTAACAAT |
| | TACGCGACAGCATATGCTGCGTCGGTGAAAGGCAG |
| | GTTCACCATCTCCAGAGATGATTCAAAGAACACGG |
| | CGTATCTGCAAATGAACAGCCTGAAAACCGAGGAC |
| | ACGGCCGTTTATTATTGTACTTCCTCTCCTCAACT |
| | GGAACTGTACGTGGACTACGGTATGGACGTCTGGG |
| | GCCAAGGGACCACGGTCACCGTCTCCTCAGCGTCG |
| | ACCAAGGGCCCATCGGTCTTCC |
| 0111-IGK | ATCCTTTTTCTAGTAGCAACTGCAACCGGTGTACA |
| | TTCTGACATCCAGATGACCCAGTCTCCTTCCACCC |
| | TGTCTGCATCTGTAGGAGACAGAGTCACCATCACT |
| | TGCCGGGCCAGTCAGAGTATTAGTAGCTGGTTGGC |
| | CTGGTATCAGCAGAAACCAGGGAAAGCCCCTAAGC |
| | TCCTGATCTATAAGGCATCTCGTTTACAAAGTGGG |
| | GTCCCATCAAGGTTCAGCGGCAGTGGATCTGGGAC |
| | AGAATTCACTCTCACCATCAGCAGCCTGCAGCCTG |
| | ATGATTTTGCAACTTATTACTGCCTACAGTATGAT |
| | ACTTACCCGTGGACCTTCGGCCAAGGGACCAAGGT |
| | GGAAATCAAACGTACGGTGGCTGCACCATCTGTCT |
| | T |
| 0114-IGG1 | ATCCTTTTTCTAGTAGCAACTGCAACCGGTGTACA |
| | TTCCCAGGTGCAGCTGCAGGAGTCGGGCCCAGGAC |
| | TGGTGAAGCCTTCACAGACCCTGTCCCTCACCTGC |
| | ACTGTCTCTGGTGGCTCCATCAATAGTGGTGGTTA |
| | CTACTGGAGCTGGATCCGCCAGCACCCAGGGAAGG |
| | GCCTGGAGTGGATTGGGTACATCCATTACAGTGGG |
| | AGCACCTACTACAGCCCGTCCCTCAAGAGTCGAAT |
| | TACCATATCAGTAGACACGTCTAAGGACCAGTTCT |
| | CCCTGAAGCTGAGCTCTGTGACTGCCGCGGACACG |
| | GCCGTATATTACTGTGCGAGAGAAAATGACTGGAG |
| | CTGGTTCGACCCCTGGGGCCAGGGAACCCTGGTCA |
| | CCGTCTCCTCAGCGTCGACCAAGGGCCCATCGGTC |
| | TTCC |
| 0115-IGK | ATCCTTTTTCTAGTAGCAACTGCAACCGGTGTACA |
| | TTCAGAAATTGTGTTGACACAGTCTCCAGCCACCC |
| | TGTCTTTGTCTCCAGGGGAAAGAGCCACCCTCTCC |
| | TGCAGGGCCAGTCAGAGTGTTAGGAGCTACTTAGC |
| | CTGGTACCAACAGAAACCTGGCCAGGCTCCCAGGC |
| | TCCTCATCTATGATGCATCCAACAGGGCCACTGGC |
| | ATCCCAGCCAGGTTCAGTGGCAGTGGGTCTGGGAC |
| | AGACTTCACTCTCACCATCAGCAGTCTAGAGCCTG |
| | AAGATTTTGCAGTTTATTACTGTCAGCAGCGTAGC |
| | AACTGGCCTAAGACGTTCGGCCAAGGGACCAAGGT |
| | GGAAATCAAACGTACGGTGGCTGCACCATCTGTCT |
| | T |
| 0116-IGG1 | ATCCTTTTTCTAGTAGCAACTGCAACCGGTGTACA |
| | TTCCCAGGTGCAGCTACAGCAGTGGGGCGCAGGAC |
| | TGTTGAAGCCTTCGGAAATCCTGTCCCGCACCTGC |
| | GCTGTCTTTGGTGGGTCCTTAAGCGGTTACTCTTG |
| | GAGCTGGATCCGCCAACCCCCAGGGAAGGGCCTGG |
| | AGTGGATTGGAGAAATCACTTATAGTGGAAACACC |
| | AGGTACAACCCGTCCCTCAAGAGTCGAGTCACCGT |
| | GTCAGTGGACACGTCCAAGAATCAGTTCTCCCTGA |
| | GGCTGAGTTCTGTGACCGCCGCGGACACGGCTGTA |
| | TATTTCTGTGCGAGAGTTATGAATGGAGTAGTACC |
| | ATCCCCTCTAGGGGGGCTGGGTCCATGGTACTCCT |
| | ACGACGCTATGGACGTCTGGGGCCAGGGGACCACG |
| | GTCACCGTCTCCTCAGCGTCGACCAAGGGCCCATC |
| | GGTCTTCC |
| 0117-IGL1 | ATCCTTTTTCTAGTAGCAACTGCAACCGGTTCTGT |
| | GACCTCCTATGAGCTGACWCAGGACCCTGCTGTGT |
| | CTGTGGCCTTGGGACAGACAGTCAGGATCACATGC |
| | CAAGGAGACAGTCTCAGAAAATATTATGCAAGTTG |
| | GTATCAACAGAAGCCAGGACAGGCCCCTGTACTTG |
| | TCATCTACGGTAAAAATAGCCGGCCCTCAGGGATC |
| | CCAGACCGATTCTCTGGCTCCACCTCAGGAGACAC |
| | AGCTTCCTTGGCCATCGCTGAGACTCAGGCGGAAG |
| | ACGAGGCTGAATACTACTGTCACTCCCGGGACAAC |
| | ACTGGTGACCATGTCTTCGGAACTGGGACCAAGGT |
| | CACCATTCTAGGTCAGCCCAAGGCCAACCCCACTG |
| | TCACTCTGTTCCCACCCTCGAGTGAGGAGCTTCAA |
| | GCCAACA |
| 0118-IGG1 | ATCCTTTTTCTAGTAGCAACTGCAACCGGTGTACA |
| | TTCCCAGGTGCAGCTGCAGGAGTCGGGCCCAGGAC |
| | TGCTGAAGCCTTCGGAGACCCTGTCCCTCAGCTGC |
| | ACTGTCTCTGGTGGCTCCATCAGTAGTTACTACTG |
| | GAGCTGGATCCGGCAGCCCCCAGGGAAGGGACTGG |
| | AGTGGATTGGGTATATCTATTACAGTGGGAGCACC |
| | AGTTACAACCCCTCCCTCAAGAGTCGAGTCGCCAT |
| | ATCAGTAGACACGTCCAAGAACCAGTTCTCCCTGA |
| | AGCTGAGCTCTGTGACCGCTGCGGACACGGCCGTG |
| | TATTACTGTGCGACCGATTACTATGATAGTAGTGG |
| | TTACTACTACGGTATGGACGTCTGGGGCCAAGGGA |
| | CCACGGTCACCGTCTCCTCAGCGTCGACCAAGGGC |
| | CCATCGGTCTTCC |
| 0119-IGK | ATCCTTTTTCTAGTAGCAACTGCAACCGGTGTACA |
| | TTCTGACATCCAGATGACCCAGTCTCCATCTTCCG |
| | TGTCTGCATCTGTTGGAGACAGAGTCACCATCACT |
| | TGTCGGGCGAGTCAGGATATTGGCAGCTGGTCAGC |
| | CTGGTATCAGCAGAAACCAGGGAAAGCCCCTAAGC |
| | TCCTGATCTATGCTGTATCCAATTTGCAAAGTGGG |
| | GTCCCATCAAGGTTCAGCGGCAGTGGATCTGGGAC |
| | AGATTTCACTCTCACCATCAGCGGCCTGCAGCCTG |
| | AAGATTTTGCAACTTACTATTGTCAACAGGCTAAC |
| | AGTTTCCGGACGTTCGGCCAAGGGACCAAGGTGGA |
| | AATCAAACGTACGGTGGCTGCACCATCTGTCTT |

### Exosomes Comprising ACE2, or Spike, or IgG

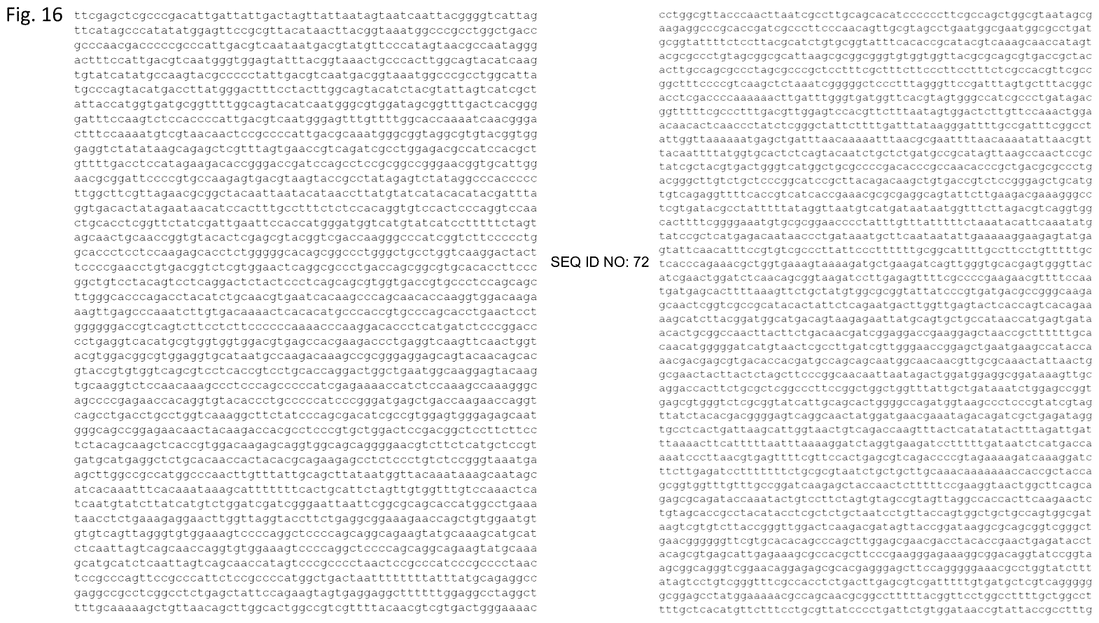

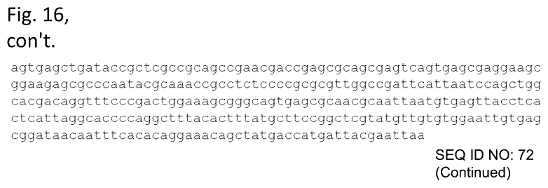

In some embodiments. a cell (e.g., a producer cell) is engineered to express, or overexpress ACE2. or spike, or IgG. or a fragment or variant thereof. By way of example, in some embodiments, the cell is transfected with an expression vector comprising an ACE2 nucleic acid sequence, a viral spike nucleic acid sequence, or an IgG sequence with paired H and L chains. In some embodiments, the engineered cell expressing the ACE2, the spike, and/or IgG (with TMD) also generates exosomes comprising the expressed ACE2, spike, and/or IgG. Exemplary, non-limiting vectors used to express ACE2 or spike include pcDNA3.1 and pDual-GFP. In some embodiments, the AbVec-hlg is may be used (see FIG. 16).

In some embodiments, the engineered cell comprises a mammalian cell. In some embodiments, the engineered cell comprises a HEK 293 cell. In some embodiments, the engineered cell comprises a HeLa cell. In some embodiments, the engineered cell comprises a dendritic cell. In some embodiments, the engineered cell comprises a B lymphocyte cell. In some embodiments, the engineered cell comprises a mesenchymal stem cell. In some embodiments, the engineered cell comprises a bone marrow-derived cell.

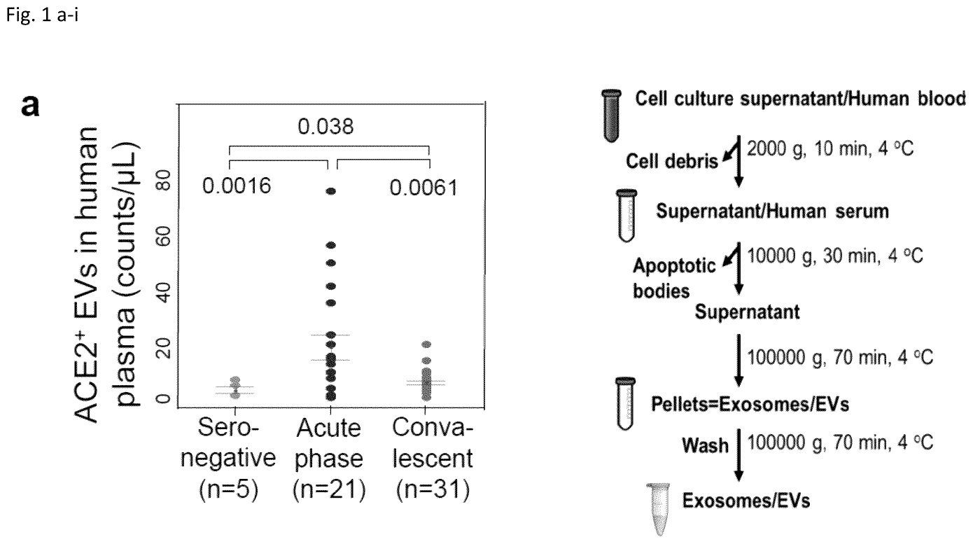

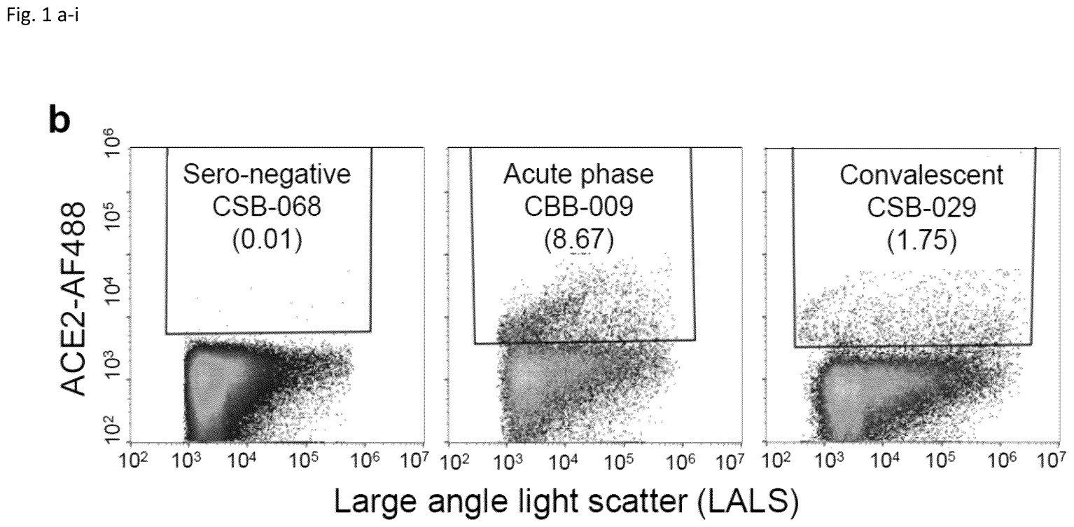

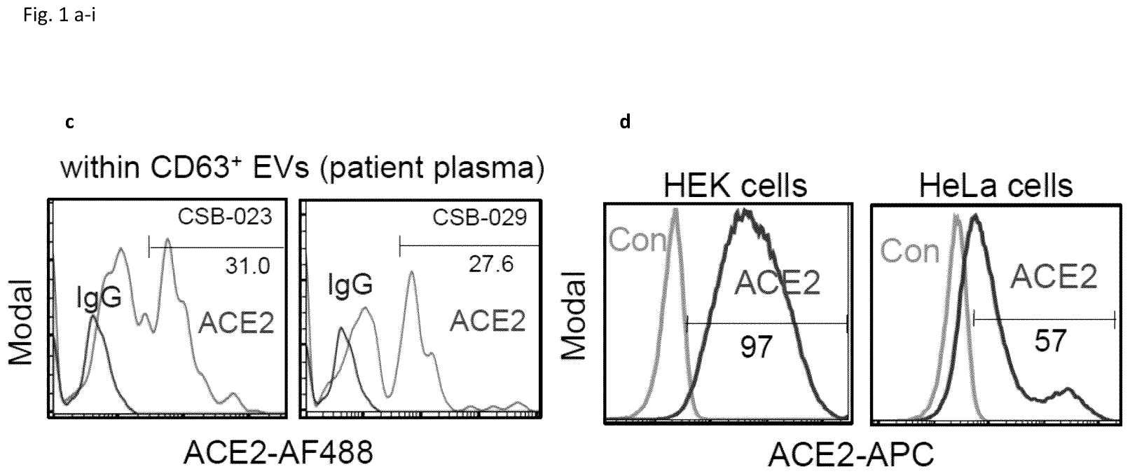

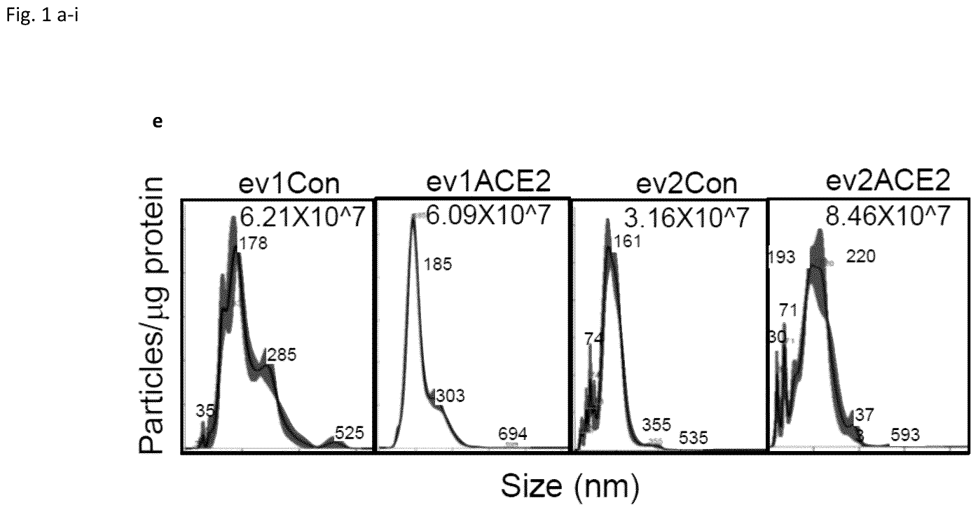

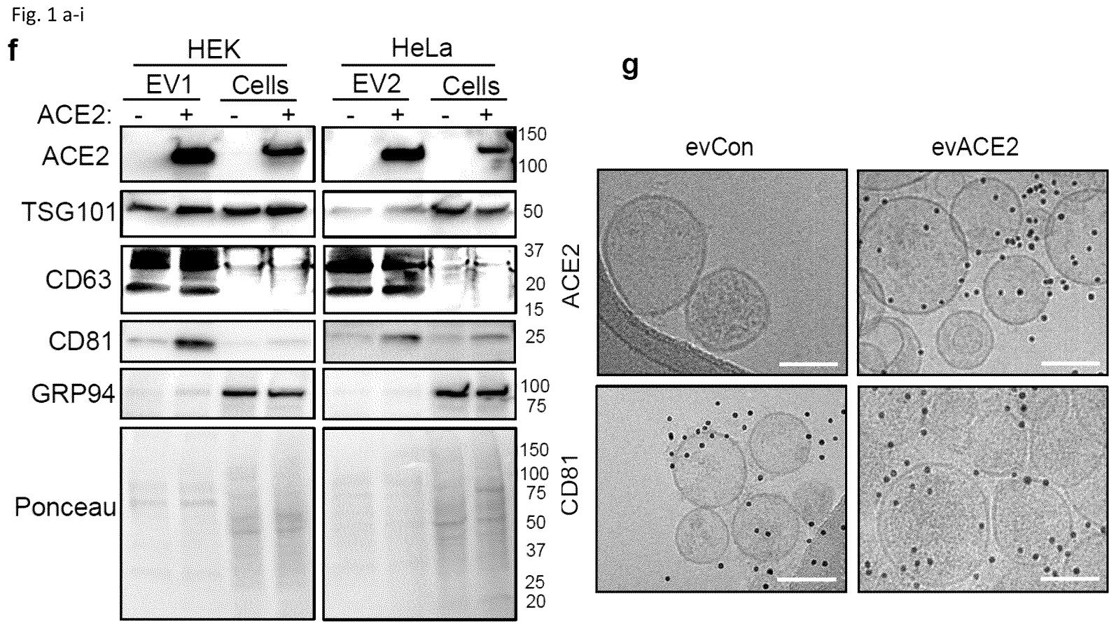

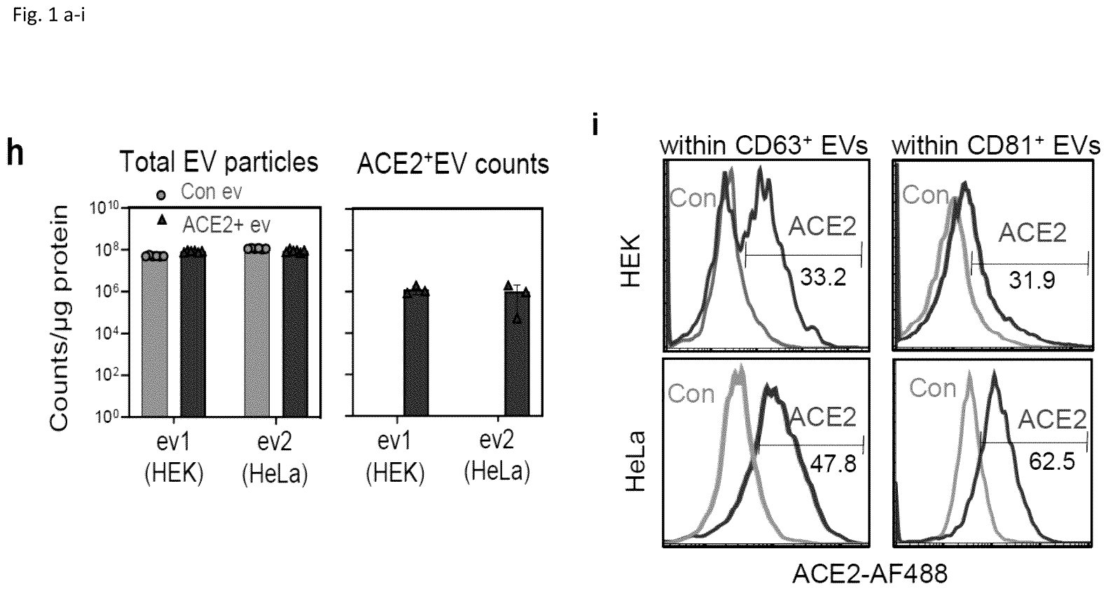

Isolation or purification of the ACE2 containing exosomes (extracellular vesicles) can be performed by methods well known in the art, for example, as shown in the steps of FIG. 1a, right panel.

Antibody or antigen/ligand capture techniques using columns and beads (e.g. magnetic beads) can be used to isolate exosomes presenting ACE2, spike, or Ig on the exterior surface, or to provide a quantitative value to the isolated exosomes with respect to ACE2.

In some embodiments, the exosome activity is quantitated against soluble human ACE2. In some embodiments, ACE2-expressing exosomes, spike-expressing exosomes, and Ig-expressing exosomes are quantitated on micro flow cytometry (vesiclometry). In some embodiments, 0.5 μg of an exosome composition comprising ACE2 is equivalent to about 140 ng of soluble human ACE2. In some embodiments, each exosome presents about between about 100 and 2000 ACE2 molecules, between about 500 and 1500 ACE2 molecules or about 1000 ACE2 molecules.

### SARS-COV-2 Infection

Since December 2019, the outbreak of a novel coronavirus, severe acute respiratory syndrome coronavirus 2 (SARS-COV-2), infection (coronavirus disease 2019 [COVID-19]) that started in Wuhan, Hubei Province, People's Republic of China, is now a global pandemic. Although patients initially present with fever with or without respiratory symptoms, various degrees of pulmonary abnormalities develop later in all patients, and these are typically observable via chest computed tomography (CT) imaging. Most patients only have a common, mild form of illness, but approximately 15% to 20% fall in the severe group, meaning they require assisted oxygenation as part of treatment. The severe group has a high mortality rate and is associated with older age, underlying diseases such as diabetes, and medical procedures (such as patients who were infected in a hospital setting while undergoing an operation for other indications). Although there have been several studies describing clinical features and characteristic radiographic findings (mainly chest CT scans), limited pathologic studies have been conducted on the basis of autopsies or biopsies. Some of the reasons for the lack of autopsies and biopsies include suddenness of the outbreak, vast patient volume in hospitals, shortage of health care personnel, and high rate of transmission, which makes invasive diagnostic procedures less of a clinical priority. Even so, information regarding the lung pathology and histology of subjects who have died of COVID-19 complication has been growing.

Pathological findings in the lungs of early stage COVID-19 patients include edema, proteinaccous exudate, focal reactive hyperplasia of pneumocytes with patchy inflammatory cellular infiltration, and multinucleated giant cells; typically, hyaline membranes were not prominent. Biopsies from the lungs of later stage COVID-19 subjects exhibit one or more of desquamation of pneumocytes and hyaline membrane formation indicative of acute respiratory distress syndrome (ARDS); pulmonary edema with hyaline membrane formation, suggestive of carly-phase ARDS; interstitial mononuclear inflammatory infiltrates dominated by lymphocytes; multinucleate syncytial cells with atypical enlarged pneumocytes characterized by large nuclei, amphophilic granular cytoplasm, prominent nucleoli in the intra-alveolar spaces, showing viral cytopathic-like changes. Histological characteristics may include bilateral diffuse alveolar damage with cellular fibromyxoid exudates. (See e.g., Tian, et al, Journal of Thoracic Oncology, vol. 15 no. 5:700-704, 28 Feb. 2020; Xu, et al., The Lancet, at world wide web dot the lancet dot com front slash respiratory, vol. 8; 420-422, April 2020, herein incorporated by reference).

Methods of identifying a viral infection, such as a SARS-COV-2 infection are well known in the art and include molecular testing (e.g., to detect viral genetic material), antigen testing (e.g., to detect viral proteins), and antibody testing (e.g., testing the subject for antibodies directed to the virus).

### Pharmaceutical Therapeutic Compositions, Formulations, and Modes of Administration

Disclosed herein are therapeutic compositions useful in the treatment or prevention of symptoms associated with infection by SARS-COV-2, and other coronaviruses such as HCoV-NL63 and SARS-COV. For exemplary purposes only, the discussion below relates to SARS-COV-2, but the methods and compositions herein are not intended to be so limited and would apply to any virus that targets the ACE2 protein to effect cellular entry or further viral infection.

The pandemic of COVID-19 is caused by infections of human corona virus SARS-CoV-2 through its spike protein interactions with human receptor ACE2 on host cells. Disclosed herein are biological product, ACE2+ exosomes (extracellular vesicles), from two human cell lines (HEK-293 and Hela) with overexpressing cDNA of ACE2. The ACE2+ exosomes can bind to viral proteins, such as SARS-COV-2, and block SARS-COV-2 spike protein (RBD) attachment to human cells, and therefore serve as a promising neutralizing therapeutics in treating COVID-19.

As described herein, human ACE2 cDNA was overexpressed in HEK-293 cells and Hela cells that produce and secret ACE2+ exosomes. ACE2+ exosomes were purified from the culture supernatant of these cells and their biochemical properties were characterized, including sizes and protein expression profiles, and their neutralization capacity in SARS-COV-2 viral protein binding to human ACE2+ cells was measured. The examples herein demonstrate that ACE2+ exosomes in a size of about 100 nm can compete with cellular ACE2 on plasma membrane to inhibit viral protein attachment to human host cells, such as ACE2+ HEK-293, which are susceptible to corona viral infections. Furthermore, ACE2+ exosomes inhibit SARS-COV-2 spike+ pseudoviral infections to ACE2+ Hela cells.

In addition to ACE2+ exosomes, based on convalescent B cell VDJ sequencing, SARS-CoV-2 specific IgG cDNA expression vectors with or without its transmembrane domain were created. The SARS-COV-2 neutralizing IgG and IgG+ exosomes produced by HEK-293 cells will be administered for therapeutic treatment and/or employed for diagnostic applications.

Accordingly, pharmaceutical and therapeutic compositions disclosed herein comprise exosomes comprising ACE2, exosomes comprising SARS-COV-2 specific IgG (H+L), and soluble IgG (H+L) that binds to or neutralizes SARS-COV-2. Such compositions may be formulated for administration by any suitable route of delivery, such as oral, parenteral, rectal, nasal, topical, or ocular routes, or by inhalation. By way of example but not by way of limitation, the therapeutic exosome compositions disclosed herein can be formulated for administered by inhalation.

A “subject in need of treatment” may include a subject diagnosed with, suspected of having, or at risk of infection by SARS-COV-2 or other any other virus such as HCoV-NL63 and SARS-COV that utilize ACE2 as a cellular entry point or to further viral infection.

The term “combination therapy” is used in its broadest sense and means that a subject is administered at least two agents. More particularly, the term “in combination” with respect to therapy administration refers to the concomitant administration of two (or more) active agents for the treatment of a disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form (e.g., exosomes presenting ACE2 are loaded with remdesivir). In another embodiment, the active agents are administered in separate dosage forms.

By way of example but not by way of limitation, additional active agents include remdesivir (a RNA analog inhibiting SARS-COV-2 RNA synthesis). Disclosed herein are optimized exosome RNA loading protocols using electroporation.

### Exemplary Exosome Loading Protocol

To prepare exosomes for RNA oligo loading, 3 μg of fresh or thawed exosomes are placed on ice and mixed with 100 nM oligonucleotide final concentration in 400 uL of electroporation buffer. Electroporation buffer is made according to Kooikmans et al., 2013. Transfer the mixture to a 0.4 mL cuvette for electroporation and electroporate at 400V and 125 uF one pulse. Immediately place the samples on ice for at least 30 mins. Transfer the electroporated samples to an appropriate ultracentrifuge tube and wash once 100,000×g in PBS. Resuspend the exosomes in PBS and proceed with analysis.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. In some embodiments, such combination therapies utilize lower dosages of the conventional therapeutics, thus avoiding possible toxicity and adverse side effects incurred when those agents are used as monotherapies.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic.

The agents, whether administered alone or in combination, may be administered multiple times, and if administered as a combination, may be administered simultaneously or not, and on the same schedule or not. By way of example, a therapeutic composition may be administered multiple times per day, once per day, multiple times per week, once per week, multiple times per month, once per month, or as often as a doctor prescribes.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually. By way of example, but not by way of limitation, in some embodiments, a therapeutic composition comprises an exosome presenting ACE2

In therapeutic applications, the compounds of the disclosure can be formulated for a variety of modes of administration, including systemic and topical or localized administration. Such modes of administration include but are not limited to formulations for oral, parenteral, iv, inhalation, intranasal, and direct injection or administration to an affected organ or tissue. Techniques and formulations generally may be found in Remington: The Science and Practice of Pharmacy (20^{th} ed.) Lippincott, Williams and Wilkins (2000).

Use of pharmaceutically acceptable inert carriers or vehicles, such as oily or aqueous vehicles or carries, to formulate the compounds herein disclosed for the practice of the disclosure into dosages suitable for administration is within the scope of the disclosure. With proper choice of carrier and suitable manufacturing practice, the compositions of the present disclosure, in particular, those formulated as solutions or emulsions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, dragees, liquids, gels, syrups, slurries, suspensions, emulsions, and the like, for oral ingestion. For nasal or inhalation delivery, the compositions of the disclosure also may be formulated by methods known to those of skill in the art, and may include, for example, but not limited to, cartridges, emulsions, sprays, inhalers, vapors; solubilizing, diluting, or dispersing substances, such as saline, preservatives, such as benzyl alcohol; absorption promoters; and fluorocarbons may be included.

In some embodiments, the composition is administered via one or more of the following routes: orally (e.g., as a capsule, tablet, liquid, or gel), parenterally (e.g., subcutancously, intramuscularly, or intravenously), topically (e.g., as a cream or lotion, via iontophoresis, or transdermally, e.g., via a patch), and via inhalation.

In some embodiments, the therapeutic agent is directly administered as a pressurized aerosol or nebulized formulation to the patient's lungs via inhalation. Such formulations may contain any of a variety of known aerosol propellants useful for endopulmonary and/or intranasal inhalation administration. In addition, water may be present, with or without any of a variety of cosolvents, surfactants, stabilizers (e.g., antioxidants, chelating agents, inert gases and buffers). For compositions to be administered from multiple dose containers, antimicrobial agents are typically added. Such compositions are also generally filtered and sterilized, and may be lyophilized to provide enhanced stability and to improve solubility.

In some embodiments, compositions of the present disclosure are administered to a subject in need thereof as a single dose, one time. In some embodiments, compositions of the present invention are administered to a subject for multiple times, and in some embodiments, may be administered as part of a regimen (e.g., multiple administrations over a period of time). For example, a regimen may comprise one or more doses administered to a subject in need thereof daily, weekly, bi-weekly, or monthly. In some embodiments, a regimen comprises multiple administrations over the course of one week; two weeks; three weeks; four weeks; five weeks; or six weeks. In some embodiments, a regimen comprises multiple administrations over the course of one month; two months; three months; four months; or five months. In some embodiments, a regimen comprises multiple administrations over the course of a year. In some embodiments, a regimen comprises administration once per week; administration once every other day; or administration once per day. In some embodiments, the regimen provides for an initial dose, followed by one dose per week for about 3 to about 12 weeks, or for about 4 weeks, for about 5 weeks, about 6 weeks, about 7 weeks, about 8 weeks, about 9 weeks, about 10 weeks, or about 11 weeks. In some embodiments, a regimen includes one or more initial doses, followed by weekly, bi-weekly, or monthly doses. For example, a regimen may include one or more initial doses over the course of 1 day, 2 days, 3 days, 4 days or 5 days, followed by one dose every day, every other day, or once per week for up to 1 to about 12 weeks, or one dose per month for up to about 4 months. In some embodiments, a regimen includes one dose every other day for about 1 to about 4 weeks, or one dose every day for about 3 days to about 1 week. In some embodiments, a dosage regimen comprises an initial dose, followed by one dose per week for 3 weeks, 4 weeks, 5 weeks or 6 weeks.

Dosage and administration of the exosome compositions alone or in combination with additional therapeutic agents can be determined by a skilled artisan using methods that are standard in the art, based on patient age, weight, sex, race, overall health, stage of the disease. Determination of the effective amounts is well within the capability of those skilled in the art, especially in light of the detailed disclosure provided herein.

Generally, the compounds according to the disclosure are effective over a wide dosage range. For example, in the treatment of adult humans, dosages from 0.1 to 1000 mg/kg, from 0.5 to 200 mg/kg, from 1 to 50 mg per day/kg, and from 5 to 40 mg/kg per day are examples of dosages that may be used. A non-limiting dosage is 5 to 30 mg/kg per day. In some exemplary embodiments, the dosage is 0.1-100 mg/kg, a non-limiting dosage is 1-10 mg/kg per day. By way of example, the dosage for soluble IgG, IgG+exosomes, or ACE2+ exosomes is 0.1-100 mg/kg, a non-limiting dosage is 1-5 mg/kg per day. In some exemplary embodiments, the dosage for soluble IgG, IgG+ exosomes, or ACE2+ exosomes is 0.1-100 mg/kg, a non-limiting dosage is 1-10 mg/kg per day. In some exemplary embodiments, the dosage for combination of IgG+ or ACE2+ exosomes loaded with remdesivir is 0.1-50 mg/kg and 0.1-100 mg/kg per day, respectively, a non-limiting dosage is 1-10 mg/kg per day for ACE2 and 0.5-10 mg/kg per day for remdesivir. In some exemplary embodiments, the dosage for combination of ACE2+ exosomes loaded with remdesivir is 0.1-50 mg/kg and 0.1-100 mg/kg per day, respectively, a non-limiting dosage is 1-10 mg/kg per day for ACE2 and 1-50 mg/kg for remdesivir. The routine of administration to treat symptoms of SARS-COV-2 infection may be oral, parenteral, iv, inhalation, intranasal.

The exact dosage will depend upon the route of administration, the form in which the compound is administered, the subject to be treated, the body weight of the subject to be treated, the viral load, disease severities and the preference and experience of the attending physician.

In some embodiments, improvement in a patient's condition, as compared to an untreated control subject, is noted within about a day, a week, two weeks, or within about one month after the first treatment is administered. By way of example, improvements in a patient's condition may include, but is not limited to an improvement in clinical symptoms, test results (e.g., viral titer, patient antibodies), histopathological findings, quality of life, and longevity.

### Discussion

Recent studies demonstrated that soluble human ACE2 inhibits SARS-COV-2 infections in engineered human tissues. As shown in Example 1, human ACE2 cDNA was overexpressed in in HEK-293 cells and Hela cells that produce and secret ACE2+ exosomes. We purified ACE2+ exosomes from the culture supernatant of these cells and measured their neutralization capacity in SARS-COV-2 viral protein binding to human ACE2+ cells. Example 1 demonstrated that ACE2+ exosomes can compete with cellular ACE2 on plasma membrane to inhibit viral protein attachment to human host cells, such as ACE2+ HEK-293 susceptible to corona viral infections.

We have created the biological product, named “decoy exosomes” that show neutralizing effects to block viral infection. The decoy exosomes (particles in size, in some embodiments, of ˜100 nanometer) can not only present proteins to inhibit virus entry to human cells, but also load RNA cargo therapeutics such as remdesivir to deliver to human cells. As shown in Example 1, exosomal ACE2 competes with cellular ACE2 binding to the RBD of SARS-COV-2 Spike protein. In comparison to soluble ACE2, exosomal ACE2 has better neutralization capacity and efficacy in inhibiting viral spike RBD binding to human host cells as well as spike+ pseudoviral infections to human cells.

The advantages of the present technology include, but are not limited to transmembrane protein conformation with the full length ACE2 protein, longer plasma half-life, targeted delivery to the lungs for COVID-19 treatment, and loading capacity with anti-viral RNA-analogs like remdesivir that have shown the clinical benefits of shortening recovery time of COVID-19.

In addition, in comparison to anti-SARS-COV-2 neutralizing antibodies, exosomal ACE2 as a decoy therapy can target all corona viruses and their broad strains including mutants that use human ACE2 as an entry receptor.

### EXAMPLES

The following Examples are illustrative and should not be interpreted to limit the scope of the claimed subject matter.

### Example 1. ACE2− Expressing Extracellular Vesicles as an Innate Antiviral Response and a Decoy Therapy to Block Broad Strains of SARS-COV-2

Abstract The severe acute respiratory syndrome coronavirus 2 (SARS-COV-2) has caused the pandemic of the coronavirus induced disease 2019 (COVID-19) with 216 million cases and 4.5 million deaths as of September 2021. That has been exacerbated by mutated variants such as α, β, and δ with high infection rates and increased breakthroughs, resulting in vaccine inefficiencies and resistance to therapeutic monoclonal antibodies. It remains urgent to identify novel therapeutics against broad strains of SARS-COV-2 and future emerging corona viruses to protect the immune-compromised, unvaccinated, and even vaccinated from breakthrough infections. Wild-type SARS-COV-2 and its variants infect human cells and other hosts' cells via the entry receptor angiotensin-converting enzyme 2 (ACE2), triggering innate and adaptive immune responses. Herein, we report an increase in circulating extracellular vesicles (EVs) that express ACE2 (evACE2) in plasma of both acute and convalescent COVID-19 patients as part of the innate antiviral response associated with severe pathogenesis. Furthermore, evACE2 isolated from both human plasma and engineered EV-producing cell lines neutralizes SARS-COV-2 infection by competing with cellular ACE2. Notably, compared to vesicle-free recombinant human ACE2 (rhACE2), evACE2 shows a 135-fold higher potency in blocking the binding of the viral spike (S) protein RBD to ACE2⁺ cells, and a 60- to 80-fold higher efficacy in preventing infections by both pseudotyped and authentic SARS-COV-2 in permissive cells. Consistently, evACE2 protects the hACE2 transgenic mice from SARS-COV-2-induced lung injury and mortality. More importantly, evACE2 inhibits the infection of SARS-COV-2 variants (α, β, and δ) with equal or higher potency than for the wildtype strain, supporting a broad-spectrum antiviral mechanism of evACE2 for therapeutic development to block the infection of existing and future corona viruses that use the ACE2 receptor.

Introduction Despite the tremendous success of COVID-19 vaccine development, the pandemic caused by SARS-COV-2 has been challenging due to fast evolving mutant strains¹⁻⁵ and slow vaccination globally. Shortly after the outbreak, over 1,000 SARS-COV-2 variants were detected⁶ and several mutant strains (α, β, and δ) dominated with higher infection rates and/or fatality than the wild-type (WT) strain^{4,5,7,8.} Fully vaccinated populations only reached lower than 30% worldwide and about half in the US as of September 2021. Moreover, the risk of future emerging corona viruses to infect human always exist. To better protect vulnerable people, both unvaccinated and vaccinated, it is urgent to develop novel therapeutics that can broadly target distinct strains of evolving SARS-COV-2 and future corona viruses.

Similar to other corona viruses such as SARS-COV, which caused an outbreak in 2003⁹, the WT and mutant strains of SARS-COV-2 infect host cells such as human pneumocytes via the entry receptor angiotensin-converting enzyme 2 (ACE2)^{1,10-12}. The mutations-caused alterations in the viral proteins such as the attachment protein—spike glycoprotein (S), in particular the external receptor-binding domain (RBD) in the variants (α, β, and δ), render a greater binding affinity than the WT in binding to ACE2^{1,4,5,10-12}. Approaches to block or impede the viral interaction with the entry receptor ACE2 on the host cell, including S-specific neutralization antibodies (Abs)¹³⁻²⁵ and rhACE2²⁶⁻³⁰, inhibit infectivity and prevent COVID-19. Although many high-affinity monoclonal antibodies were identified from convalescent patients and engineered as therapeutics to treat mild diseases of COVID-19¹³⁻²⁵, many did not show favorable efficacy for hospitalized patients^{31,32} and some of those with emergency use authorization (EUA) had lost efficacy against new variants, such as δ³³. Monoclonal antibodies targeting specific epitopes of SARS-COV-2 antigens appear to have limited capacity to broadly neutralize current and future mutant strains⁴⁻⁶. Nonetheless, since the plasma or sera of convalescent COVID-19 patients have reportedly been used to treat active infection of SARS-COV-2 or severe diseases^{34,35}, we aimed to identify new anti-viral components from the human plasma that may inform on potential new therapeutics.

Extracellular vesicles (EVs) are one of the essential components of liquid biopsy such as blood, including large microvesicles (200-1,000 μm), small exosomes (50-200 μm), and newly identified exomeres (<50 μm)^{36,37}. Exosomes are amongst the best characterized small EVs that likely participate in a variety of physiological and pathobiological functions³⁸⁻⁴² as well as serve as novel biomarkers and next-generation biologic therapeutics^{43,44}. They present many proteins on the surface reminiscent of their cellular counterpart, such as immune regulators of myeloid and lymphoid cells to affect antiviral immune response^{42,45,46}. Exosomes derived from both plants and human specimens have been used in clinical trials to treat inflammatory diseases and cancers⁴⁷⁻⁴⁹. In line with widely adopted nomenclature in the EV field³⁷ and the possibility that heterogenous vesicle populations may be isolated, we collectively refer to the enriched exosomes therein as ‘EVs’.

Here we detected a significant increase in circulating ACE2+ EVs in the plasma of COVID-19 patients, in particular during the acute phase. Importantly, ACE2⁺ EVs (evACE2) isolated from engineered cell lines inhibit SARS-COV-2 infection by blocking the viral spike protein binding with its cellular receptor ACE2 in host cells. Our observations demonstrate that evACE2 is a previously unknown innate antiviral mechanism to prevent SARS-COV-2 infection, thus providing a rationale for the use of evACE2 to combat COVID-19.

### Results

Circulating evACE2 dramatically increased in the peripheral blood of COVID-19 patients

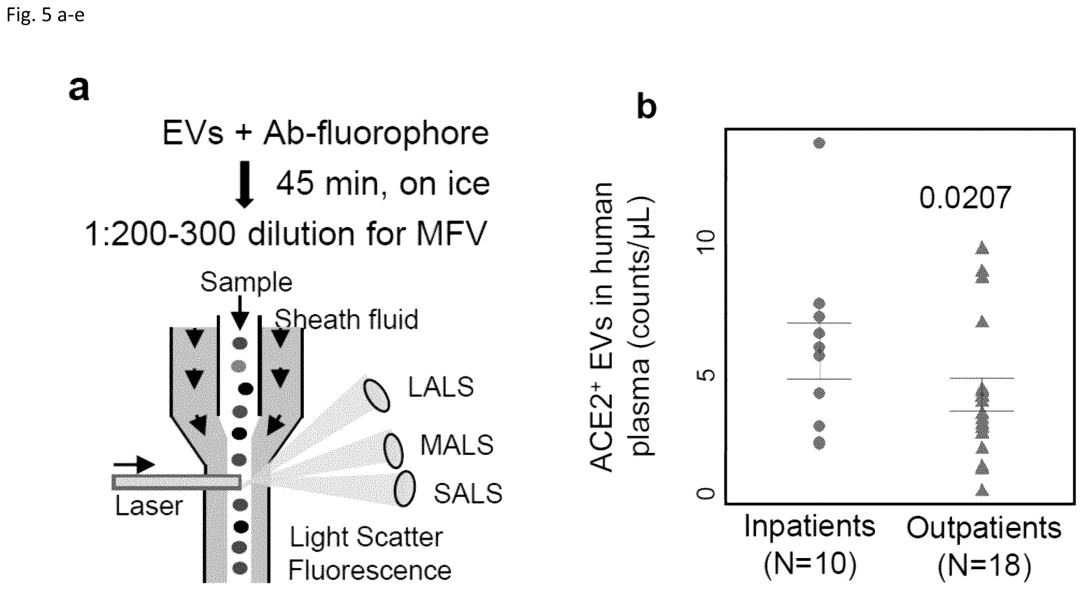

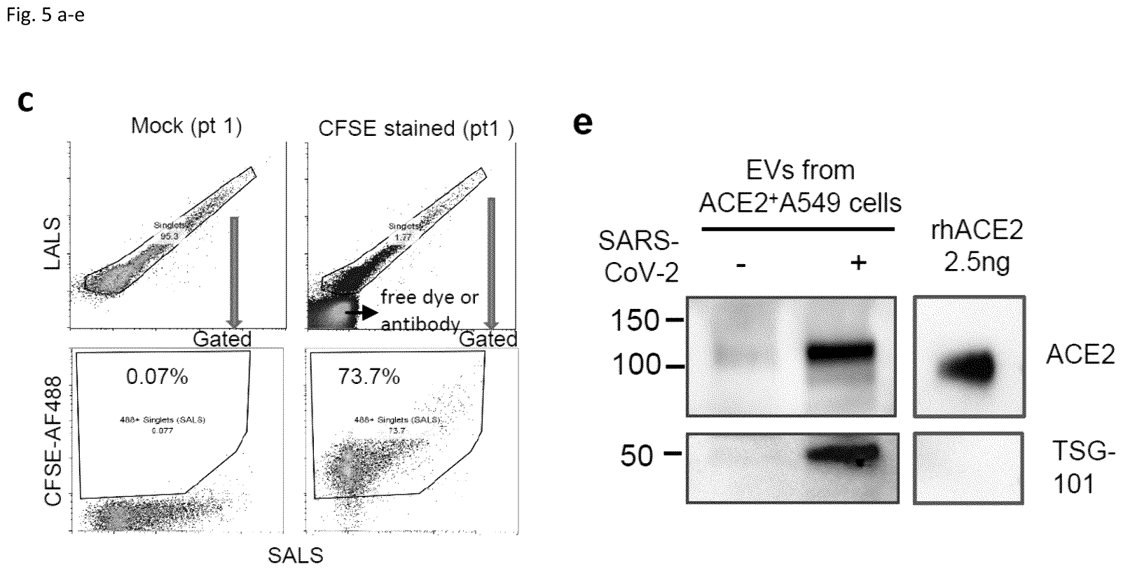

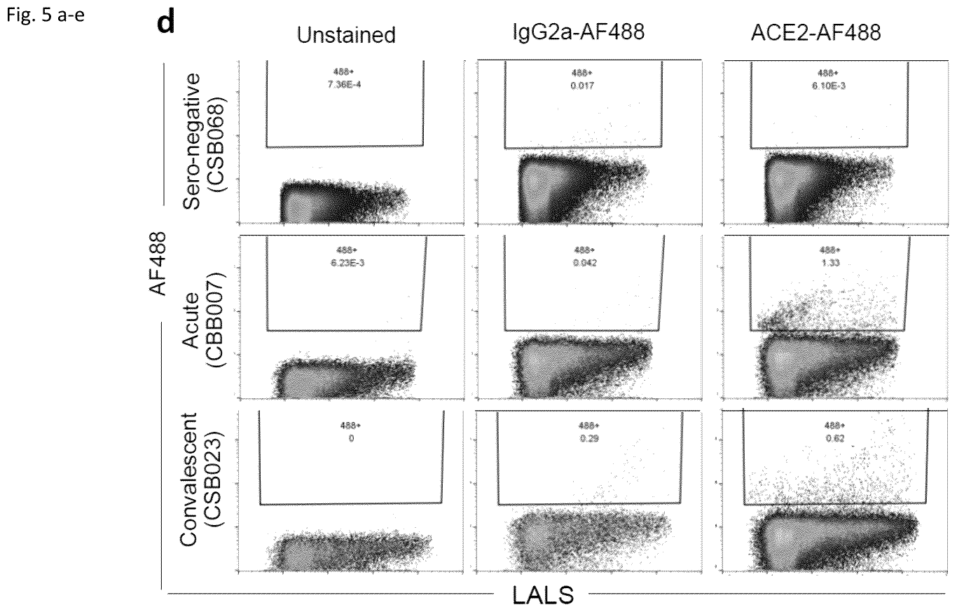

We previously established an automated and high throughput method, micro flow vesiclometry (MFV), to detect and profile the surface proteins of blood EVs at single particle resolution⁴³. Direct MFV analysis of circulating EVs in human plasma samples (Table 2, N=89) revealed elevated ACE2⁺ EVs in the plasma of COVID-19 patients in comparison to seronegative controls, with a more dramatic increase in the acute phase and a modest elevation in convalescent phase (FIG. 1a, b, FIG. 5a-d), with the latter in association with COVID-19 severe disease showing relatively higher levels in inpatient samples (FIG. 5b). ACE2⁺ EVs were enriched in CD63⁺ EV subsets from COVID-19 patients (FIG. 1c). Consistently, SARS-COV-2 infection triggered secretion of ACE2⁺TSG101⁺ EVs by human pneumocyte A549 cells overexpressing ACE2 (FIG. 5e), implying that upregulated production of ACE2+ EVs are part of the innate response to SARS-CoV-2 infection in COVID-19 patients.

TABLE 2
COVID-19 patient summary

| | Sero-negative | | CSB convalescent | | CBB acute phase cohort | |
|---|---|---|---|---|---|---|
| | (N = 5) | | (N = 61) | | (N = 23) | |
| | mean/count | SD/% | mean/count | SD/% | mean/count | SD/% |
| Age, years | 39.7 | 15.9 | 42.9 | 15.0 | 61.5 | 15.4 |
| Sex, male | 0 | 0.0% | 23 | 36.9% | 13 | 56.5% |

Race

| | | | | | | |
|---|---|---|---|---|---|---|
| Black | 0 | 0.0% | 6 | 9.4% | 9 | 39.1% |
| White | 3 | 60.0% | 37 | 57.8% | 9 | 39.1% |
| Asian | 0 | 0.0% | 4 | 6.3% | 1 | 4.4% |
| Other | 2 | 40.0% | 17 | 26.6% | 4 | 17.4% |
| SOFA score* | NA(N = 0) | NA | 6.0(N = 4) | 3.4 | 8.6(N = 14) | 4.1 |

Intermediate to advanced interventions

| | | | | | | |
|---|---|---|---|---|---|---|
| Vasopressor use | 0 | 0.0% | 4 | 6.3% | 10 | 43.5% |
| High flow nasal | 0 | 0.0% | 2 | 3.1% | 12 | 52.2% |
| cannula | | | | | | |
| Non-invasive | 0 | 0.0% | 1 | 1.6% | 3 | 13.0% |
| ventilation | | | | | | |
| Mechanical | 0 | 0.0% | 3 | 4.7% | 13 | 56.5% |
| ventilation | | | | | | |

Length of stay (LOS, days)

| | | | | | | |
|---|---|---|---|---|---|---|
| ICU patients | NA(N = 0) | NA | 22.4(N = 5) | 16.1 | 31.3(N = 15) | 16.7 |
| Inpatients | NA(N = 0) | NA | 5.1(N = 7) | 3.0 | 9.5(N = 8) | 5.7 |
| Onset to sampling, days | NA | NA | 87.5 | 47.8 | 12.9 | 9.4 |

Summary of sero-negative, acute, and convalescent phase of COVID-19 patients from which the plasma ACE2⁺ EVs and RBD-IgG levels were measured were measured.
*The SOFA score was calculated on ICU patients only. All patients are alive at the time of preparing this document.

In order to elucidate the specific functions of evACE2 in anti-SARS-COV-2 infection, we established a working protocol for characterizing ACE2 expression in EVs and determining the binding activity and neutralizing functions of evACE2 to SARS-COV-2.

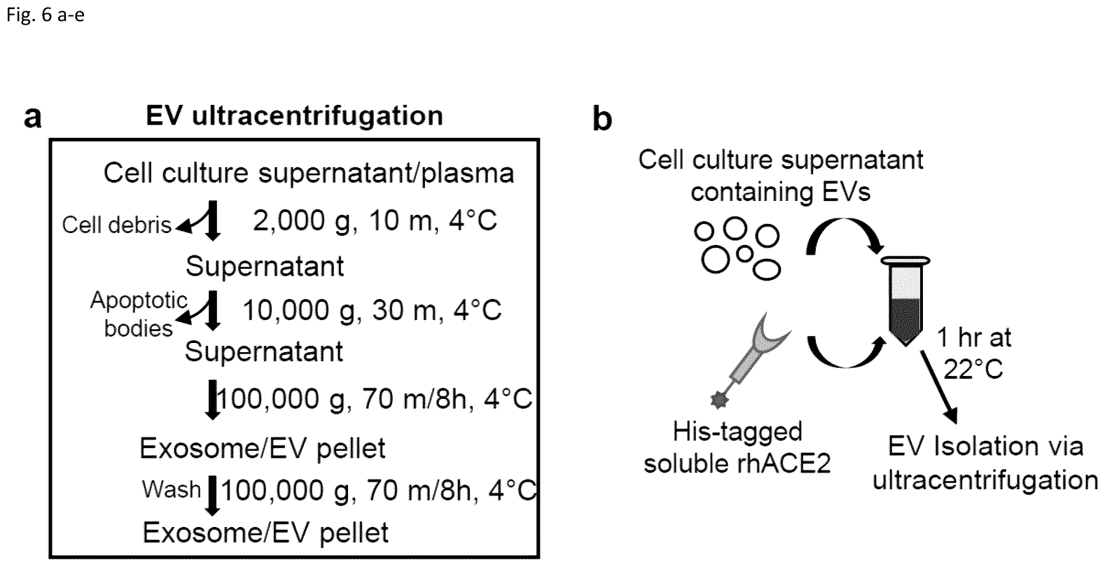

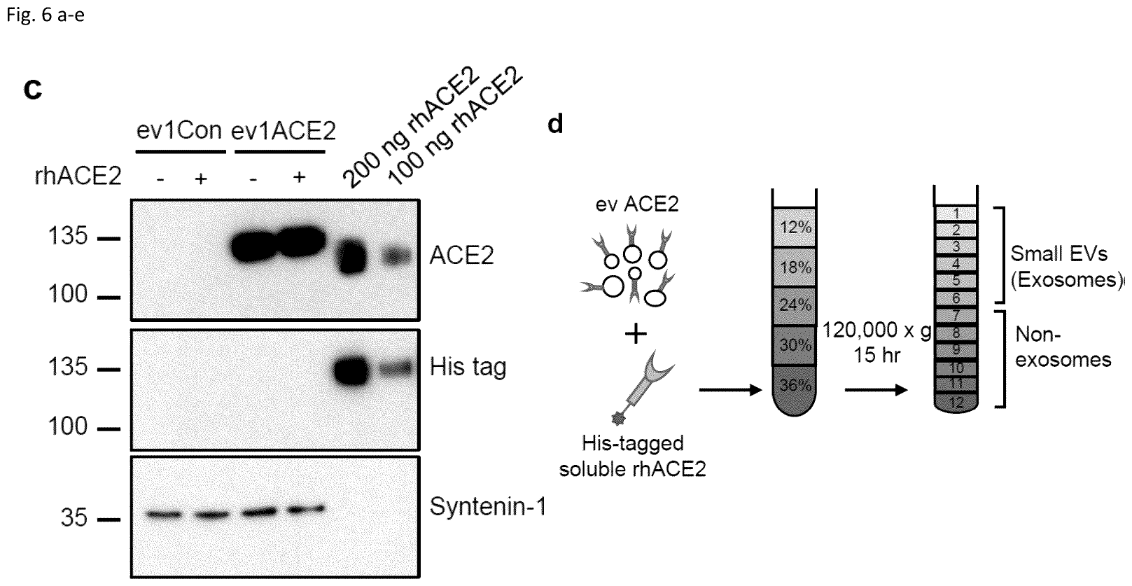

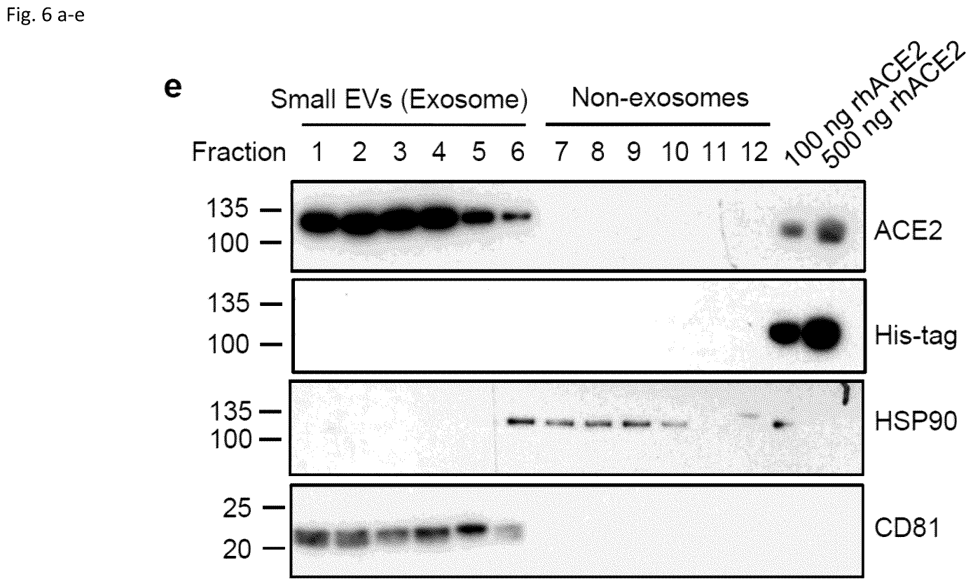

We used two sets of human cell lines HEK-293 (HEK) and HeLa, originally negative for ACE2 (ACE2-control), with stable expression of ACE2 (FIG. 1d). We then utilized a standard ultracentrifugation protocol (100,000 g×70 min) to isolate exosome-enriched EVs from the culture supernatants of these cells after removal of cell debris and apoptotic bodies (FIG. 6a). Using nanoparticle tracking analysis (NTA), EVs purified from ACE2⁺ and control cells exhibited a mean size of approximately 180-200 nm with equivalent vesicle counts of 6-8×10⁷ per μg of EV proteins (FIG. 1c). Immunoblotting demonstrated that the EVs from ACE2-expressing cells, but not the control EVs, were positive for ACE2 while both EVs displayed exosome-enriching markers CD63, CD81, TSG101, and Syntenin-1 (a newly identified high-abundance exosome marker⁵⁰), excluding the endoplasmic reticulum protein marker GRP94 (FIG. 1f, FIG. 6b-c). We also confirmed that evACE2 purification via ultracentrifugation and/or Optiprep density gradient fractionation did not detect any His-tagged soluble ACE2 (extracellular region) or cleaved ACE2 (which would have relatively smaller molecular weight compared to evACE2 that contains a transmembrane domain), when His-tagged recombinant human ACE2 extracellular domain (rhACE2) was spiked to the culture supernatant prior ultracentrifugation (FIG. 6b-c), or to precipitated EVs prior to Optiprep fractionation (FIG. 6d-e). Lack of detectable His-tagged rhACE2in purified EVs indicates that EV purification does not accumulate appreciable soluble ACE2. The full-length ACE2 was almost exclusively detected in exosome-enriched EV fractions co-expressing CD81, with minimal or no detectable ACE2 in the non-exosome fractions that express the putative exomere marker HSP90 (FIG. 6d-c). These results indicate that evACE2 is enriched in the exosomes with minimal detection in presumed exomeres.

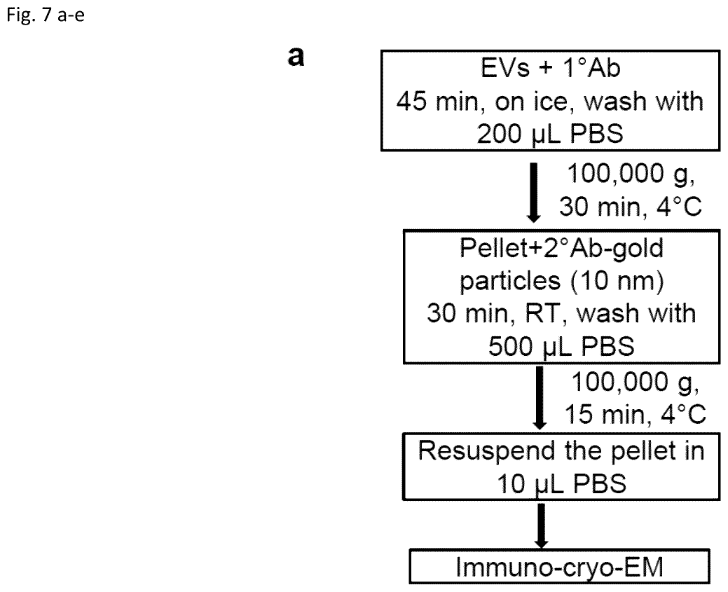

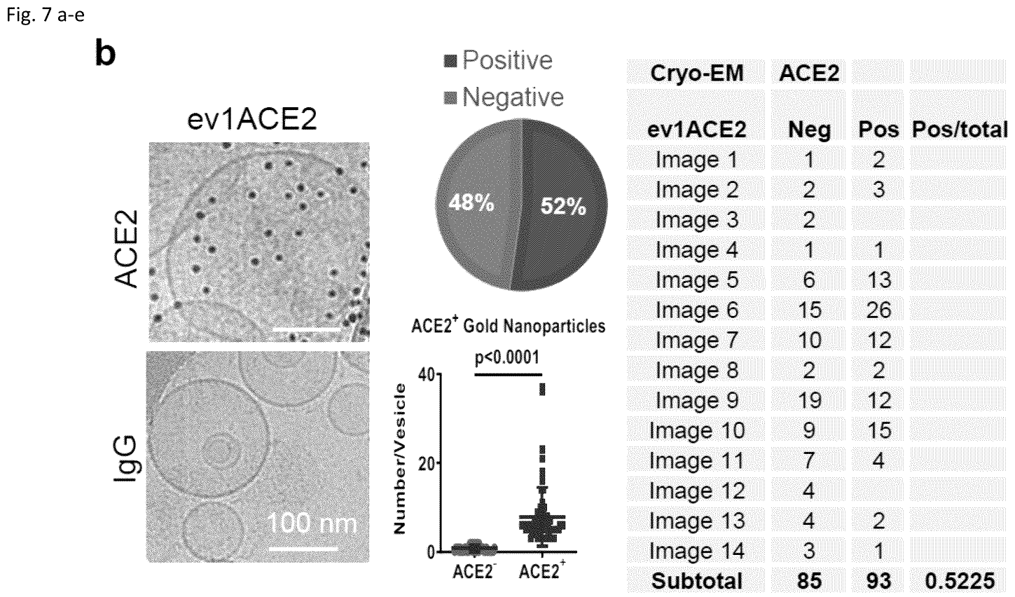

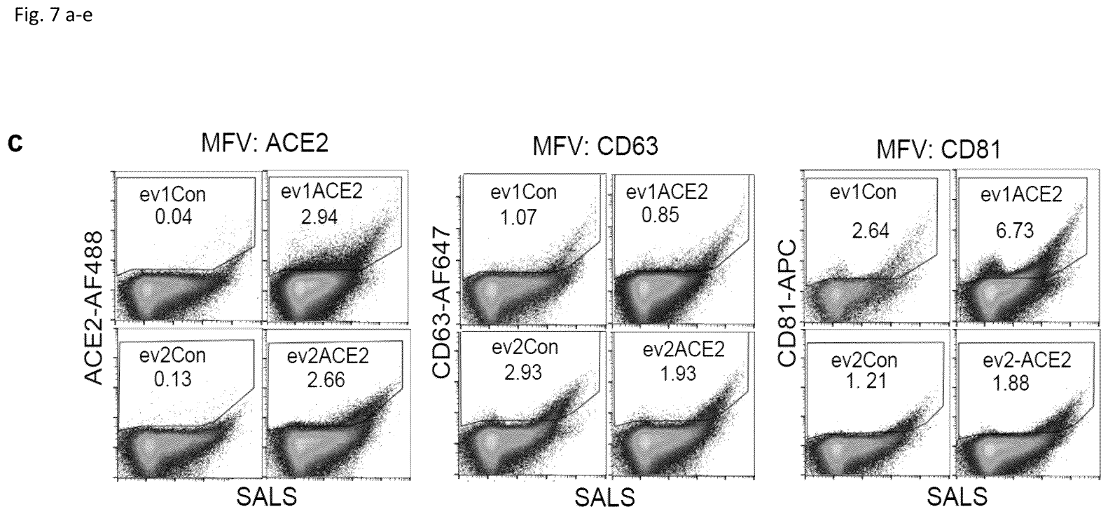

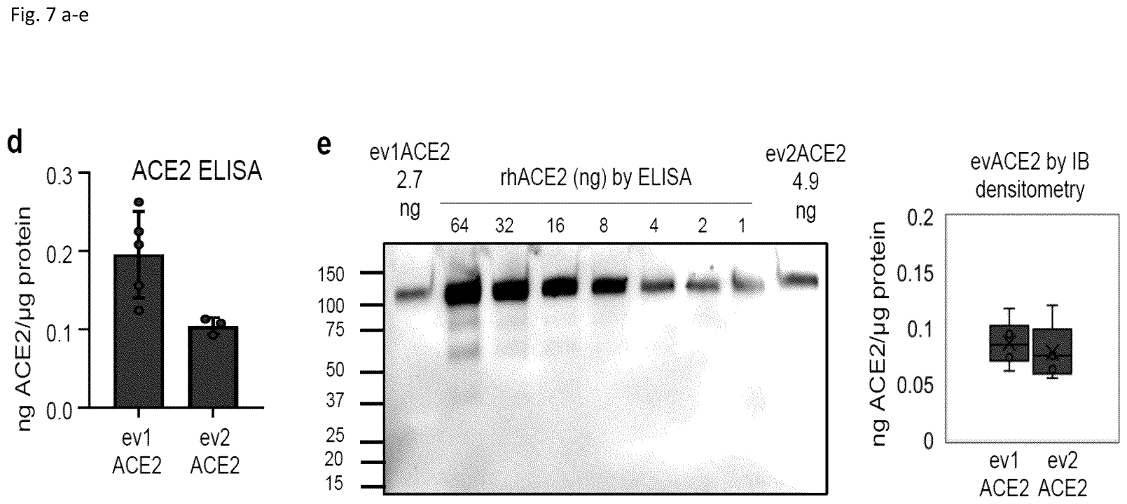

We further developed high-resolution cryogenic electron microscopy (cryo-EM) along with the high-throughput MFV to analyze ACE2 expression on EVs at single-particle resolution. Both methods detected ACE2 in the EVs derived from ACE2⁺ HEK and/or Hela cells, but not from their parental ACE2⁻ controls, whereas almost equivalent numbers of total EVs (0.5˜1×10⁸ counts per μg EV proteins) were produced from these cells (FIG. 1g-h, FIG. 7a-c), consistent with the NTA analyses. Immuno-cryo-EM revealed distinct expression of ACE2 (˜52%) in ACE2⁺ HEK cell-derived spheric EVs positive for CD81 (FIG. 7b). Double staining MFV analyses detected an enrichment of ACE2 in CD81⁺ EVs (31.9-62.5%) or CD63⁺ EVs (33.2-47.8%) for HEK-ev1 and HeLa-ev2 (FIG. 1h, i). We subsequently quantified the average ACE2 concentrations or molecular ratios in evACE2 utilizing ELISA and immunoblotting analyses with rhACE2 as a standard. Both methods detected a similar range of ACE2 content in the isolated EVs, including 0.1-0.2 ng ACE2 per μg EV protein of HEK-ev1 and HeLa-ev2 (EV protein measured in PBS via Nanodrop) (FIG. 7d-e). Based on the molecular weight of ACE2 and number of EV particles detected in isolated HEK-ev1 and HeLa-ev2 respectively, each EV might present 20-40 ACE2 molecules. Collectively, our results demonstrate that the SARS-COV-2 entry receptor ACE2 protein is expressed on EVs, most likely as a full-length transmembrane protein.

### evACE2 Blocks SARS-COV-2 RBD Binding and Variant Infections

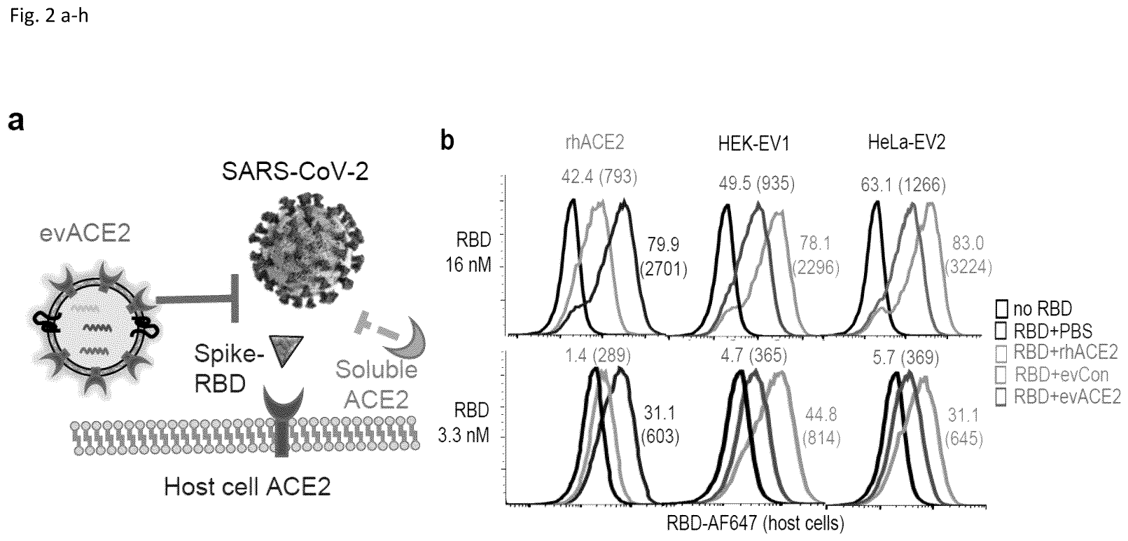

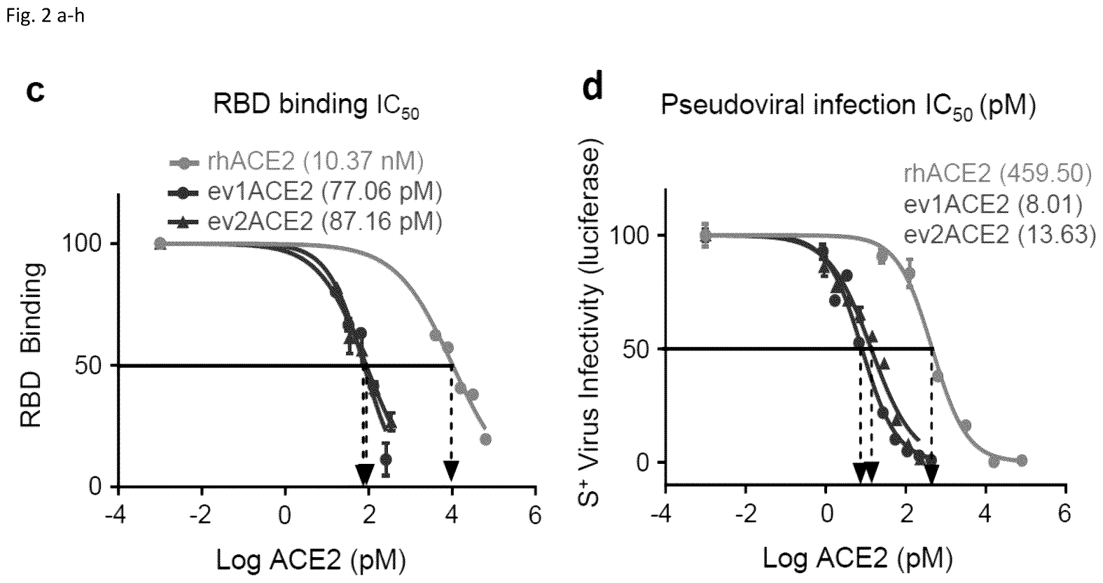

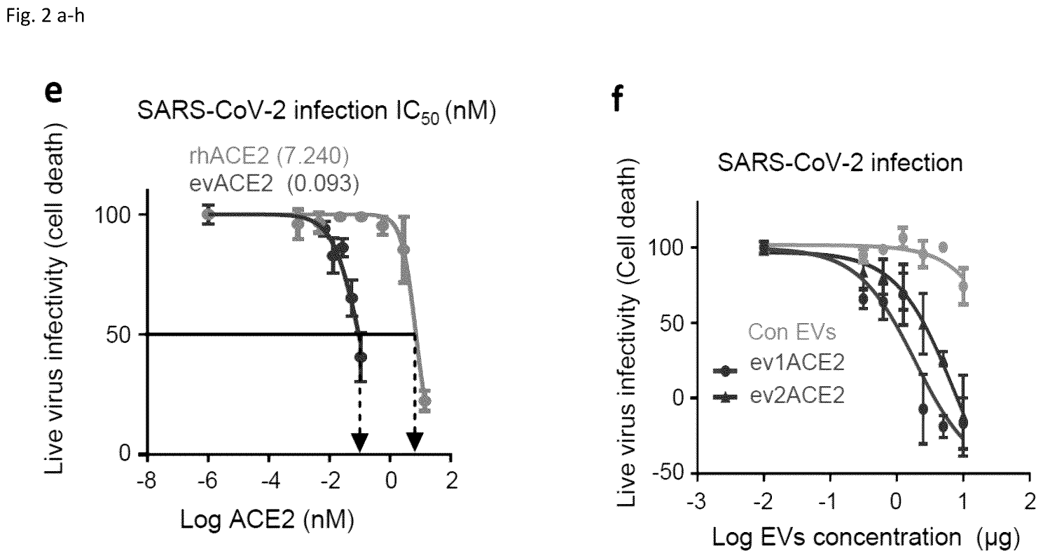

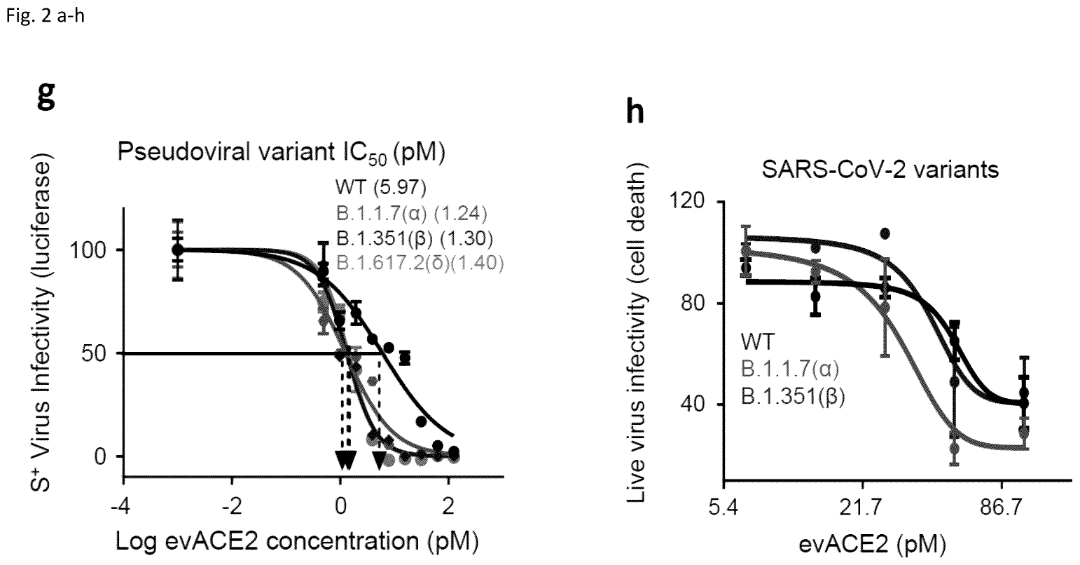

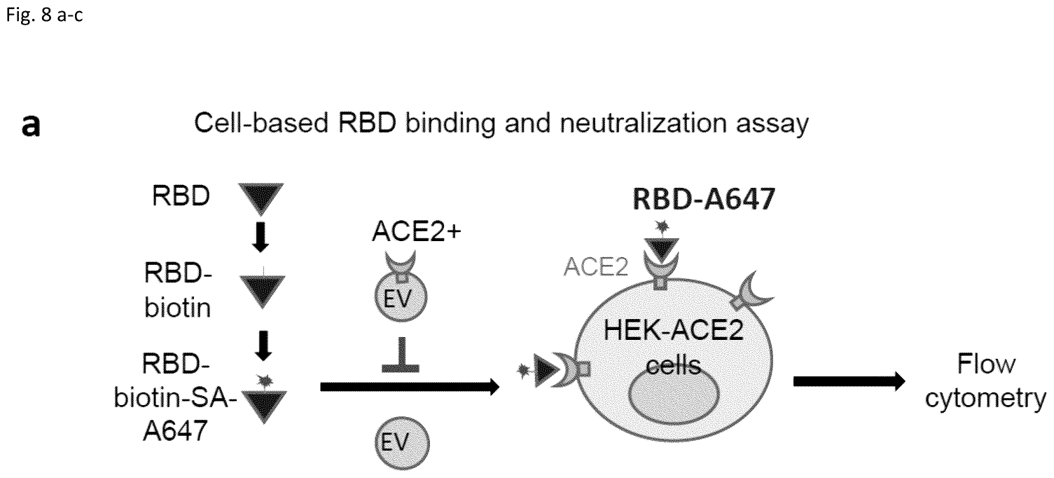

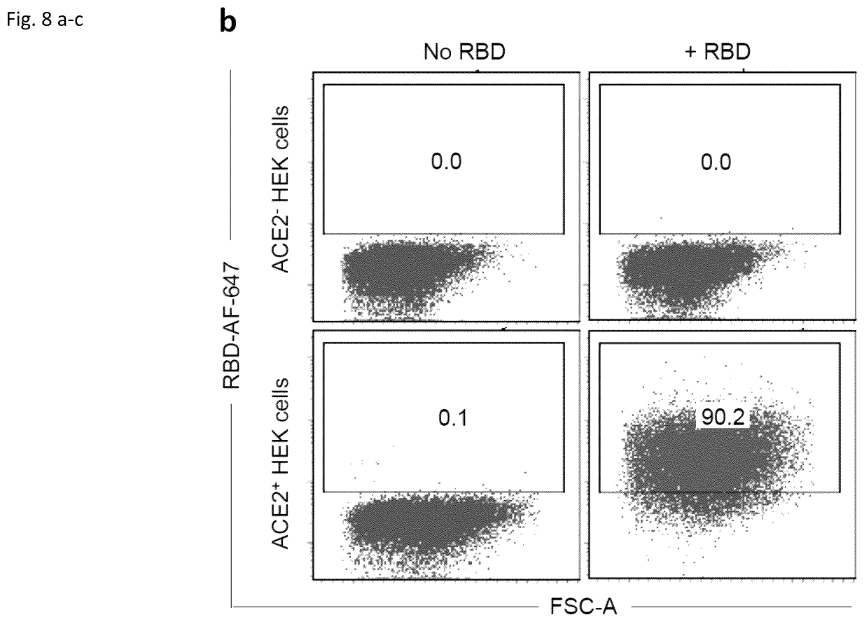

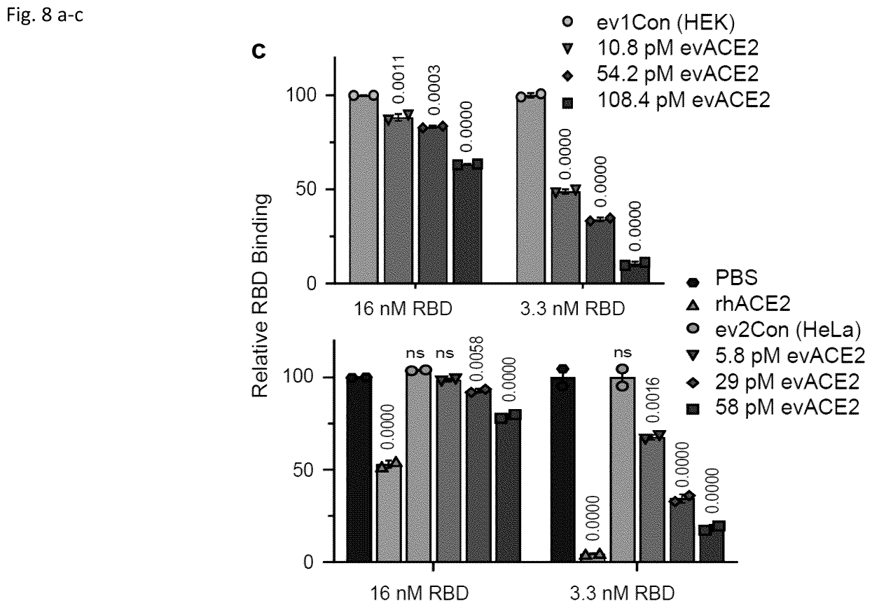

To analyze the effects of evACE2 on viral attachment and infection, we implemented a flow cytometry-based assay assessing the SARS-COV-2 S protein (RBD)-binding to human host cells (FIG. 2a, FIG. 8a). As expected, ACE2⁺ HEK cells displayed a specific and robust binding (>90%) with a red fluorophore AF-647-conjugated RBD protein (FIG. 8b). In a dose-dependent manner, the cell-bound RBD probe signals, both percentage of AF-647⁺ cells and the mean fluorescence intensity (MFI) were significantly inhibited by pre-incubation with 5 μg of ACE2⁺ EVs (0.5-1.0 ng evACE2) (FIG. 2b, c, FIG. 8c). In contrast, an equal amount of ACE2⁻EVs (5 μg) had negligible effects (FIG. 8c), indicating that the ACE2⁺ EVs inhibit SARS-COV-2 RBD recognition with their cellular receptor ACE2 through decoy ACE2 on EVs. As a positive control, rhACE2^{26,29} (140 ng) also inhibited the RBD binding to human ACE2⁺ cells (FIG. 2b, FIG. 8c).). Based upon evACE2 and rhACE2 serial dilution-mediated RBD neutralization assays, the IC⁵⁰ values of evACE2 are 77.06 and 87.16 pM for ev1ACE2 and ev2ACE2 from ACE2 overexpressing HEK cells and Hela cells, respectively, whereas the IC₅₀ for soluble rhACE2 to inhibit RBD binding to host cells is 10.37 nM (FIG. 2c). Therefore, evACE2 possesses 120-135 times more efficient blocking of SARS-COV-2 viral RBD binding to human host cells than soluble rhACE2.

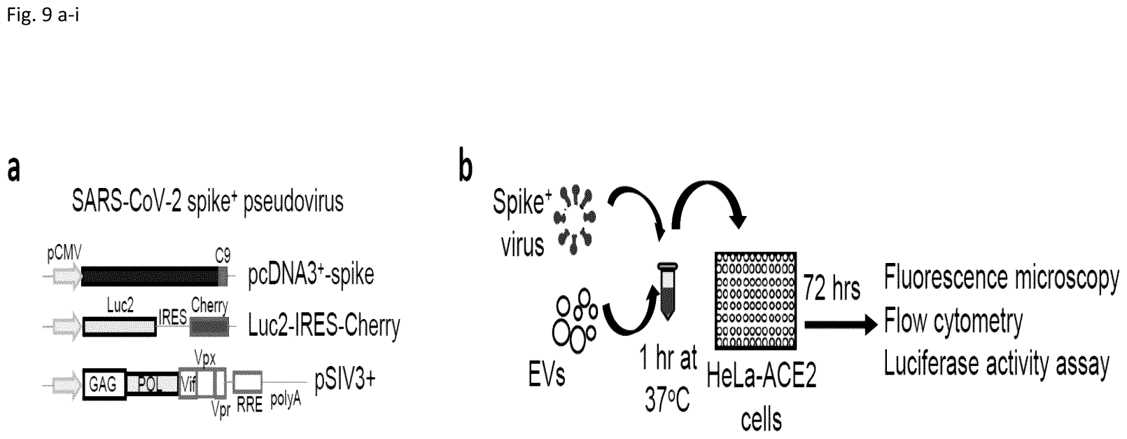

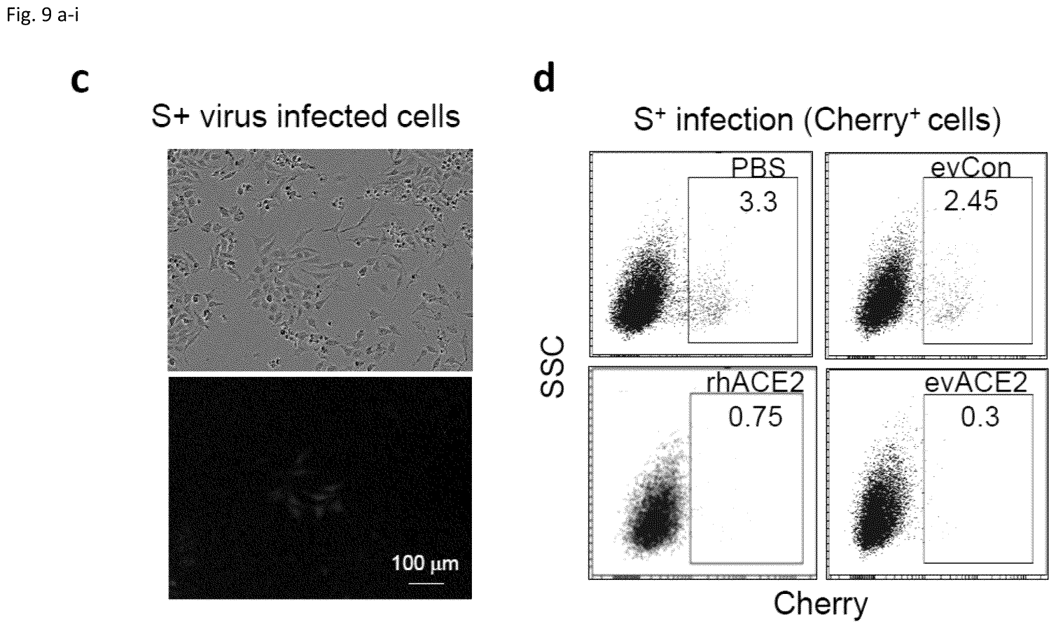

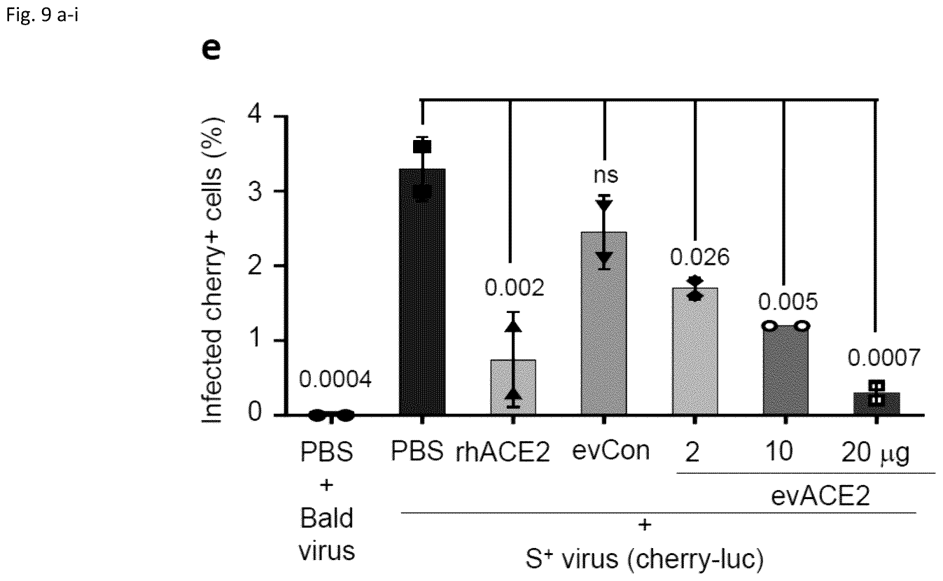

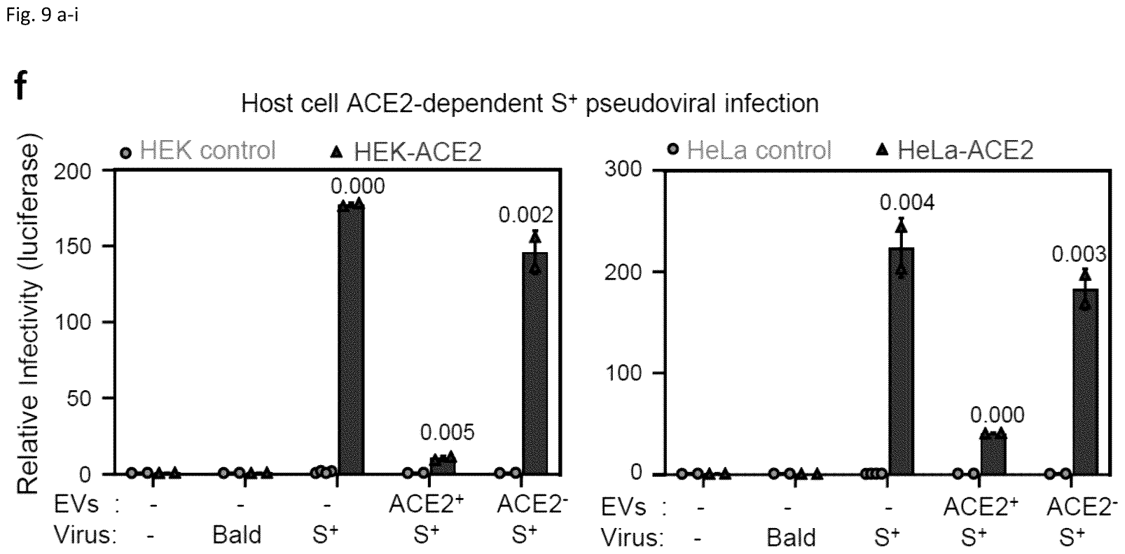

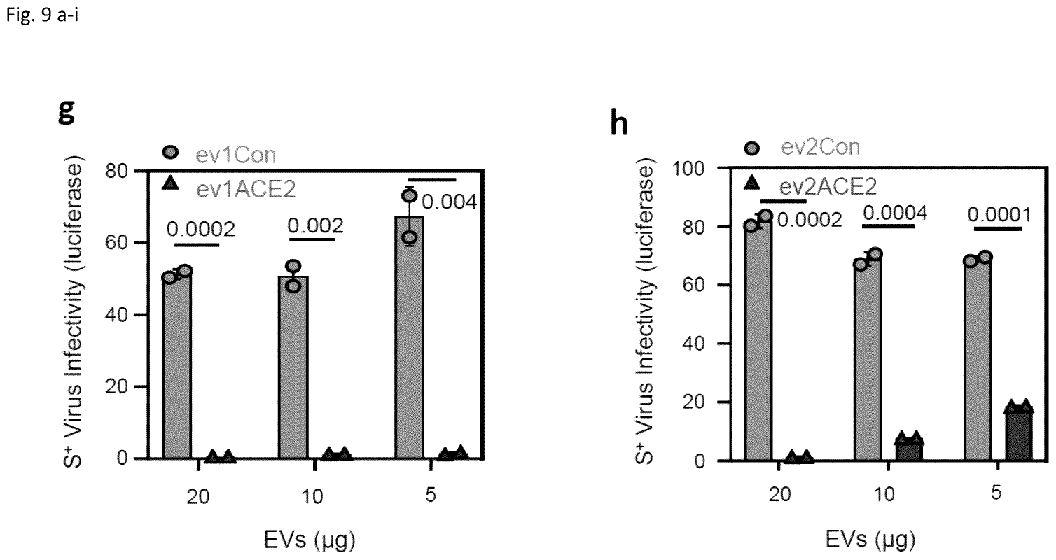

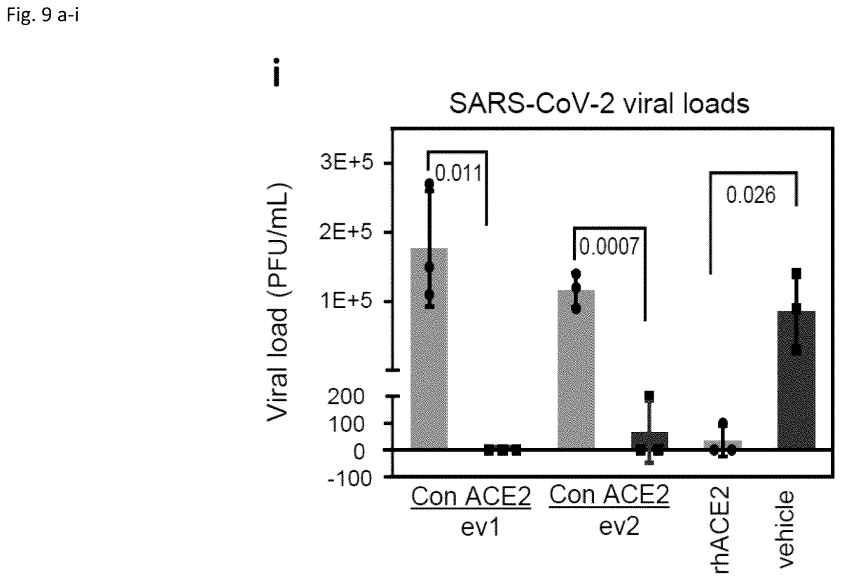

Next, we evaluated the neutralization effects of evACE2 and rhACE2 on the infectivity by SARS-COV-2 and its variants. When the SIV3-derived SARS-COV-2 S⁺ pseudovirus with either a dual Luc2-IRES-Cherry reporter or a luciferase protein reporter was utilized, ACE2⁺ EVs (ev1ACE2 and ev2ACE2), instead of ACE2⁻ control EVs, blocked SARS-COV-2 S⁺ pseudovirus infection in a dose-dependent manner as shown by flow cytometry of Cherry expressing cells or by luminescence signal of cellular luciferase activity (FIG. 2d, FIG. 9a-h). In comparison to an IC₅₀ of 459.50 pM for rhACE2, the IC50 values for ev1ACE2 (HEK) and ev2ACE2 (HeLa) were 8.01 pM and 13.63 pM, respectively, representing an estimated 58- and 34-fold higher neutralization efficacy in blocking pseudovirus infection compared to rhACE2 (FIG. 2d). Preincubation of SARS-CoV-2 S⁺ pseudovirus with ACE2⁺ EVs did not yield any infection of ACE2-negative cells given our experimental set up (FIG. 9f), limiting the possibility that ACE2⁺ EVs preincubation would promote SARS-COV-2 infection in ACE2⁻ cells.

We further demonstrated that upon wild-type SARS-COV-2 infection (400 plaque-forming units or pfu), the IC₅₀ doses for evACE2 (ev1 and ev2) was 41.92-93.65 pM (1.93-4.32 μg EV) in inhibiting the loss of viable Vero-6 cells with decreased viral loads, whereas ACE2⁻ EVs failed to protect the cells (FIG. 2e-f). In comparison to the IC₅₀ of rhACE2 at 7.24 nM, evACE2 achieves at least an estimated 80-fold neutralization efficacy to block SARS-COV-2 viral infections. Consistently, evACE2-mediated inhibition of SARS-COV-2 viral loads in infected cells were validated by PCR tests (FIG. 9i).

To examine the potential of evACE2 in neutralizing SARS-COV-2 variants, we utilized both pseudotyped and authentic SARS-COV-2 infection assays. Importantly, evACE2 achieved up to 4 to 5-fold greater efficacy in blocking the infection of pseudotyped SARS-COV-2 variants that express S protein mutants from B1.1.7 (α variant), B1.351 (β variant), and B.1.617.2 (δ variant) when compared to WT (FIG. 2g). Similar results were obtained in evACE2-mediated neutralization against authentic SARS-COV-2 variants, wherein evACE2 inhibited the infection by variants a and B to similar or greater efficacy than WT (FIG. 2h). Collectively, our results support the use of evACE2 as an innovative methodology to prevent or limit infection by a broad spectrum of SARS-CoV-2 viruses, including both WT and variant strains.

### ACE2+ EVs From Human Plasma Neutralize SARS-COV-2 Infection

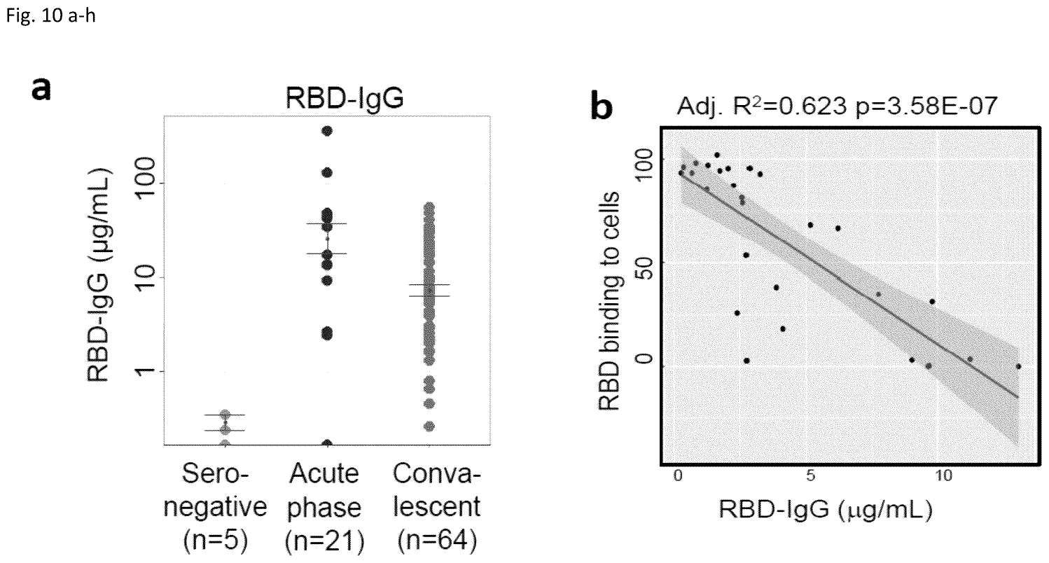

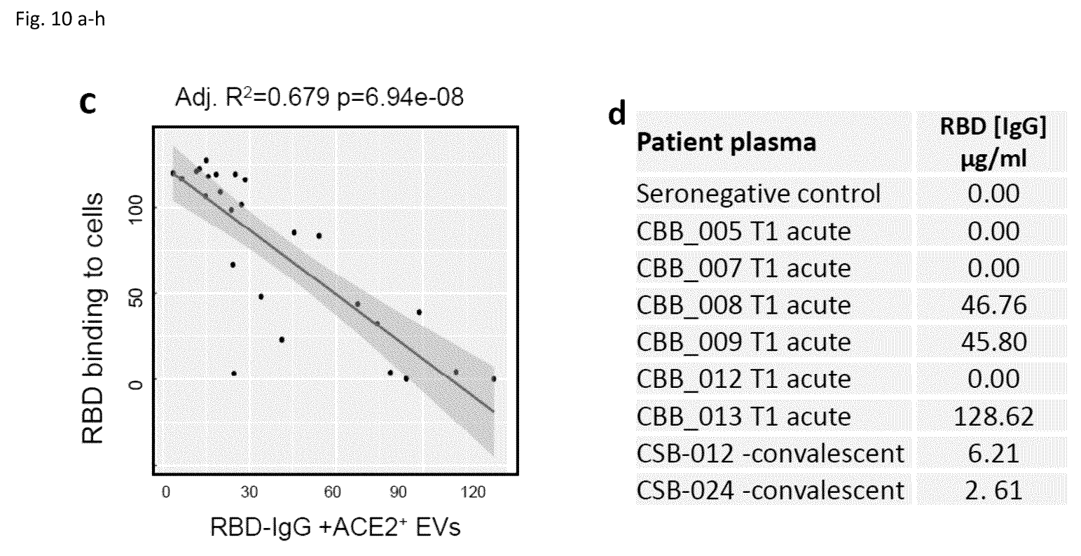

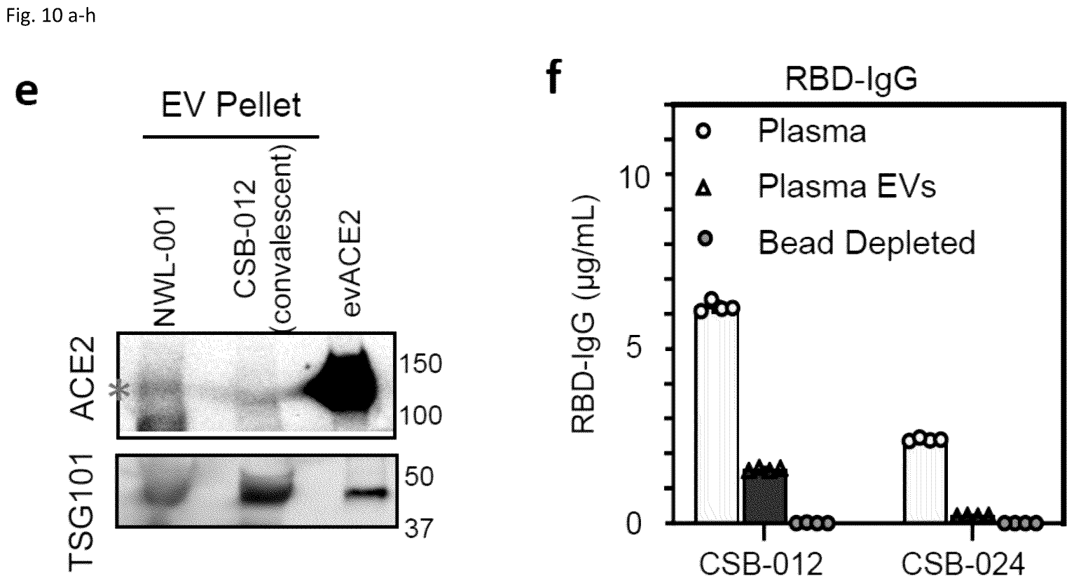

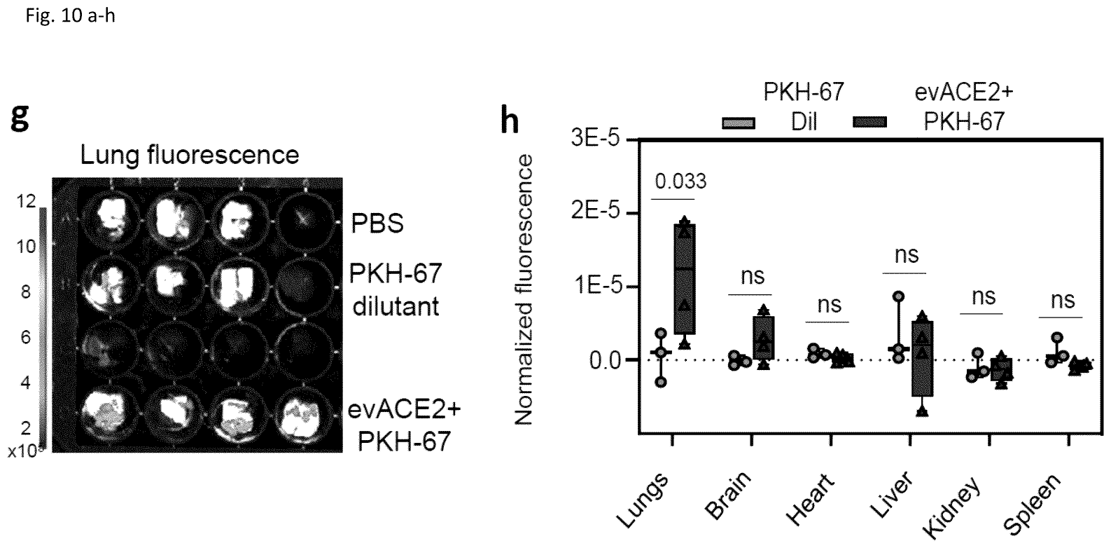

Our discoveries that circulating ACE2⁺ EVs are upregulated in the blood from COVID-19 patients and that the engineered ACE2⁺ EVs block SARS-COV-2 infection imply that evACE2 presents with a potential innate antiviral mechanism. We then investigated whether ACE2⁺ EV abundancy is associated with viral neutralization effect of human plasma. The elevated RBD-IgG levels in COVID-19 patient plasma were significantly associated with increased neutralization of RBD binding to human cells (FIG. 10a-b). Analysis of variance suggested that combining ACE2⁺ EVs levels with RBD-IgG levels, significantly improved the model fitting to explain the RBD neutralization activity of the tested plasma, with evACE2 accounting for 6.7% of the effects (p=0.027). The multivariable linear regression^{51, 52} of plasma neutralization activity on both ACE2⁺ EVs and RBD-IgG levels shows an improved R² of 0.679 (model's goodness-of-fit) than the RBD-IgG levels alone (FIG. 10c), supporting a potential antiviral contribution of circulating ACE2⁺ EVs.

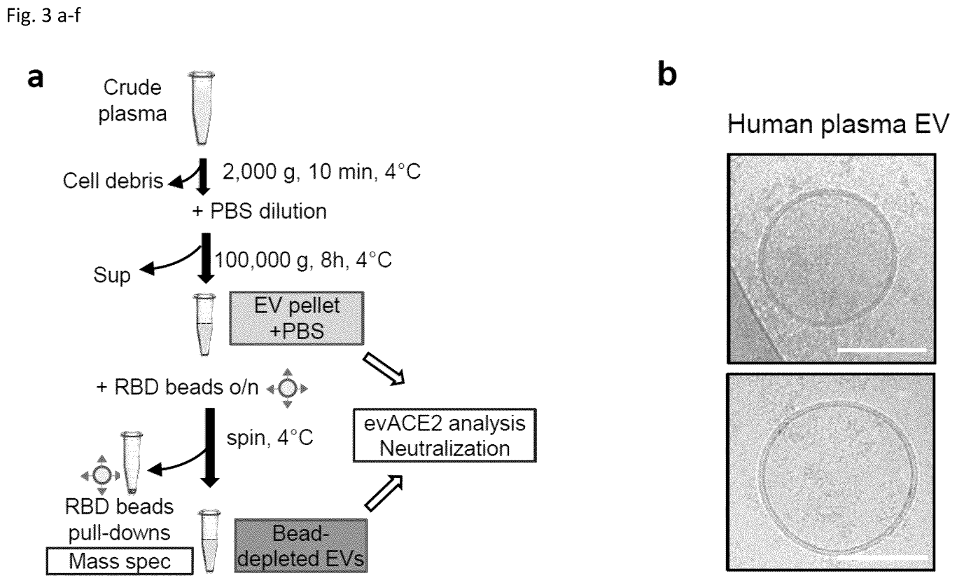

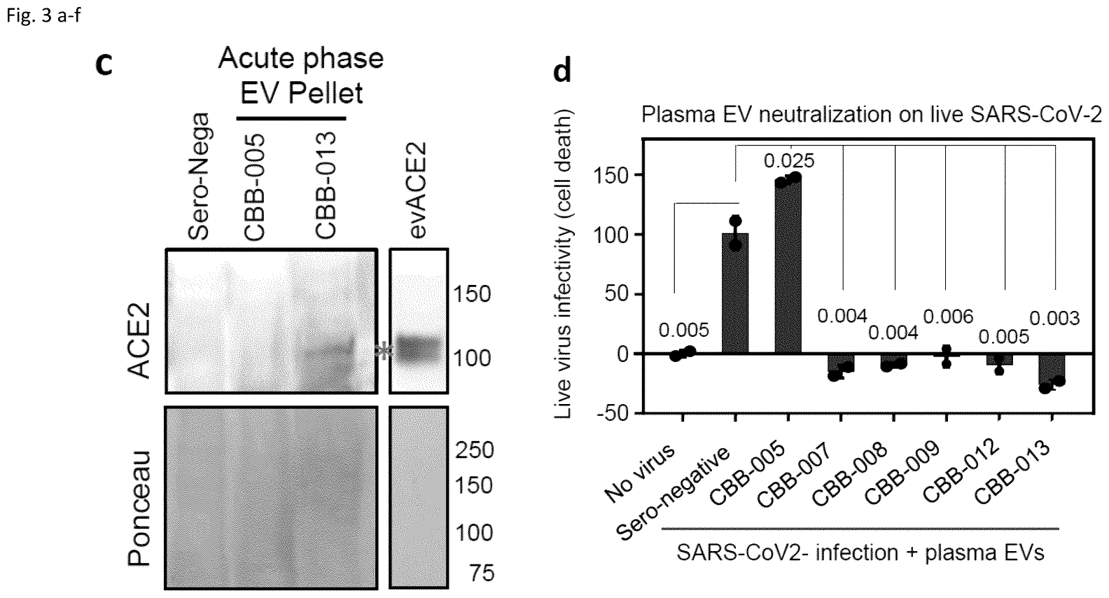

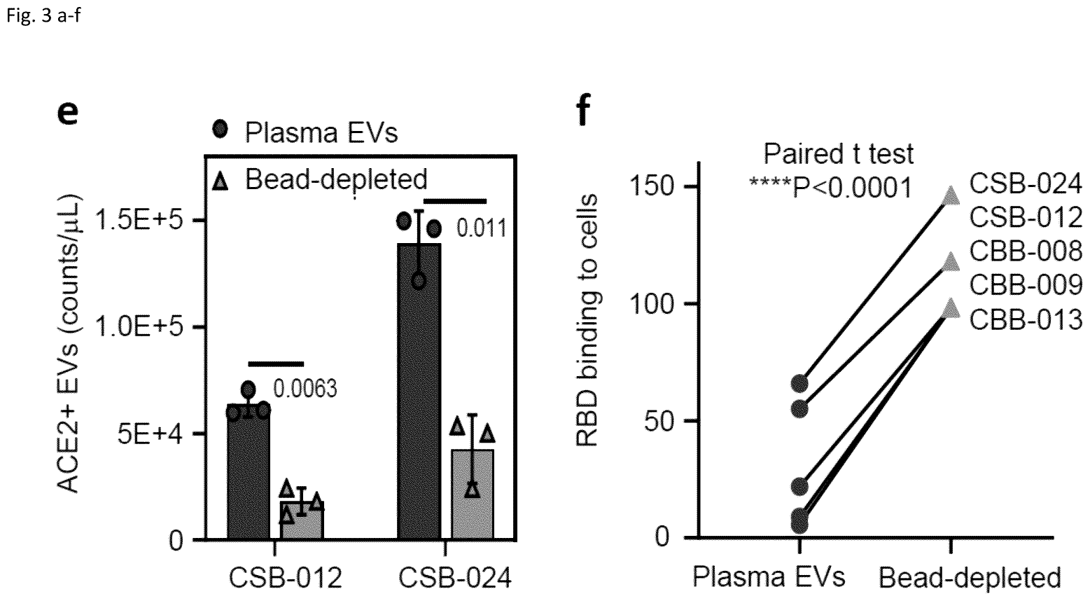

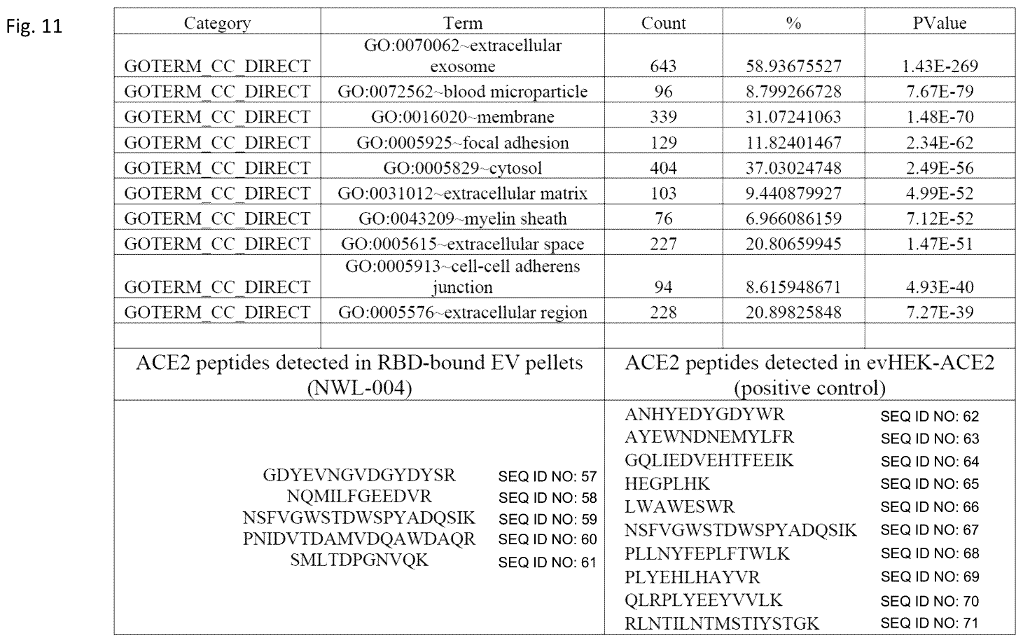

In order to determine the contribution of plasma EVs to neutralization functions, we isolated EVs from seronegative control and COVID-19 patient plasma samples, among which some samples had undetectable or low levels of RBD-IgG (FIG. 10d). Following the plasma dilution and extended ultracentrifugation, we detected EVs in the pellets using cryo-EM (FIG. 3a, b). The COVID-19 plasma pellet (acute phase CBB-013 and convalescent CSB-012) showed a transmembrane ACE2 band coupled with positive detection of an exosome marker TSG101 (FIG. 3c, FIG. 10c). Mass spectrometry analysis of the RBD-bead pull down materials from the patient plasma EV pellet confirmed the presence of ACE2 and EV proteins (FIG. 11).

Notably, the EV pellets isolated from the plasma of 5 of 6 acute phase COVID-19patients, especially CBB-007 and CBB-012 (no detectable RBD-IgG) completely blocked SARS-CoV-2 infection-caused cell death (FIG. 3d, FIG. 10d). In contrast, seronegative control and CBB-005 without detectable ACE2 EVs did not show any virus neutralization effects (FIG. 3c-d), suggesting that the plasma ACE2⁺ EVs levels, potentially regulated by SARS-COV2 infection, represent a previously unknown antiviral function in suppressing infection by SARS-COV-2.

We then used RBD-conjugated magnetic beads to deplete the majority of ACE2⁺ EVs in the plasma pellets (FIG. 3e), some of which had minimal or absent RBD-IgGs prior to and after depletion such as CSB-024 (FIG. 10f). Importantly, depletion of ACE₂₊ EVs isolated from in five plasma samples (convalescent CSB-012 and CSB-24, and acute phase CBB-008, 009 and 013) significantly impaired the ability of plasma EVs to neutralize RBD-binding to ACE2⁺ HEK cells (FIG. 3f), indicating that the ACE2⁺ EVs in the plasma from COVID-19 patients were at least partially responsible for anti-SARS-COV-2 activity.

### Intranasal evACE2 Protects hACE2 Mice From SARS-COV-2-Caused Mortality

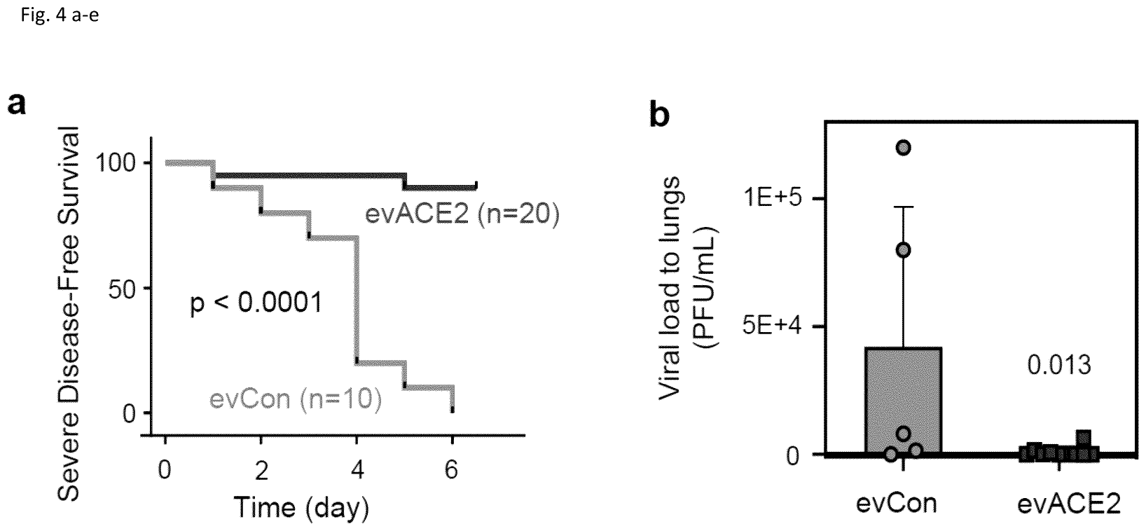

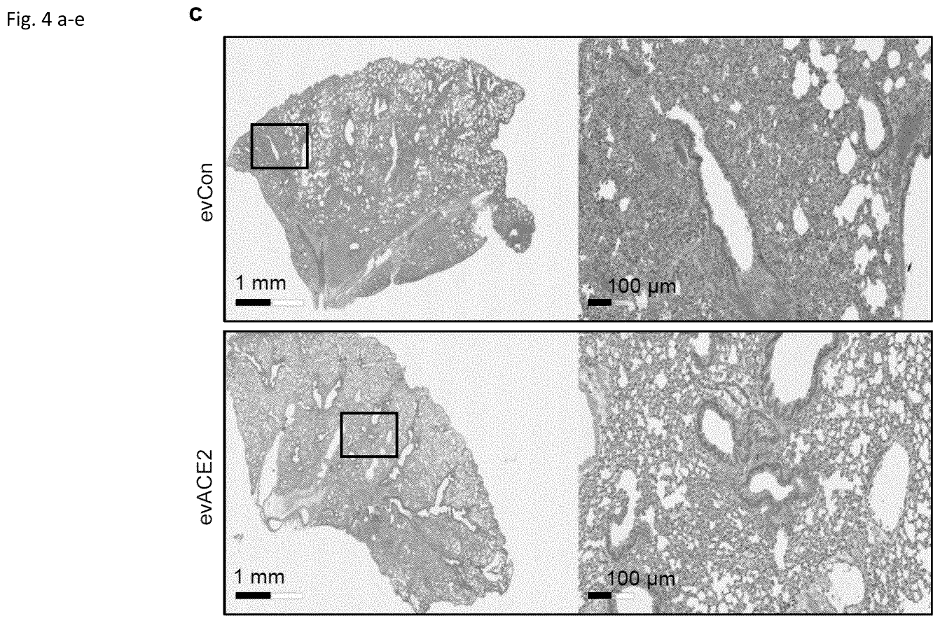

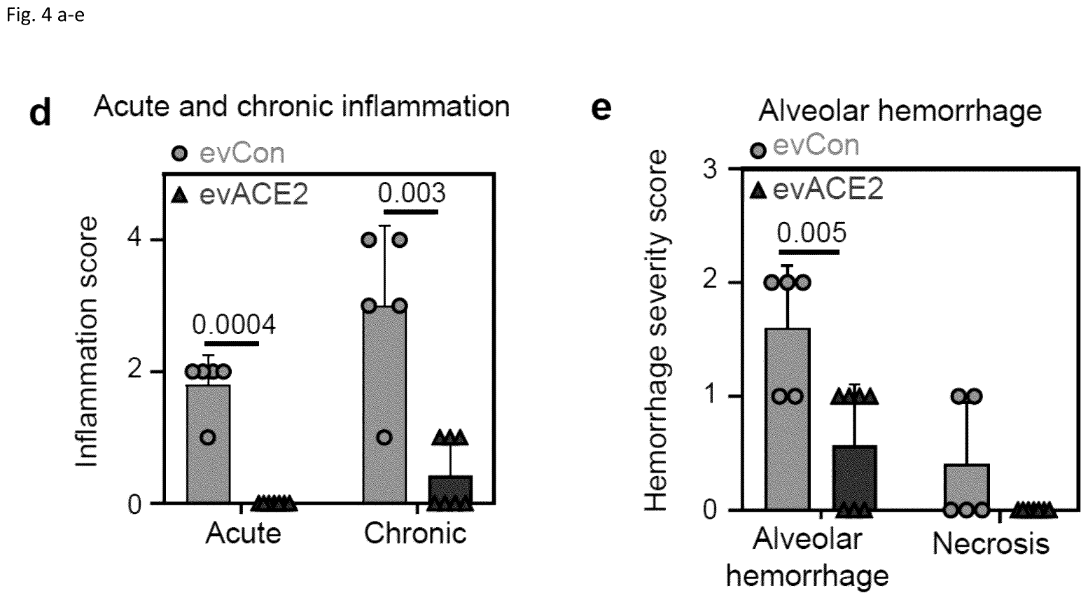

To further validate our discovery of evACE2 as a decoy therapy to treat COVID-19, we evaluated its preclinical therapeutic efficacy using a well-established hACE2 transgenic COVID-19 mouse model^{30,53,54}. In our study, the hACE2 transgenic mice showed acute weight loss 2-5 days following intranasal SARS-COV-2 infection (FIG. 4a). While recovery was often observed from day 6-7, and with a full recovery in about two weeks after a low dose of SARS-COV-2 infection, hACE2 mice had high mortality with a high dose of viral infection due to severe lung injury. Within a week after infection with 10,000 pfu of SARS-COV-2, nearly all mice succumbed (with 20% body weight loss, see methods) when treated with control EVs (FIG. 4a). Treatment with nasally delivered ACE2⁺ EVs (130 μg/mouse) significantly protected 80% of hACE2 mice from SARS-COV-2 infection-induced mortality (FIG. 4a). The protective activity of evACE2 was likely due to their inhibition of SARS-COV-2 infection of lung epithelia cells, given a more than 90% reduction in the SARS-COV-2 viral load detected in lung tissues from hACE2 mice treated with evACE2 compared to control EVs (FIG. 4b).

SARS-COV-2 infection of hACE2 mice resulted in lung injury that mimicked human COVID-19 pathogenesis, with a histopathology consisting of interstitial pneumonia with infiltration of considerable numbers of macrophages and lymphocytes into the alveolar interstitium, and accumulation of macrophages in alveolar cavities⁵⁵⁻⁵⁷. This COVID-19 lung pathogenesis was captured in H & E staining analysis of the lung tissue sections from the EV control group of hACE2 mice infected with SARS-COV-2 (FIG. 4c). Consistent with the reduced viral load in evACE2 treated mice, double-blind pathological scoring revealed that evACE2 treatment largely diminishes lung inflammation in the mice infected by SARS-COV-2 (FIG. 4d). Consequently, evACE2 treatment effectively protected hACE2 mice from SARS-COV-2 infection-mediated lung injury, with significantly reduced alveolar hemorrhage and necrosis scores compared to those in control EV-treated mice (FIG. 4e). To further validate whether the nasally delivered ACE2⁺ EVs are able to neutralize SARS-COV-2 in mouse lungs, we generated the fluorophore PKH67-labeled ACE2⁺ EVs and determined their biodistribution. Indeed, when PKH67-labeled ACE2⁺ EVs were nasally delivered at the therapeutic dosage, their biodistribution was mainly limited to the lungs for local therapy (FIG. 10g-h). These results clearly demonstrate that evACE2 achieves a favorable preclinical efficacy to treat COVID-19 pathogenesis.

### Discussion

Our studies have defined evACE2 as an innovative decoy therapeutic that efficiently block the infectious diseases caused by SARS-COV-2 and its variants of concern, and presumably all future emerging coronaviruses that utilize ACE2 as their initial tethering receptor. Mechanistically, evACE2 inhibits SARS-COV-2 infection by competing with host cell surface ACE2, which has been also speculated in a recent study showing that rhACE2 inhibits SARS-CoV-2 infection^{26,29}. Consistent with the fact that one EV can only carry a limited number of total protein molecules⁵⁸, our quantification analysis by cryo-EM, ELISA and immunoblotting estimated up to 20-40 ACE2 molecules per EV. Importantly, evACE2 possess an 80-fold better efficiency to block SARS-COV-2 infection than soluble rhACE2. Of note, it has been recently reported that the exosomal delivery of STINGa potentiates its uptake into dendritic cells compared with STINGa alone, which led to increased accumulation of activated CD8⁺ T-cells and an antitumor immune response59.

Almost all dominant SARS-COV-2 variants of concern harbor mutations in the RBD of S protein, such as N501Y (α and β) and E484K (β) that facilitate and strengthen the interaction between the virus and ACE2 receptor^{1,4,5,10-12}. The delta variant mutations not only result in an enhanced receptor binding, but also increase the rate of S protein cleavage, resulting in enhanced transmissibility⁶⁰. While such mutations render the variants resistant to vaccine-induced immunity and existing monoclonal antibody therapy, evACE2 can bind and neutralize these variants with an equal or even higher efficacy than for the WT strain, supporting their potential use as a broad-spectrum antiviral mechanism.

Without wishing to be bound by theory, we speculate the following two potential mechanisms underlying how evACE2 achieves its superior efficacy in blocking SARS-COV-2 infection than that of soluble ACE2 or soluble ACE2-conjugates^{61,62}: first, as small EVs are in an average size of 100-200 nm, proteins presented on small EVs might amplify the space interval in suppressing SARS-COV-2 access to its host cell surface. Second, it is also possible that EV expression may increase the affinity of ACE2 binding with the SARS-COV-2 S protein through synergy among ACE2 proteins on the same EV, and/or through the transmembrane domain which is involved in presenting an optimal ACE2 conformation for binding with S proteins.

Beyond a significant amplification of evACE2 in the anti-SARS-COV-2 efficacy in comparison to the purified rhACE2⁶³, we speculate that the therapeutic efficacy of evACE2 could be further potentiated through co-delivering additional anti-SARS-COV-2 medicines^{64,65}. This integration between surface ACE2 and antiviral medicine may allow us to develop superior therapies as well as to reduce the potential side effects from both therapeutics. EVs have been utilized as drug delivery systems with therapeutic potential against various disorders including infectious diseases and cancers^{44,66}. Of note, EVs derived from both plants and human specimen, such as dendritic cells and tumor cells, have been evaluated in multiple clinical trials and proven safe in humans₄₇₋₄₉. For example, cancer cell-derived EVs containing chemotherapeutic drugs, in addition to neo-antigens have been used to treat patients with malignant pleural effusion (NCT01854866 and NCT02657460). The EVs derived from plants, including grape (NCT01668849) and ginger or aloe (NCT03493984), have been registered in clinical trials in treating radiation- and chemotherapy-induced oral mucositis.

Circulating EVs in plasma represent an important component of blood in terms of their defensive, homeostatic, and signal transduction properties^{42,43,45,46}. Importantly, our discovery reveals that after SARS-COV-2 infection, a substantial amount of ACE2⁺ EVs present in human plasma can function as a previously unknown innate antiviral mechanism and a potent decoy to protect host cells from coronavirus infection. The levels of this innate antiviral evACE2 appear to be elevated by and in response to acute SARS-COV-2 infection as shown in the plasma from COVID-19 patients and in culture media of infected cells. The evACE2 upregulation in the blood appears to be sustained even during the recovery phase from COVID-19 patients with severe disease. It has been well established that the clinical disease severity may be positively associated with higher SARS-COV-2 virial load67, implying a possibility that either the virial pathogens, or their associated pathogenesis, induce the generation of evACE2. Future studies are needed to investigate the molecular mechanisms underlying the regulation of evACE2 production following SARS-COV2 infection.

### Methods

Human subject study and biosafety approvals. All research activities with human blood specimens of pre-COVID-19, sero-negative (healthy) donors, and acute and convalescent COVID-19 patients were implemented under NIH guidelines for human subject studies and the protocols approved by the Northwestern University Institutional Review Board (STU00205299 and #STU00212371) as well as the Institutional Biosafety Committee.

Animal study statement. Experiments with SARS-COV-2 were performed in biosafety level 3 (BSL3) and animal BSL3 (ABSL3) containment in accordance with the institutional guidelines following experimental protocol review and approval by the Institutional Biosafety Committee (IBC) and the Institutional Animal Care and Use Committee (IACUC) at the University of Chicago. EV biodistribution studies with B6 mice in the absence of viral infections were approved by the Institutional Animal Care and Use Committee (IACUC) at Northwestern University.

Cell culture. The parent ACE2⁻ human embryonic kidney HEK-293 cells (HEK) or human cervical cancer HeLa cells (HeLa) are transduced with lentiviral pDual-ACE2 expression vector for stable ACE2 expression and production of ACE2+ EVs. Dr. Daniel Batlle and Dr. Jan Wysocki generously provide the HEK-293 cells overexpressing ACE2 (HEK-ACE2). Dr. Thomas Gallagher of Stritch Medical School, Loyola University kindly provided HeLa and HeLa-ACE2 cells via the Hope group. ACE2− parent cell serve as negative controls in production of ACE2⁺ EVs in the culture. A549 cells overexpressing hACE2 and Vero6 cells were maintained in DMEM with 10% FBS, 1% penicillin/streptomycin and 1% non-essential amino acids. Cells were tested for mycoplasma contamination before culturing in all the laboratories. Cells were grown in Dulbecco's Modified Eagle's Medium (DMEM) supplemented with 10% (v/v) fetal bovine serum (FBS), 100 U/mL penicillin and 100 mg/mL streptomycin. FBS used to prepare complete media was EV-depleted by ultracentrifugation at 100,000×g for 16 h at 4° C.

Flow cytometry. Cells were blocked with mouse serum IgG (Sigma, 15381) for 10 min at room temperature and then incubated with specific antibodies; AF-647 mouse anti-human ACE2 (R&D systems, FAB9332R), AF-488 mouse anti-human ACE2 (R&D systems, FAB9333G) (0.4 μg/10⁶ cells), AF-647 isotype control mouse IgG2b (R&D systems, IC003R) or AF-488 isotype control mouse IgG2bAF488 (R&D systems, IC003G) for 45 min on ice, followed by washing twice with 2% EV-free FBS/PBS. Finally, the cells were diluted in 2% EV-free FBS/PBS and analyzed on a BD-LSR II flow cytometer (BD Biosciences).

Isolation of cell culture derived EVs. EVs were isolated from the cell culture supernatant of each of the four cell lines as described previously⁴³. Cells were cultured as monolayers for 48-72 h under an atmosphere of 5% CO₂ at 37° C. When cells reached confluency of approximately 80-90%, culture supernatant was collected, and EVs were isolated using differential centrifugation. First, the supernatant was centrifuged at 2,000×g for 10 min then at 10,000×g for 30 min to remove dead cells and cell debris. Next, the supernatant was ultracentrifuged for 70 min at 100,000×g using SW41 TI or SW32 Ti swinging bucket rotor (Thermo Fisher Sorvall wX+ 80 or Beckman Coulter Optima XE) to pellet the EVs. EVs were then washed by resuspension in 30 mL of sterile PBS (Hyclone, Utah, USA), and pelleted by ultracentrifugation for 70 h at 100,000×g. The EV pellet was resuspended in 100 μL PBS and stored at −80° C. The EV proteins in PBS were measured on Nanodrop in most of the experiments unless specified in certain immunoblotting experiments in FIG. 3c.

Spiked soluble ACE2 analysis in EV purification. EK293 and HEK293 cells overexpressing ACE2 and the parental cells negative for ACE2 were cultured for 48 h in DMEM with 10% exosome-depleted FBS and 1% penicillin-streptomycin. 30 mL of conditioned media was collected from the cells and spiked with 2 μg recombinant ACE2 protein (RayBiotech 23020165), followed by incubation at room temperature for 1 h. EVs were isolated from the media as described above.

Density gradient fractionation of EVs. EVs were ultracentrifugation-isolated as described above in Section “Isolation of cell culture-derived EVs”, resuspended in PBS, mixed with 2 μg recombinant ACE2 protein (RayBiotech 23020165), and subjected to density gradient fractionation as previously described 68. Resuspended pellets were loaded into 2.4 mL of 36% Optiprep (Sigma-Aldrich), followed by sequential layering of 2.4 mL of 30%, 24%, 18%, and 12% Optiprep on top. Samples were then ultracentrifuged at 120,000×g for 15 h at 4° C. in a SW41 Ti rotor (Beckman Coulter). 1 mL fractions were collected, with numbering starting from the top fraction, diluted with 11 mL of PBS, and washed at 120,000×g for 4 h at 4° C. in a SW41 Ti rotor. Fractions were then resuspended in the appropriate buffer for downstream analysis. Assuming all rhACE2 is recovered in each density fraction, approximately 166 ng of rhACE2 is present in each fraction.

Immunoblotting. In FIGS. 1f and 3c, cells and cell-derived EVs (HEK and HeLa) were lysed using RIPA buffer with protease inhibitor cocktail (Thermo Scientific, 1861279) (1:100 dilution) for 30 min on ice, then centrifuged for 15 min at 4° C. and 14,000 rpm. Protein was measured using Bradford protein assay (BioRad, 5000006), and 10-20 μg of cell-derived proteins and 2-8 μg of EV-derived proteins (equivalent to 20-80 μg EV proteins measured in PBS via Nanodrop) were denatured at 100° C. for 5 min and loaded to SDS-PAGE, then transferred to PVDF membranes that were incubated O/N with the primary antibodies Membranes were then washed, incubated with the corresponding horseradish (HRP)-conjugated antibodies, washed, and then developed using Pierce ECL2 solution (Thermo Fisher Scientific, 1896433A) (FIG. 1f). Human plasma and plasma-derived EV samples (resuspended in PBS) were lysed with Lacmmli buffer (Bio-Rad, 1610747) for 30 min on ice and processed as mentioned above.

In FIGS. 6c and e, EVs were lysed by urea buffer (8 M urea, 2.5% SDS) with PhosSTOP (Roche 04906845001) and Complete mini EDTA free protease inhibitor (Roche 11836170001) on ice for 30 min. EVs-derived proteins (equivalent to 500 μg total EV proteins as measured on Nanodrop in PBS prior to lysis, loaded per lane) and recombinant ACE2 proteins (100-500 ng loaded per lane, as described in the figures) were denatured with LDS sample buffer (Thermo Fisher NP0007) and DTT at 70° C. for 10 min, and loaded to a 4-12% precast polyacrylamide gels, then transferred to a PVDF membrane using the Trans-Blot Turbo transfer system (BioRad). Membranes were blocked with 5% non-fat dry milk at room temperature for 1 h and then incubated with primary antibodies (diluted in 2% BSA) at 4° C. overnight. Membranes were then washed three times with TBS-T (TBS with 0.01% Tween-20) and incubated with secondary antibodies (diluted in 2% non-fat dry milk) at room temperature for 1 h. After washing three times with TBS-T, the membranes were developed using West-Q Pico ECL reagent (GenDepot W3652020) or Pierce ECL reagent (Thermo Fisher, 32106). For FIG. 6c, blots were probed for ACE2, washed with TBST, then probed for syntenin-1. In FIG. 6e, blots were probed for ACE2, washed with TBST, followed by probing for CD81, or His-tag followed by HSP90.

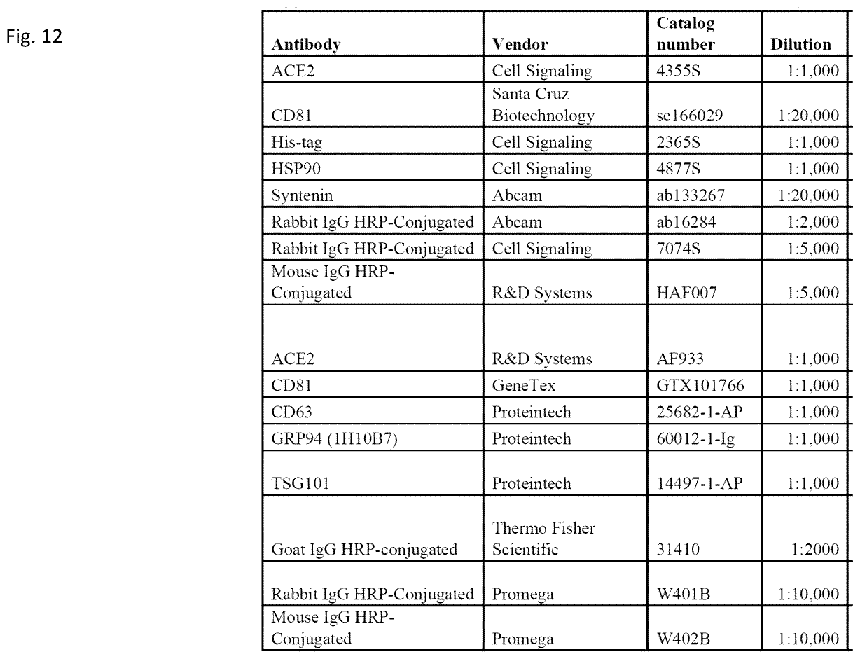

The antibody dilutions are shown in FIG. 12.

Detection of ACE2 in ACE2⁺EVs by ELISA and immunoblotting. EVs were isolated as mentioned above, lysed using RIPA buffer with protease inhibitor cocktail (1:100 dilution) for 45 min on ice, then centrifuged for 15 min at 4° C. and 14,000 rpm. First: Human ACE-2 ELISA kit (RayBiotech, ELH-ACE2-1) was used to detect ACE2. The antibody pair detects extracellular domain of Human ACE-2. The kit was used per manufacturer's instruction. Second: 27.3 and 87.5 μg of ACE2⁺ EVs (as measured in PBS by Nanodrop) were denatured at 100° C. for 5 min and loaded to SDS-PAGE, then transferred to nitrocellulose membranes that were incubated O/N with the ACE2 primary antibody (R&D systems, AF933). Membranes were then washed, incubated with the HRP-conjugated antibody, re-washed then detected by Pierce ECL2 solution. Image Lab was used for densitometry quantification (FIG. 7c).

Nanoparticle tracking analysis. Analysis was performed at the Analytical bioNanoTechnology Core Facility of the Simpson Querrey Institute at Northwestern University. All samples were diluted in PBS to a final volume of 1 ml and ideal measurement concentrations were found by pre-testing the ideal particle per frame value. Settings were according to the manufacturer's software manual (NanoSight NS3000).

Micro Flow Vesiclometry (MFV) Analysis of EVs. Antibody solutions were centrifuged at 14000×g for 1 h at 4° C. to remove aggregates before use. EVs (1-2 μg EV proteins, as measured on Nanodrop, in 20 μL of PBS) were blocked using 1 μg of mouse serum IgG for 10 min at RT then incubated with: AF-488 mouse anti-human ACE2 (R&D systems, FAB9333G), APC mouse antihuman CD81 (BD Biosciences, 561958), AF-647 mouse antihuman CD63 (BD, Biosciences, 561983), AF-488 isotype control mouse IgG2b (R&D systems, IC003G), APC isotype control mouse IgG₁κ (BD Biosciences, 555751) or AF-647 isotype control mouse IgG₁κ (BD, Biosciences, 557714) for 45 min at 4° C. The solution was then diluted to 200 μL with PBS and the samples were run on Apogee A50 Micro Flow Cytometer (MFC) (Apogee Flow Systems, Hertfordshire, UK) (http://www.apogeeflow.com/products.php). The reference ApogeeMix beads (Apogee Flow Systems, 1493), were used to assess the performance of Apogee MFC, and to compare the size distribution of the EVs. PBS was run as a background control.

Immuno-cryo-EM imaging. Antibody solutions and other staining buffers were centrifuged to remove non-specific particles or aggregates in the buffer of interest, at 14000×g for 1 h at 4° C. before use. EVs (10 μg in 100 μL PBS as measured on Nanodrop) were blocked using 5 μg of mouse serum IgG for 10 min at RT then incubated with mouse anti-human ACE2 (R&D systems, FAB9333G), mouse antihuman CD81 (BD Biosciences, 551108), isotype control mouse IgG2b (R&D systems, IC003G) or isotype control mouse IgG₁κ (BD Biosciences, 551954) for 45 min at 4° C. To rinse samples, 1 mL PBS was added to the tubes, and EVs were centrifuged 100,000×g for 30 min at 4° C. PBS was aspirated, samples were reconstituted in 100 μL PBS, and incubated with EM goat anti-mouse IgG (H&L) 10 nm gold conjugated (BBI solutions, EM.GMHL10) (7:100) for 30 min at RT. EVs were then rinsed by adding 1300 μL PBS then centrifugation 100,000×g for 15 min at 4° C. Finally, PBS was aspirated, and EVs were reconstituted in 50 μL PBS.

For cryoEM visualization, samples were prepared from freshly stained EVs at the concentration provided. For cryo-freezing, 3.5 μL of EV solutions were applied to fresh glow-discharged (10 s, 15 mA; Pelco EasiGlow) lacey carbon TEM grids (Electron Microscopy Services) and vitrified using a FEI Vitrobot Mark IV (FEI, Hillsboro, OR). The sample was applied to the grid and kept at 85% humidity and 10° C. After a 10 second incubation period the grid was blotted with Whatman 595 filter paper for 4 seconds using a blot force of 5 and plunge frozen into liquid ethane. Samples were imaged using a JEOL 3200FS electron microscope equipped with an omega energy filter operated at 200 kV with a K3 direct electron detector (Ametek) using the minimal dose system. The total dose for each movie was ˜20 e-/A2 and was fractionated into 14 frames at a nominal magnification between 8,000 to 15,000 (pixel size on the detector between 4.1 Å to 2.2 Å, respectively). After motion correction of the movies⁶⁹, EVs were identified manually using ImageJ⁷⁰. Two grids were prepared and imaged with 10-20 fields for each condition.

Development of the SARS-Cov-2 RBD “bait”. RBD of 223 amino acid (Arg319-Phe541) fragment of the SARS-COV-2 Spike protein that binds to the ACE2 receptor (Raybiotech, 230-30162-100) was biotinylated using NHS-PEG4-Biotin (Thermo Fisher, 21330). The protein was de-salted using Zeba Quick Spin columns (Thermo Fisher, 89849) and incubated with Streptavidin-AlexaFluor-647 (SA-AF-647) (Thermo Fisher, S21374) to make the RBD-biotin-AF647 bait. The RBD region of SARS-Cov-2 Spike is responsible for the initial step of coronavirus interaction with and attachment to human host cell receptor ACE2. While any viral antigen can be used as “bait” to identify virus-specific B cells and inhibitors, the RBD serves as one of the most powerful antigens and baits for identification of SARS-COV-2 vulnerable host cells, SARS-COV-2 specific B cells for producing RBD-neutralization antibodies (such as IgG), and other inhibitors to block coronavirus entry and infection. We have used the fluorophore-conjugated RBD bait: (1) for binding analysis of ACE2⁺ human host cells (HEK-293 and Hela) via flow cytometry, (2) for binding analysis of ACE2⁺ exosomes measured by micro flow vesiclometry, (3) for identification and sorting of SARS-COV-2 specific B cells with neutralizing IgG antibodies, and (4) for cell-based SARS-COV-2 neutralization tests to evaluate the capacity of ACE2⁺ exosomes, patient plasma, and IgG antibodies in inhibiting RBD binding to human host cells (HEK-293 and Hela).

Cell-based RBD binding neutralization by ACE2+ EVs and human plasma. The RBD-biotin-AF647 bait (3.3 and 16 nM) was incubated with EVs (ACE2⁺ and ACE2⁻), recombinant human ACE2 extracellular region (rhACE2, RayBiotech, 230-30165), or human plasma (10 μL or 80 μL) for 45 minutes on ice (creating “neutralized RBD”), then incubated with ACE2⁺ HEK-293 cells (200,000 cells in 100 μL 2% EV-free FBS/PBS) for 45 minutes on ice. Human recombinant ACE2 protein was used as a positive control (70-140 ng as determined by ELISA). RBD bait that was incubated with PBS, or with ACE2⁻ EVs, non-fluorescent RBD bait (mock control) and ACE2⁻ cells were used as controls. Cells were then spun and washed twice with PBS. DAPI was added as to exclude dead cells analyzed on flow cytometer and viable singlets were gated for percentage and mean fluorescence intensity (MFI) measurements of the RBD-AF647⁺ population.

Neutralization effects of ACE2⁺ EVs on SARS-COV-2 spike⁺ pseudovirus infection. The SARS-COV-2 spike (S⁺) pseudovirus carrying the Luc2-Cherry reporters were made for live virus neutralization assay after the pcDNA3-spike expression vector was transfected along with pCMV-Luc2-IRES-Cherry and pSIV3+ lentiviral vectors into a lentivirus producing cell HEK-293. Spike B.1.1.7 (α) variant (BPS Bioscience, 78112), B.1.351 (β) variant (BPS Bioscience, 78142) and Spike B.1.617.2 (β) variant (BPS Bioscience, 78215) pseudotyped lentivirus (Luc Reporter) were used. The S⁺ pseudovirus and/or variants were incubated with ACE2⁺ EVs, or ACE2⁻ EVs, or a positive control rhACE2, or negative control (PBS), for 1 h at 37° C. prior to the infection with ACE2⁺ human host cells HeLa in 96-well plates (5,000 cells/well). A bald virus without spike expression and ACE2⁻ cells served as negative controls. Flow cytometry of Cherry or eGFP and luciferase activity analysis (Promega, EL500) were used to assess viral infectivity.

Wild-type and variant SARS-COV-2 live virus infection to Vero-6 or A549 cells (BSL3). The wild-type SARS-COV-2 live virus study was conducted at the NIAID-supported BSL-3 facility at University of Chicago Howard T. Ricketts Regional Biocontainment Laboratory. One day prior to viral infections, 10,000 vero-6 cells were seeded per well in triplicates onto 96-well plates. 16 h after seeding, the attached cells were infected with mock controls (no virus) and wild-type SARS-COV-2 (400 pfu) viruses which were pre-mixed with a serial of doses of EVs (starting from 20 μg with 6 times of 1:2 dilutions) or an untreated control. 96 h later, the host cell viability (opposite to viral infectivity-caused cell death) was measured by crystal violet staining which stained attached viable cells on the plate following fixation. Cells killed off by the virus were floating and excluded. For the untreated control, the cells were infected but left without any treatment with a value of maximal cell death caused by the virus. The second control was the mock infected control where cells grew in the absences of virus or experimental sample representing the maximum normal cell growth over the time-period. The absorbance value of the untreated control was subtracted from all other absorbance values, thereby setting untreated wells to “0”, then all absorbance values were divided by the mock infected value thereby making that value 100.

A549 cells overexpressing ACE2 (A549-hACE2) cells were seeded (25,000 cells/well) in 24 well plates. 16 h after seeding, the attached cells were infected with mock controls (no virus) and wild-type SARS-COV-2 (MOI 0.1). Media was collected after 72 h of infection and inactivated at 65° C. for 30 min then shipped to Northwestern University. Media was spun 2000×g for 10 min, then supernatant was ultracentrifuged 100,000×g overnight using SW41 TI swinging bucket rotor (Thermo Sorwall wX+ 80) for maximal enrichment of evACE2. Pellets were reconstituted in equal volumes of PBS, lysed in RIPA buffer with protease inhibitor cocktail (1:100 dilution) for 45 min on ice, then centrifuged for 15 min at 4° C. and 14,000 rpm. EVs were denatured at 100° C. for 5 min and loaded to SDS-PAGE, then transferred to nitrocellulose membranes that were incubated O/N with ACE2 (R&D systems, AF933) and TSG101 (Proteintech, 14497-1-AP) primary antibodies. Membranes were then washed, incubated with the HRP-conjugated antibody, re-washed then detected by Pierce ECL2 solution (FIG. 5c)

RBD-IgG quantitative ELISA assay. The ELISA protocol was established as previously described^{71,72} and used herein with the modification of using plasma instead of scrum. Plasma samples were diluted by half with PBS during RosetteSep human B cell processing (StemCell Technologies #15064), aliquoted, and stored at −80C until analysis. Plasma was run in quadruplicate and reported as the average. Results were normalized to the CR3022 antibody with known affinity to RBD of SARS-COV-2⁷³. Sample anti-RBD IgG concentration reported as μg/ml was calculated from the 4PL regression of the CR3022 calibration curve. A sample value >0.39 μg/ml CR3022 was considered seropositive.

Plasma EV enrichment by ultracentrifugation. Sero-negative and COVID-19 (CBB at acute phase and CSB at convalescent phase) patient derived plasma samples were obtained from Northwestern Memorial Hospital and stored at −80° C. Frozen samples were thawed on ice, centrifuged 800×g for 5 min at 4° C., and then 2000×g for 10 min at 4° C. to remove debris. Then the 1ml plasma supernatant was diluted with 11 mL PBS and ultra-centrifuged at 100,000×g for 8 h at 4° C. (Beckman Coulter Optima L-90K Ultracentrifuge or Thermo Fisher Sorvall wX+ 80, SW41 TI swinging bucket rotor) to isolate and enrich EVs in the pellets. After centrifugation, supernatants and plasma pellets were collected separately. Plasma pellets were resuspended in appropriate volumes of PBS and subject to one round washing and ultracentrifugation at 100,000×g for 8 h at 4° C. The levels of ACE2⁺ EVs in plasma samples were evaluated by MFV on Apogee and western blotting using EV marker TSG101 and ACE2. ACE2⁺ cell culture derived EVs were used as a positive control.

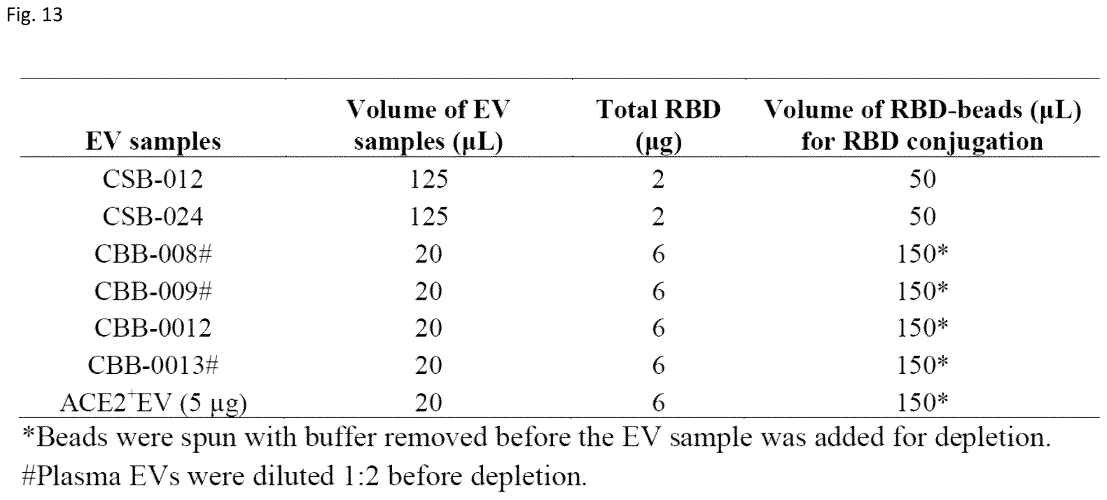

Depletion of ACE2⁺ EVs by RBD-conjugated beads. CSB and CBB patient plasma EVs were ultra-centrifuged above, and the EV-enriched pellets were resuspended in 250 μL PBS (per 1 mL plasma) for subsequent bead-mediated depletion. RBD-coupled magnetic beads or anti ACE2-coupled dynabeads beads were prepared according to manufacturer's protocols. Every 25 μL magnetic beads (CELLection Biotin Binder Kit, Thermo Fisher Sceintific, 11533D) were coupled with 1 μg of biotin-conjugated RBD protein (ACROBiosystems, SPD-C82E9). EV pellet samples were incubated with the beads for 30 min at 4° C. on rotator. And then the beads were removed by spinning or magnetic forces. The ratio of plasma samples and RBD-beads are shown in FIG. 13. Beads were removed by magnetic forces. The ACE2⁺ EV depletion efficiency was confirmed by MFV on Apogee and/or western blotting methods.

The altered neutralization effects of CSB and CBB plasma-derived EVs (resuspended pellets) prior to and after bead depletion were measured via flow cytometry as modified RBD binding to human host cells as described above. And rhACE2 protein (RayBiotech, 230-30165) was used as a positive control (70-140 ng).

LC-MS/MS analysis of RBD-bead precipitated EVs and proteins. Proteins from RBD-beads precipitated fractions from plasma EV pellets (8 h ultracentrifugation of CSB-012, CSB-024, NWL-001, and NWL-004) and controls of rhACE2 protein and purified ev1ACE2 (from HEK-ACE2 cells) were resuspended in cell lysis buffer (12 mM SDC in 50 mM TEABC with 1% protease and phosphatase inhibitor, pH 8.0), reduced with 10 mM dithiothreitol for 1 h at 25° C., and subsequently alkylated with 10 mM iodoacetamide for 30 min at 25° C. in the dark. The SDC concentration was diluted 1:4 with 50 mM NH₄HCO₃ for enzymatic digestion. Proteins were digested with Lys-C (Wako) and sequencing-grade modified trypsin (Promega, V5117) at 25° C. for 14 h. After digestion, each sample was acidified, desalted, lyophilized, and reconstituted in 12 μL of 0.1% FA with 2% CAN. 5 μL of the resulting sample was analyzed by LC-MS/MS using an Orbitrap Fusion Lumos Tribrid Mass Spectrometer (Thermo Scientific) connected to a nanoACQUITY UPLC system (Waters Corp., Milford, MA) (buffer A: 0.1% FA with 3% ACN and buffer B: 0.1% FA in 90% ACN) as previously described⁷⁴. Peptides were separated by a gradient mixture with an analytical column (75 μm i.d.×20 cm) packed using 1.9-μm ReproSil C18 and with a column heater set at 50° C. Peptides were separated by a gradient mixture: 2-6% buffer B in 1 min, 6-30% buffer B in 84 min, 30-60% buffer B in 9 min, 60-90% buffer B in 1 min, and finally 90% buffer B for 5 min at 200 nL/min. Data were acquired in a data dependent mode with a full MS scan (m/z 400-1800) at a resolution of 120K with the AGC value set at 8×10⁵ and maximum ion injection at 100 ms. The isolation window for MS/MS was set at 1.5 m/z and optimal HCD fragmentation was performed at a normalized collision energy of 30% with AGC set as 1×10⁵ and a maximum ion injection time of 200 ms. The MS/MS spectra were acquired at a resolution of 60K. The dynamic exclusion time was set at 45 s. The raw MS/MS data were processed with MSFragger via FragPipe 75.76 with LFQ-MBR workflow. A peptide search was performed with full tryptic digestion (Trypsin) and allowed a maximum of two missed cleavages. Carbamidomethyl (C) was set as a fixed modification; acetylation (protein N-term) and oxidation (M) were set as variable modifications. The match-between function has been used based on 1% ion FDR. The final reports were then generated and filtered at 1% protein FDR. The results were shown in the FIG. 11. The information of spectral count, ion intensity, and Ion Count obtained via FragPipe was used for label-free quantitation. Mass spectroscopy raw data sets have been deposited in the Japan ProteOmeSTandard Repository⁷⁷ with accession numbers PXD029662 for ProteomeXchange⁷⁸ and JPST001379 for jPOST.

Patient association analyses. Circulating ACE2⁺ EV counts, RBD-IgG levels, plasma neutralization on RBD binding, and clinical data were collected from the laboratory and Northwestern EDW database, electronically recorded, and verified by laboratory staff. There were in total n=30 measurable data points for final statistical analyses. To reduce bias resulting from batch effects, four independent replications on RBD-IgG test were performed in the laboratory. Therefore, one-way ANOVA was performed to compare group means and it suggests that the replications did not show statistically significant batch or measure errors (F=0.01, p-value>0.9), thus, mean values of the replications were taken for analysis. In addition, log-linear model (Poisson regression) was fitted to estimate the associations between normalized percentage (%) of RBD binding to cells and independent predictors of interest. It suggested negative associations (sec FIG. 10b-c) and the adjusted R2 suggests that the combined circulating ACE2⁺ EV counts+RBD-IgG level explains the relation better than RBD-IgG alone (Adj. R²=0.623 p<0.0001). Following linear modeling to determine that combined ACE2⁺ EVs+RBD-IgG explains the relation better than RBD alone, relative importance of ACE2⁺ EVs as compared to anti-RBD IgG was calculated using the Lindeman, Merenda and Gold (1980)⁵¹ formula using the ‘relaimpo’ package in R (Grömping, 2006)⁵². Metrics were normalized to sum to 100%. Coeffecient from this analysis was used to create graphs in FIG. 10b-c. All statistical analyses were performed by R 4.0.2.

Animal experiments. Animal infection: 6-9 weeks old female and male B6.Cg-Tg (K18-ACE2) 2Prlmn/J (K18-hACE2) mice (Jackson Laboratory) were anesthetized by intraperitoneal injection with ketamine-xylazine (100 mg-20 mg/kg) prior to intranasal administration of EV and virus. The suspension of 1×10⁴ PFU of USA-WA1/2020 SARS-COV-2 (2019-nCOV, 10 μL) pre-incubated with EVs (130 μg in 20 μL) for 1 h at 37° C. was administered via drop pipetting into the right nostril of animals. Mice were monitored twice daily to record clinical symptoms and weighed daily for 6 days post-challenge with virus. Categories in clinical scoring included: Score 0 (pre-inoculation)—animal is bright, alert, active, with normal fur coat and posture; Score 1 (post-inoculation, pi)—animal is bright, alert, active, normal fur coat and posture, no weight loss; Score 1.5—animal has slightly ruffled fur but is active; weight loss under 2.5%; Score 2 (pi)—animal has ruffled fur, is less active; weight loss under 5%; Score 2.5 (pi)—animal has ruffled fur, is not active but moves when touched, may have hunched posture or difficulty breathing; weight loss 5-10%; Score 3 (pi)—same as score 2.5; weight loss 11-20%; Score 4 (pi)—animal has ruffled fur or is positioned on its side or back, dehydrated, has difficulty breathing; weight loss >20%; Score 5 (pi)—death. At day six post-challenge, all animals were euthanized and subjected to necropsy and lung dissection. For each animal, one side of the lungs was homogenized in 2% DMEM to infect Vero6 cells with serial dilutions for measurement of viral titers (plaque forming units, pfu), and the other side was fixed with 10% formalin for further histology studies.

Biodistribution experiment. B6 Mice used in the study were kept in specific pathogen-free facilities in the Animal Resources Center at Northwestern University. All animal procedures were complied with the NIH Guidelines for the Care and Use of Laboratory Animals and were approved by the respective Institutional Animal Care and Use Committees. EVs isolated from HEK-ACE2 cells (as described above), were stained with PKH67 (Sigma, PKH67GL-1KT) and PKH67 dilutant control prepared similarly without the presence of EVs. 6-9 weeks old female and male B6 mice (Jackson Laboratory) were deeply anesthetized using isoflurane, and the EV suspension in 25 μL was pipetted into the right nostril of animals. After 24 h, animals were sacrificed, and lungs, brain, heart, liver, kidney, and spleen were isolated, rinsed with PBS. The organs were then imaged, and total fluorescence efficiency quantified using IVIS imaging system.

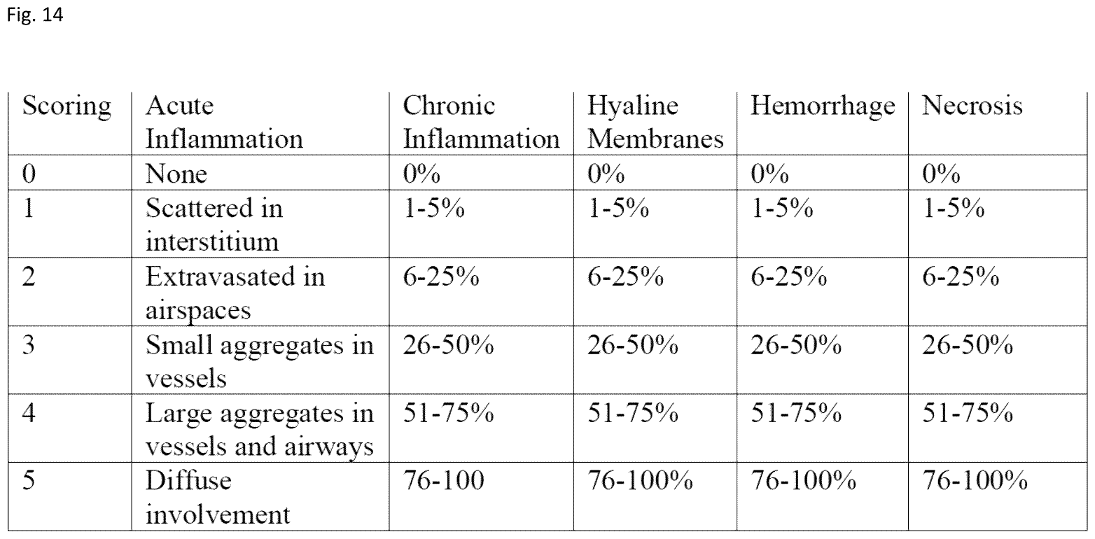

Histopathological analysis. Formalin-fixed and paraffin-embedded mouse lungs were processed, sectioned by routine procedures and the stained with H & E. Scoring was double-blinded and evaluated by a pathologist based on the percent of total lung surface area involvement (see FIG. 14), following the grading scheme adopted from a previous report⁷⁹.

Statistical Analysis. GraphPad Prism 9.0 Software was used to perform statistical analyses and calculate the IC₅₀. T-test or one way followed by Tukey posttest were used where appropriate (such as clear directions of changes). Results are significant if P<0.05. Data are presented as mean±standard deviation (SD). Parametric analysis was performed unless specified in the figure legends. Measurements were taken from distinct samples in all experiments with biological and/or technical replicates.

Data availability statement. Mass spectroscopy raw data sets have been deposited in the Japan ProteOmeSTandard Repository⁷⁷ (https://repository.jpostdb.org/). The accession numbers are PXD029662 for ProteomeXchange⁷⁸ and JPST001379 for jPOST. Other details are available upon request.

### REFERENCES

1 Zhou, P. et al. A pneumonia outbreak associated with a new coronavirus of probable bat origin. Nature 579, 270-273, doi:10.1038/s41586-020-2012-7 (2020).
2 Zhu, N. et al. A Novel Coronavirus from Patients with Pneumonia in China, 2019. The New England journal of medicine 382, 727-733, doi:10.1056/NEJMoa2001017 (2020).
3 Rothan, H. A. & Byrareddy, S. N. The epidemiology and pathogenesis of coronavirus disease (COVID-19) outbreak. J Autoimmun 109, 102433, doi:10.1016/j.jaut.2020.102433 (2020).
4 Korber, B. et al. Tracking Changes in SARS-COV-2 Spike: Evidence that D614G Increases Infectivity of the COVID-19 Virus. Cell 182, 812-827 c819, doi:10.1016/j.cell.2020.06.043 (2020).
5 Becerra-Flores, M. & Cardozo, T. SARS-COV-2 viral spike G614 mutation exhibits higher case fatality rate. Int J Clin Pract 74, e13525, doi:10.1111/ijcp.13525 (2020).
6 du Plessis, L. et al. Establishment and lineage dynamics of the SARS-COV-2 epidemic in the UK. Science, doi:10.1126/science.abf2946 (2021).
7 Lopez Bernal, J. et al. Effectiveness of Covid-19 Vaccines against the B. 1.617.2 (Delta) Variant. N Engl J Med 385, 585-594, doi:10.1056/NEJMoa2108891 (2021).
8 Wang, P. et al. Antibody resistance of SARS-COV-2 variants B.1.351 and B.1.1.7.Nature 593, 130-135, doi:10.1038/s41586-021-03398-2 (2021).
9 Li, W. et al. Bats are natural reservoirs of SARS-like coronaviruses. Science 310, 676-679, doi:10.1126/science.1118391 (2005).
10 Hoffmann, M. et al. SARS-COV-2 Cell Entry Depends on ACE2 and TMPRSS2 and Is Blocked by a Clinically Proven Protease Inhibitor. Cell, doi:10.1016/j.cell.2020.02.052 (2020).
11 Wrapp, D. et al. Cryo-EM structure of the 2019-nCOV spike in the prefusion conformation. Science (New York, N.Y 367, 1260-1263, doi:10.1126/science.abb2507 (2020).
12 Yan, R. et al. Structural basis for the recognition of the SARS-COV-2 by full-length human ACE2. Science (New York, N.Y, doi:10.1126/science.abb2762 (2020).
13 Mullard, A. First antibody against COVID-19 spike protein enters phase I. Nat Rev Drug Discov 19, 435, doi:10.1038/d41573-020-00108-x (2020).
14 Bost, P. et al. Host-Viral Infection Maps Reveal Signatures of Severe COVID-19 Patients. Cell 181, 1475-1488 e1412, doi:10.1016/j.cell.2020.05.006 (2020).
Chi, X. et al. A neutralizing human antibody binds to the N-terminal domain of the Spike protein of SARS-COV-2. Science (New York, N.Y, doi:10.1126/science.abc6952 (2020).
16 Baum, A. et al. Antibody cocktail to SARS-COV-2 spike protein prevents rapid mutational escape seen with individual antibodies. Science (New York, N.Y, doi:10.1126/science.abd0831 (2020).
17 Liu, A., Li, Y., Peng, J., Huang, Y. & Xu, D. Antibody responses against SARS-COV-2 in COVID-19 patients. Journal of medical virology, doi:10.1002/jmv.26241 (2020).
18 Long, Q. X. et al. Antibody responses to SARS-COV-2 in patients with COVID-19. Nat Med 26, 845-848, doi:10.1038/s41591-020-0897-1 (2020).
19 Wu, Y. et al. A noncompeting pair of human neutralizing antibodies block COVID-19 virus binding to its receptor ACE2. Science (New York, N.Y 368, 1274-1278, doi:10.1126/science.abc2241 (2020).
20 Brouwer, P. J. M. et al. Potent neutralizing antibodies from COVID-19 patients define multiple targets of vulnerability. Science (New York, N.Y, doi:10.1126/science.abc5902 (2020).
21 Rogers, T. F. et al. Isolation of potent SARS-COV-2 neutralizing antibodies and protection from disease in a small animal model. Science (New York, N.Y, doi:10.1126/science.abc7520 (2020).
22 Hansen, J. et al. Studies in humanized mice and convalescent humans yield a SARS-CoV-2 antibody cocktail. Science (New York, N.Y, doi:10.1126/science.abd0827 (2020).
23 Shi, R. et al. A human neutralizing antibody targets the receptor-binding site of SARS-CoV-2. Nature, doi:10.1038/s41586-020-2381-y (2020).
24 Ju, B. et al. Human neutralizing antibodies elicited by SARS-COV-2 infection. Nature, doi:10.1038/s41586-020-2380-z (2020).
25 Chen, X. et al. Human monoclonal antibodies block the binding of SARS-COV-2 spike protein to angiotensin converting enzyme 2 receptor. Cell Mol Immunol 17, 647-649, doi:10.1038/s41423-020-0426-7 (2020).
26 Monteil, V. et al. Inhibition of SARS-COV-2 Infections in Engineered Human Tissues Using Clinical-Grade Soluble Human ACE2. Cell 181, 905-913 c907, doi:10.1016/j.cell.2020.04.004 (2020).
27 Batlle, D., Wysocki, J. & Satchell, K. Soluble angiotensin-converting enzyme 2: a potential approach for coronavirus infection therapy? Clin Sci (Lond) 134, 543-545, doi:10.1042/CS20200163 (2020).
28 Davidson, A. M., Wysocki, J. & Batlle, D. Interaction of SARS-COV-2 and Other Coronavirus With ACE (Angiotensin-Converting Enzyme)-2 as Their Main Receptor: Therapeutic Implications. Hypertension 76, 1339-1349, doi:10.1161/HYPERTENSIONAHA.120.15256 (2020).
29 Wysocki, J. et al. A Novel Soluble ACE2 Variant with Prolonged Duration of Action Neutralizes SARS-COV-2 Infection in Human Kidney Organoids. J Am Soc Nephrol, doi:10.1681/ASN.2020101537 (2021).
30 Hassler, L. et al. A novel soluble ACE2 protein totally protects from lethal disease caused by SARS-COV-2 infection. bioRxiv, doi:https://doi.org/10.1101/2021.03.12.435191 (2021).
31 Weinreich, D. M. et al. REGN-COV2, a Neutralizing Antibody Cocktail, in Outpatients with Covid-19. N Engl J Med, doi:10.1056/NEJMoa2035002 (2020).
32 Group, A.-T. L.-C. S. et al. A Neutralizing Monoclonal Antibody for Hospitalized Patients with Covid-19. N Engl J Med, doi:10.1056/NEJMoa2033130 (2020).
33 Planas, D. et al. Reduced sensitivity of SARS-COV-2 variant Delta to antibody neutralization. Nature, doi:10.1038/s41586-021-03777-9 (2021).
34 Joyner MJ, R. E. C., Jonathon W. Senefeld, Stephen A. Klassen, John R. Mills, Patrick W. Johnson, Elitza S. Theel, Chad C. Wiggins, Katelyn A. Bruno, Allan M. Klompas, Elizabeth R. Lesser, Katie L. Kunze, Matthew A. Sexton, Juan C. Diaz Soto, Sarah E. Baker, John R.A. Shepherd, Noud van Helmond, Nicole C. Verdun, Peter Marks, Camille M. van Buskirk, Jeffrey L. Winters, James R. Stubbs, Robert F. Rea, David O. Hodge, Vitaly Herasevich, Emily R. Whelan, Andrew J. Clayburn, Kathryn F. Larson, Juan G. Ripoll, Kylie J. Andersen, Matthew R. Buras, Matthew N.P. Vogt, Joshua J. Dennis, Riley J. Regimbal, Philippe R. Bauer, Janis E. Blair, Nigel S. Paneth, DeLisa Fairweather, R. Scott Wright, and Arturo Casadevall. Convalescent Plasma Antibody Levels and the Risk of Death from Covid-19. NEJM, doi:10.1056/NEJMoa2031893 (2021).
35 Bloch, E. M. et al. Deployment of convalescent plasma for the prevention and treatment of COVID-19. J Clin Invest 130, 2757-2765, doi:10.1172/JCI138745 (2020).
36 Zhang, H. et al. Identification of distinct nanoparticles and subsets of extracellular vesicles by asymmetric flow field-flow fractionation. Nat Cell Biol 20, 332-343, doi:10.1038/s41556-018-0040-4 (2018).
37 Thery, C. et al. Minimal information for studies of extracellular vesicles 2018 (MISEV2018): a position statement of the International Society for Extracellular Vesicles and update of the MISEV2014 guidelines. J Extracell Vesicles 7, 1535750, doi:10.1080/20013078.2018.1535750 (2018).
38 Thery, C., Zitvogel, L. & Amigorena, S. Exosomes: composition, biogenesis and function. Nat Rev Immunol 2, 569-579, doi:10.1038/nri855 (2002).
39 Stoorvogel, W., Kleijmeer, M. J., Geuze, H. J. & Raposo, G. The biogenesis and functions of exosomes. Traffic 3, 321-330, doi:10.1034/j.1600-0854.2002.30502.x (2002).
40 Costa-Silva, B. et al. Pancreatic cancer exosomes initiate pre-metastatic niche formation in the liver. Nat Cell Biol 17, 816-826, doi:10.1038/ncb3169 (2015).
41 Peinado, H. et al. Melanoma exosomes educate bone marrow progenitor cells toward a pro-metastatic phenotype through MET. Nat Med 18, 883-891, doi:10.1038/nm.2753 (2012).
42 Kamerkar, S. et al. Exosomes facilitate therapeutic targeting of oncogenic KRAS in pancreatic cancer. Nature 546, 498-503, doi:10.1038/nature22341 (2017).
43 Kibria, G. et al. A rapid, automated surface protein profiling of single circulating exosomes in human blood. Sci Rep 6, 36502, doi:10.1038/srep36502 (2016).
44 Kibria, G., Ramos, E. K., Wan, Y., Gius, D. R. & Liu, H. Exosomes as a Drug Delivery System in Cancer Therapy: Potential and Challenges. Mol Pharm 15, 3625-3633, doi:10.1021/acs.molpharmaceut.8b00277 (2018).
45 Chen, G. et al. Exosomal PD-L1 contributes to immunosuppression and is associated with anti-PD-1 response. Nature 560, 382-386, doi:10.1038/s41586-018-0392-8 (2018).
46 Poggio, M. et al. Suppression of Exosomal PD-L1 Induces Systemic Anti-tumor Immunity and Memory. Cell 177, 414-427 e413, doi:10.1016/j.cell.2019.02.016 (2019).
47 Murphy, D. E. et al. Extracellular vesicle-based therapeutics: natural versus engineered targeting and trafficking. Exp Mol Med 51, 1-12, doi:10.1038/s12276-019-0223-5 (2019).
48 Mendt, M. et al. Generation and testing of clinical-grade exosomes for pancreatic cancer. JCI Insight 3, doi:10.1172/jci.insight.99263 (2018).
49 Chen, L., Xiang, B., Wang, X. & Xiang, C. Exosomes derived from human menstrual blood-derived stem cells alleviate fulminant hepatic failure. Stem Cell Res Ther 8, 9, doi:10.1186/s13287-016-0453-6 (2017).
50 Kugeratski, F. G. et al. Quantitative proteomics identifies the core proteome of exosomes with syntenin-1 as the highest abundant protein and a putative universal biomarker. Nat Cell Biol 23, 631-641, doi:10.1038/s41556-021-00693-y (2021).
51 Lindeman, R. M., PF; Gold, RZ. Introduction to bivariate and multivariate analysis. Vereniging voor Statistiek Bulletin 14, 11--14 (1980).
52 Groemping, U. Relative Importance for Linear Regression in R: The Package relaimpo. 2006 17, 27, doi:10.18637/jss.v017.i01 (2006).
53 Bao, L. et al. The pathogenicity of SARS-COV-2 in hACE2 transgenic mice. Nature 583, 830-833, doi:10.1038/s41586-020-2312-y (2020).
54 Jiang, R. D. et al. Pathogenesis of SARS-COV-2 in Transgenic Mice Expressing Human Angiotensin-Converting Enzyme 2. Cell 182, 50-58 c58, doi:10.1016/j.cell.2020.05.027 (2020).
55 A., L. B. Syrian hamsters as a small animal model for COVID-19 research. Lab Animal 49, 223, doi:https://doi.org/10.1038/s41684-020-0614-1 (2020).
56 Boudewijns, R. et al. STAT2 signaling restricts viral dissemination but drives severe pneumonia in SARS-COV-2 infected hamsters. Nat Commun 11, 5838, doi:10.1038/s41467-020-19684-y (2020).
57 Munoz-Fontela, C. et al. Animal models for COVID-19. Nature 586, 509-515, doi:10.1038/s41586-020-2787-6 (2020).
58 Vlassov, A. V., Magdaleno, S., Setterquist, R. & Conrad, R. Exosomes: current knowledge of their composition, biological functions, and diagnostic and therapeutic potentials. Biochimica et biophysica acta 1820, 940-948, doi:10.1016/j.bbagen.2012.03.017 (2012).
59 McAndrews, K. M., Che, S. P. Y., LeBleu, V. S. & Kalluri, R. Effective delivery of STING agonist using exosomes suppresses tumor growth and enhances anti-tumor immunity. J Biol Chem, 100523, doi:10.1016/j.jbc.2021.100523 (2021).
60 Cherian, S. et al. SARS-COV-2 Spike Mutations, L452R, T478K, E484Q and P681R, in the Second Wave of COVID-19 in Maharashtra, India. Microorganisms 9, doi:10.3390/microorganisms9071542 (2021).
61 Lci, C. et al. Neutralization of SARS-COV-2 spike pseudotyped virus by recombinant ACE2-Ig. Nat Commun 11, 2070, doi:10.1038/s41467-020-16048-4 (2020).
62 Tada, T. et al. An ACE2 Microbody Containing a Single Immunoglobulin Fc Domain Is a Potent Inhibitor of SARS-COV-2. Cell Rep 33, 108528, doi:10.1016/j.celrep.2020.108528 (2020).
63 Linsky, T. W. et al. De novo design of potent and resilient hACE2 decoys to neutralize SARS-COV-2. Science 370, 1208-1214, doi:10.1126/science.abc0075 (2020).
64 Tu, Y. F. et al. A Review of SARS-COV-2 and the Ongoing Clinical Trials. Int J Mol Sci 21, doi:10.3390/ijms21072657 (2020).
65 Ison, M. G., Wolfe, C. & Boucher, H. W. Emergency Use Authorization of Remdesivir: The Need for a Transparent Distribution Process. JAMA 323, 2365-2366, doi:10.1001/jama.2020.8863 (2020).
66 Roefs, M. T., Sluijter, J. P. G. & Vader, P. Extracellular Vesicle-Associated Proteins in Tissue Repair. Trends Cell Biol, doi:10.1016/j.tcb.2020.09.009 (2020).
67 Walsh, K. A. et al. SARS-COV-2 detection, viral load and infectivity over the course of an infection. J Infect 81, 357-371, doi:10.1016/j.jinf.2020.06.067 (2020).
68 Jeppesen, D. K. et al. Reassessment of Exosome Composition. Cell 177, 428-445 c418, doi:10.1016/j.cell.2019.02.029 (2019).
69 Zivanov, J. et al. New tools for automated high-resolution cryo-EM structure determination in RELION-3. Elife 7, doi:10.7554/eLife.42166 (2018).
70 Schneider, C. A., Rasband, W. S. & Eliceiri, K. W. NIH Image to ImageJ: 25 years of image analysis. Nat Methods 9, 671-675, doi:10.1038/nmeth.2089 (2012).
71 Amanat, F. et al. A serological assay to detect SARS-COV-2 seroconversion in humans. medRxiv: the preprint server for health sciences, doi:10.1101/2020.03.17.20037713(2020).
72 McDade, T. W. et al. High seroprevalence for SARS-COV-2 among household members of essential workers detected using a dried blood spot assay. PloS one 15, c0237833, doi:10.1371/journal.pone.0237833 (2020).
73 Yuan, M. et al. A highly conserved cryptic epitope in the receptor binding domains of SARS-COV-2 and SARS-COV. Science (New York, N.Y.) 368, 630-633, doi:10.1126/science.abb7269 (2020).
74 Tsai, C. F. et al. An Improved Boosting to Amplify Signal with Isobaric Labeling (iBASIL) Strategy for Precise Quantitative Single-cell Proteomics. Mol Cell Proteomics 19, 828-838, doi:10.1074/mcp.RA119.001857 (2020).
75 Kong, A. T., Leprevost, F. V., Avtonomov, D. M., Mellacheruvu, D. & Nesvizhskii, A. I. MSFragger: ultrafast and comprehensive peptide identification in mass spectrometry-based proteomics. Nat Methods 14, 513-520, doi:10.1038/nmeth.4256 (2017).
76 Teo, G. C., Polasky, D. A., Yu, F. & Nesvizhskii, A. I. Fast Deisotoping Algorithm and Its Implementation in the MSFragger Search Engine. J Proteome Res 20, 498-505, doi:10.1021/acs.jproteome.0c00544 (2021).
77 Okuda, S. et al. jPOSTrepo: an international standard data repository for protcomes. Nucleic Acids Res 45, D1107-D1111, doi:10.1093/nar/gkw 1080 (2017).
78 Vizcaino, J. A. et al. ProtcomeXchange provides globally coordinated proteomics data submission and dissemination. Nat Biotechnol 32, 223-226, doi:10.1038/nbt.2839 (2014).
79 Zheng, J. et al. COVID-19 treatments and pathogenesis including anosmia in K18-hACE2 mice. Nature 589, 603-607, doi:10.1038/s41586-020-2943-z (2021).

### Example 2: Detection of ACE2⁺ Exosome and Human IgG (H+L)⁺ Exosomes in the Plasma

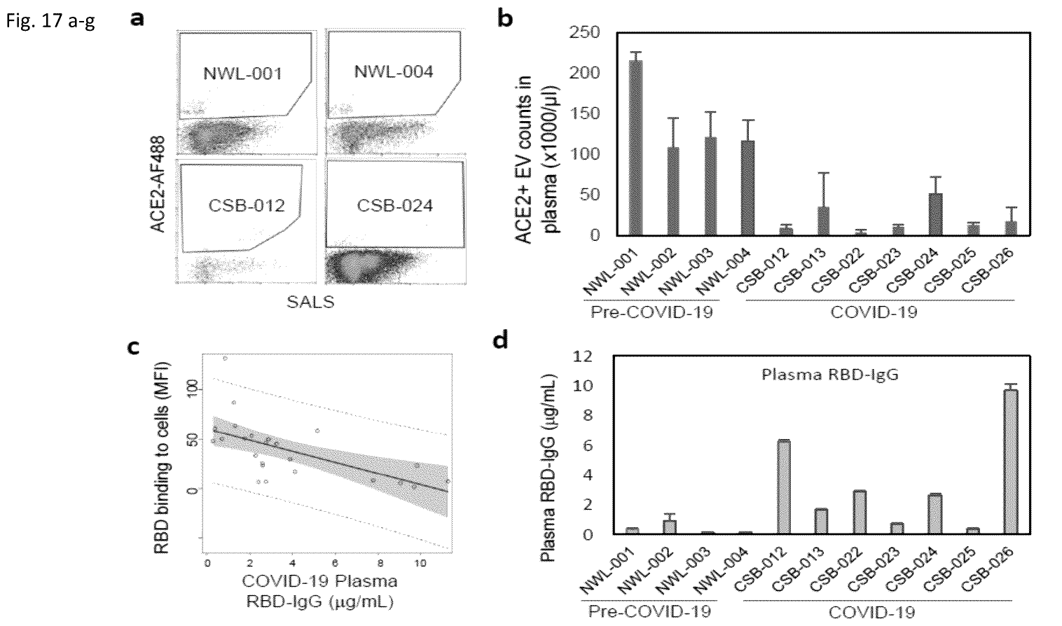

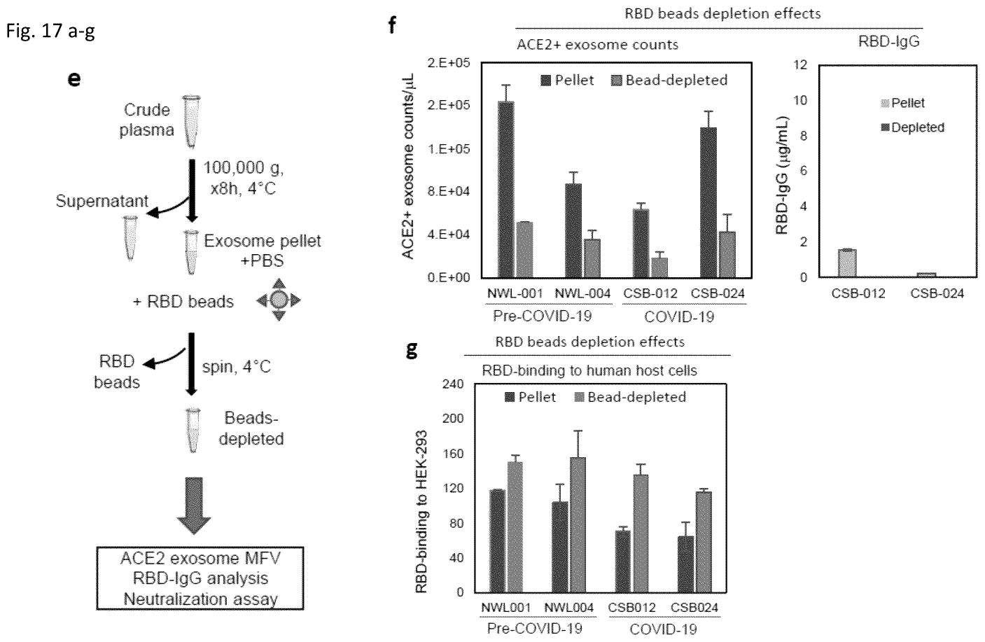

We have detected ACE2+ exosomes in the plasma of both pre-COVID-19 patients and COVID-19 convalescent patients (see Example 1), In addition, we have detected RBD-IgG in the plasma of COVID-19 convalescent patients. The RBD-IgG and Spike-specific IgG levels arc exclusively detected in COVID-19 plasma whereas the pre-COVID-19 plasma are negative of such viral antigen-specific antibodies. The RBD-IgG is positively associated with the neutralization capacity to inhibit RBD binding to ACE2+ human host cells (HEK-293). Depletion of RBD-bound exosomes (ACE2+ in pre-COVID-19) via RBD-beads restored RBD binding to host cells (FIG. 17e-g).

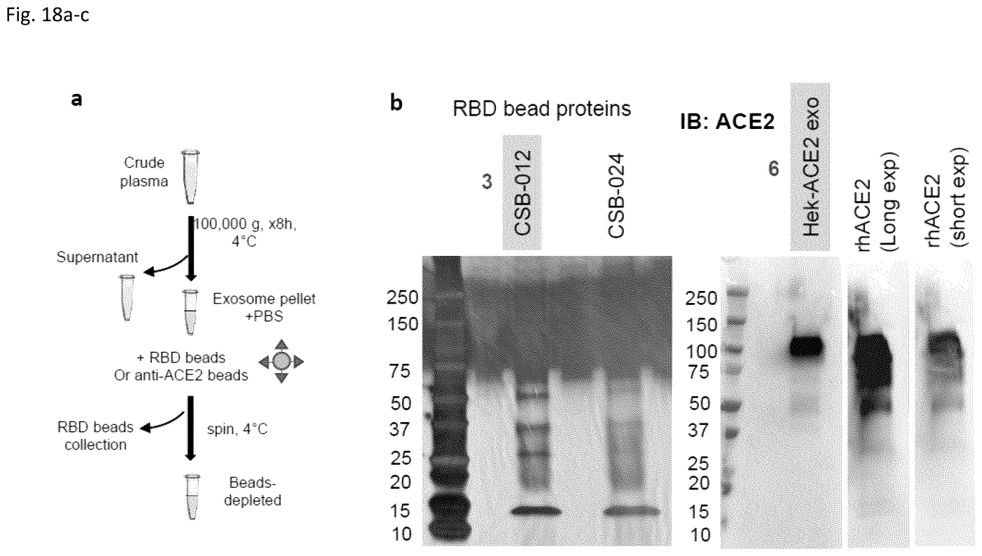

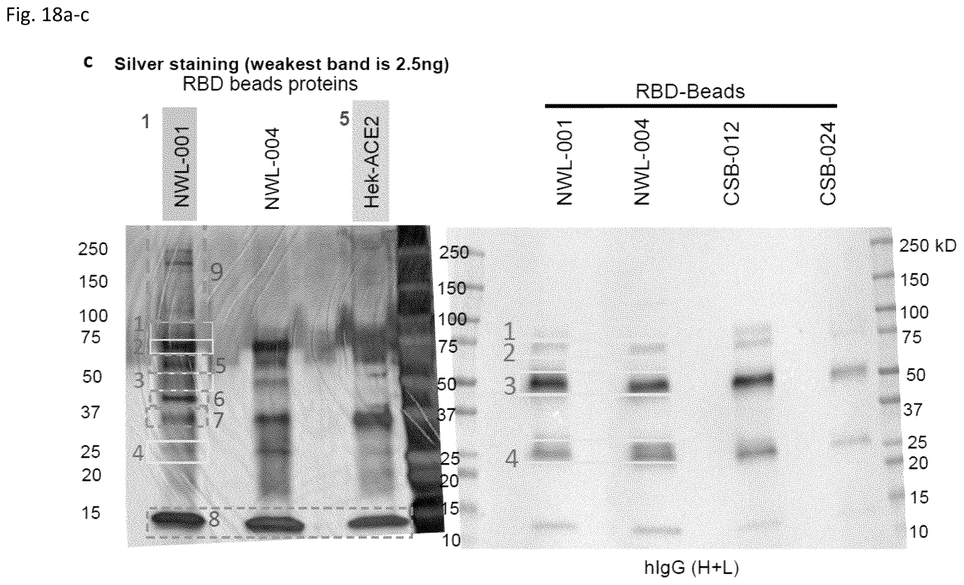

Intriguingly, depletion of exosomes from the COVID-19 convalescent plasma by overnight ultracentrifugation (18 h) completely abolished the RBD neutralization capacity in host cell binding (FIG. 15a-b). In the COVID-19 plasma, the ACE2+ exosomes are relatively low whereas RBD-IgG levels are positively associated with RBD neutralization (FIG. 17c-d). The RBD-IgG is depleted from plasma upon ultracentrifugation (100,000 g) in a time-dependent manner and subsequently enriched in the pellet (FIG. 15c-d). The immunoblotting of RBD-bead proteins after 8-hour ultracentrifugation show human IgG is enriched in the pellet, which is also confirmed by single exosome analysis on Apogee MFV. Ongoing studies will characterize the ACE2 and hIgG in RBD-bound exosomes using proteomic mass spec (FIG. 18. sliver staining gel).

Engineering of RBD-binding IgG clones for expression in HEK-293 cells.

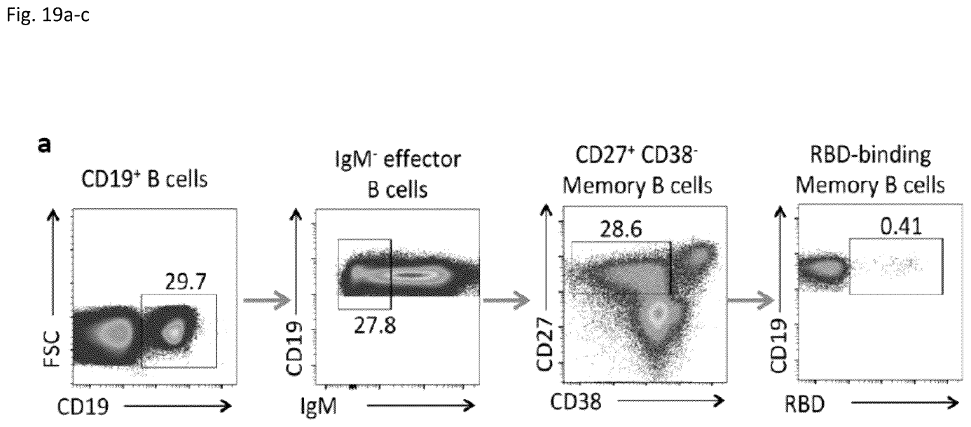

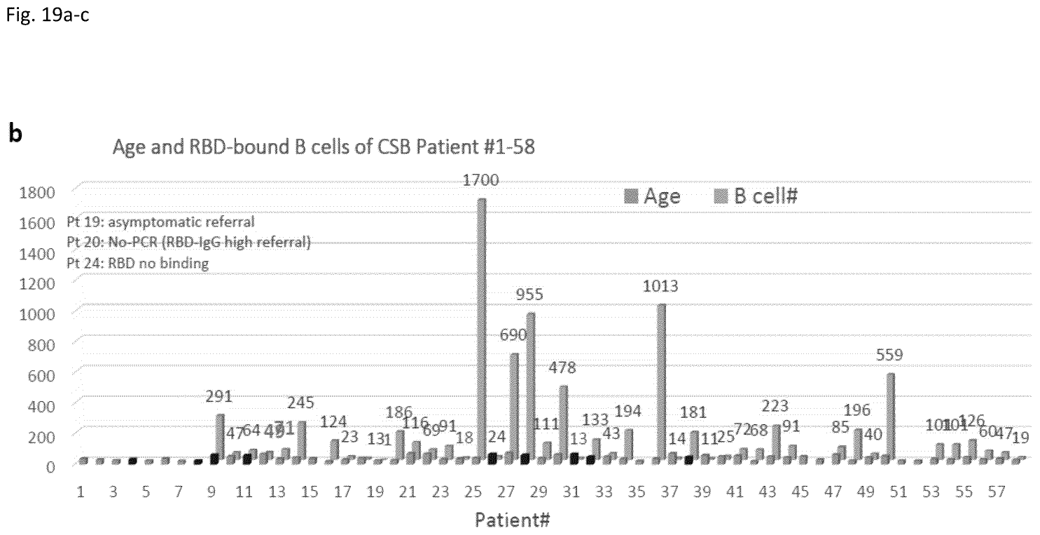

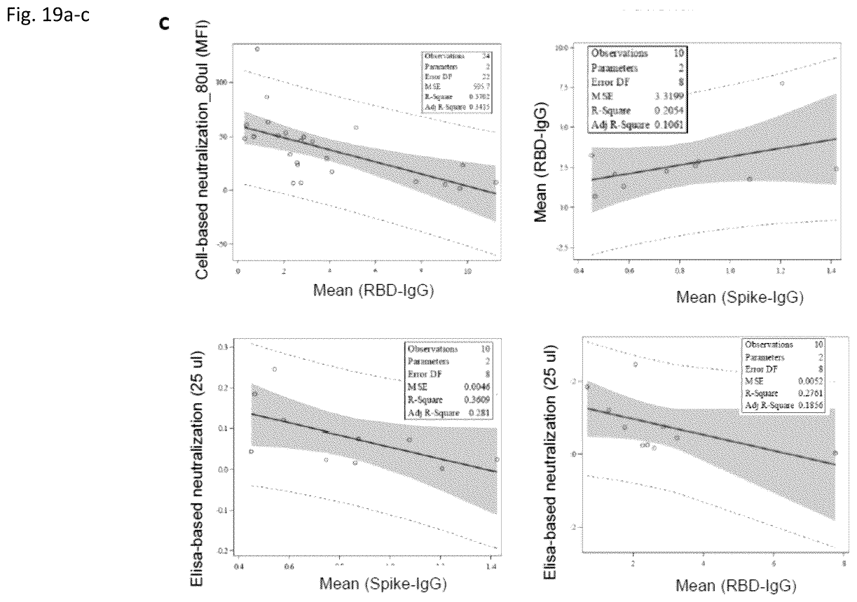

We have sorted ˜7,000 RBD-specific B cells (IgM-CD38-CD27^{med}RBD⁺memory B) from 50 COVID-19 convalescent patients for single-cell RNA seq using 10×genomic reagents (FIG. 19a-b). The plasma RBD-IgG is positively associated with neutralization function to block RBD binding to human host cells (ACE2+ HEK cells), spike-specific IgG levels, and ELISA-based RBD-ACE2 interactions (FIG. 19c).

The AbVec vectors with constant regions of IgG heavy chains (mainly IgG1, and possibly IgG2, IgG3, and IgG4) and L chains (kappa/K and lambda/L) are used for cloning with inserted variable regions of H and L chain sequences obtained from B cell sequencing. In order to engineer IgG+exosomes for neutralization functional evaluation, the coding sequence of the IgG1transmembrane domain and cytoplasmic tail of 69 amino acids are added to the C-terminus of the soluble IgG sequence in the existing AbVecIgG1 vector.

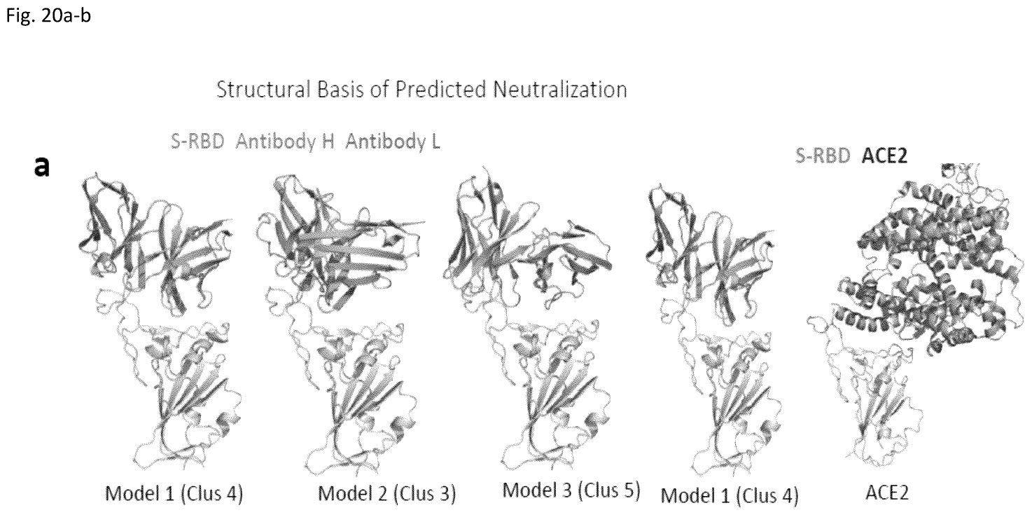

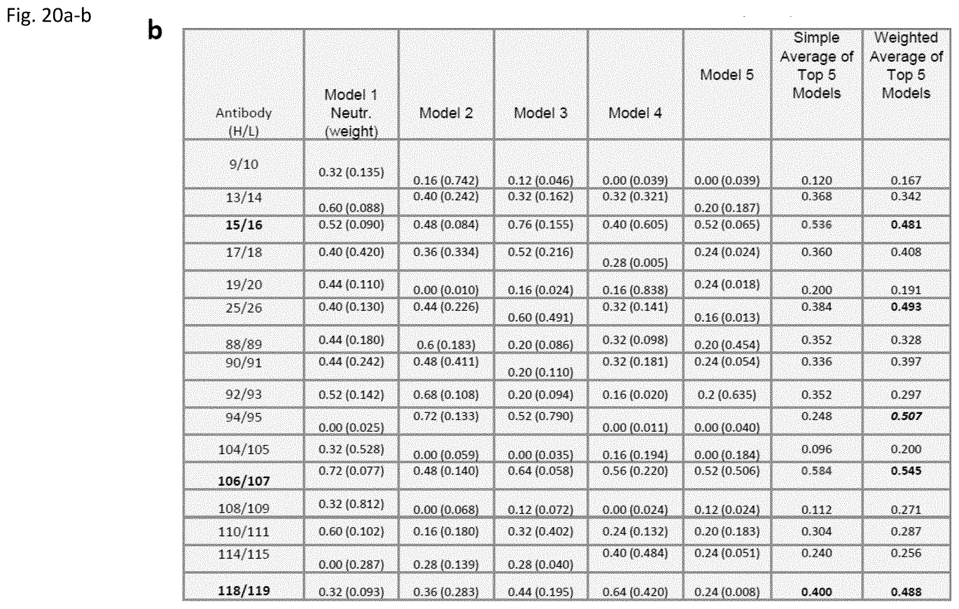

Structure and machine learning-based prediction and optimization of RBD-specific IgG clones (FIG. 20).

### REFERENCES

1. U.S. patent application Ser. No. 15/788,709
2. Kamerkar S, LeBleu VS, Sugimoto H, Yang S, Ruivo CF, Melo SA, Lee JJ, Kalluri R. Exosomes facilitate therapeutic targeting of oncogenic KRAS in pancreatic cancer. Nature. 2017; 546 (7659): 498-503. doi:10.1038/nature22341. PubMed PMID: 28607485; PMCID: PMC5538883.
3. Monteil V, Kwon H, Prado P, Hagelkruys A, Wimmer RA, Stahl M, Leopoldi A, Garreta E, Hurtado Del Pozo C, Prosper F, Romero JP, Wirnsberger G, Zhang H, Slutsky AS, Conder R, Montserrat N, Mirazimi A, Penninger JM. Inhibition of SARS-COV-2 Infections in Engineered Human Tissues Using Clinical-Grade Soluble Human ACE2. Cell. 2020. doi: 10.1016/j.cell.2020.04.004. PubMed PMID: 32333836; PMCID: PMC7181998.
4. A human neutralizing antibody targets the receptor binding site of SARS-COV-2 https://world wide web, nature dot com front slash articles front slash s41586-020-2381-y.
5. Potent neutralizing antibodies against SARS-COV-2 identified by high-throughput single-cell sequencing of convalescent patients' B cells at https://world wide web dot science direct dot com front slash science front slash article front slash pii/S0092867420306206.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1-20. (canceled)

21. An engineered exosome comprising one or more of ACE2 protein, or SARS-COV-2 specific IgG (IgG), or Coronavirus Spike, or a fragment or variant thereof.

22. The engineered exosome of claim 21, wherein the exosome comprises the IgG or the fragment or variant thereof.

23. The engineered exosome of claim 22, wherein the IgG or the fragment or variant thereof represents at least about 10%, 20%, 30%, 40%, 50%, 60%, 70%, 80%, 90% or more of the total protein comprised by the engineered exosome.

24. The engineered exosome of claim 22, wherein the IgG or the fragment or variant thereof binds to the spike protein of a coronavirus selected from SARS-COV-2, SARS-COV, and HCoV-NL63.

25. The engineered exosome of claim 22, wherein the IgG or the fragment or variant thereof comprises a heavy chain variable region encoded by a polynucleotide having at least 80% identity to SEQ ID NO: 45.

26. The engineered exosome of claim 22, wherein the IgG or the fragment or variant thereof comprises a light chain variable region encoded by a polynucleotide having at least 80% identity to SEQ ID NO: 46.

27. The engineered exosome of claim 22, wherein the IgG or the fragment or variant thereof comprises: a heavy chain variable region encoded by a polynucleotide having at least 80% identity to SEQ ID NO: 45; and a light chain variable region encoded by a polynucleotide having at least 80% identity to SEQ ID NO: 46.

28. The engineered exosome of claim 27, wherein the IgG or the fragment or variant thereof comprises a transmembrane domain and/or a cytoplasmic tail.

29. The engineered exosome of claim 28, wherein the transmembrane domain is an IgG1 transmembrane domain.

30. The engineered exosome of claim 22, wherein the exosome comprises an additional active agent for treating and/or preventing infection by a coronavirus selected from SARS-COV-2, SARS-COV, and HCoV-NL63.

31. The engineered exosome of claim 30, wherein the additional active agent comprises remedsivir.

32. The engineered exosome of claim 21, wherein the exosome has an effective average diameter in the range of about 30-150 nm.

33. A pharmaceutical composition comprising the engineered exosome of claim 22.

34. The pharmaceutical composition of claim 33, wherein the composition is formulated as an aerosol, a lyophilized powder, or an emulsion.

35. A system for delivering the aerosol formulated pharmaceutical composition of claim 34, comprising a device for administering the aerosol (e.g. an inhaler or a nebulizer).

36. A method of treating a subject diagnosed with a viral infection, the method comprising administering to the subject an effective amount of the engineered exosomes of claim 22.

37. The method of claim 36, wherein the virus is SARS-COV or HCoV-NL63.


SEQUENCE LISTING (72 sequences)

SEQ ID NO: 1 - protein, 805 aa, Homo sapiens [sequence omitted]

SEQ ID NO: 2 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 539 aa, synthetic construct
  REGION 1..539: note = Synthetic- Ig heavy chain protein sequence hIgG1-0001 (1-539) with TMD
MGWSCIILFLVATATGVHSEVQLVQSGAEVKKPGASVKVSCKASGYTFTDYYIHWVRQAPGQGLEWMGWINPISGGTNYAQKFQGRVTMTRDTSVTTFYMELSWLTSDDSAVYYCARCPFFYSETSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPQLEESCAEAQDGELDGLWTTITIFITLFLLSVCYSATVTFFKVKWIFSSVVDLKQTIIPDYRNMIGQGA

SEQ ID NO: 4 - protein, 472 aa, synthetic construct
  REGION 1..472: note = Synthetic- Ig heavy chain protein sequence hIgG1-0001 (1-472) without TMD
MGWSCIILFLVATATGVHSEVQLVQSGAEVKKPGASVKVSCKASGYTFTDYYIHWVRQAPGQGLEWMGWINPISGGTNYAQKFQGRVTMTRDTSVTTFYMELSWLTSDDSAVYYCARCPFFYSETSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 235 aa, synthetic construct
  REGION 1..235: note = Synthetic- Ig light chain protein sequence hIgL-0002 (1-235)
MGWSCIILFLVATATGSWAQSALTQPASVSGSPGQSITISCTGTSSDVGGYNYVSWFQQHPGKAPKLMIYEVSTRPSGVSNRFSGSKSGNTASLTISGLQAEDEADYYCSSYTSSNTYVFGTGTQVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - DNA, 436 bp, synthetic construct
  misc_feature 1..436: note = Synthetic- 0001-IGG1
  source 1..436: mol_type = other DNA
atcctttttctagtagcaactgcaaccggtgtacattccgaggtgcagctggtgcagtctggggctgaggtgaagaagcctggggcctcagtgaaggtctcctgcaaggcttctggatacaccttcaccgactactatatacactgggtgcgacaggcccctggacaagggcttgagtggatgggatggatcaaccctataagtggtggcacaaactatgcacagaagtttcagggcagggtcaccatgaccagggacacgtccgtcaccactttttacatggagctgagctggctgacatctgacgactcggccgtatattactgtgcgagatgcccgttcttttactctgaaactagtggttatttcgactactggggccagggaaccctggtcaccgtctcctcagcgtcgaccaagggcccatcggtcttcc

SEQ ID NO: 7 - DNA, 436 bp, synthetic construct
  misc_feature 1..436: note = Synthetic- 0002-IGL1
  source 1..436: mol_type = other DNA
atcctttttctagtagcaactgcaaccggttcctgggcccagtctgccctgactcagcctgcctccgtgtctgggtctcctggacagtcgattaccatctcctgcactggaaccagcagtgacgttggtggttataactatgtctcctggttccaacagcacccaggcaaagcccccaaactcatgatttatgaggtcagtactcggccctcaggggtttctaatcgcttctctggctccaagtctggcaacacggcctccctgaccatctctgggctccaggctgaggacgaggctgattattactgcagctcatatacaagcagcaacacttatgtcttcggaactgggacccaggtcaccgtcctaggtcagcccaaggccaaccccactgtcactctgttcccaccctcgagtgaggagcttcaagccaaca

SEQ ID NO: 8 - DNA, 448 bp, synthetic construct
  misc_feature 1..448: note = Synthetic- 0007-IGG1
  source 1..448: mol_type = other DNA
atcctttttctagtagcaactgcaaccggtgtacattctgaggtgcagctggtggagtcggggggaggcttggtccagcctggggggtccctgagactctcctgtgcagcctctggattcaccttcagtagctacgacatgcactgggtccgccaagctacaggaaaaggtctggagtgggtctccactattggtactgctggtgacacatattatttaggttccgtgaagggccgatttaccttctccagagaaaatgccaagaactccttgtatcttcaaatgaacagcctgagagccggggacacggctgtgtattactgtgcaagagcgaagtactatgatagtagtggttattatcactacacgccctactactttgactattggggccagggaaccctggtcaccgtctcctcagcgtcgaccaagggcccatcggtcttcc

SEQ ID NO: 9 - DNA, 389 bp, synthetic construct
  misc_feature 1..389: note = Synthetic- 0008-IGK
  source 1..389: mol_type = other DNA
atcctttttctagtagcaactgcaaccggtgtacattctgacatccagatgacccagtctccatcctccctgtctgcatctgtaggagacagagtcaccatcacttgccgggcgagtctgggcattggaaattctttagcctggtatcagcagaaaccagggaaagcccctaaggtcctgctctatgctgcatccagattggaaagtggggtcccatccaggttcagtggcagtggatctgggacggattacactctcaccattagcagcctgcagcctgaagattttgcaacttattactgtcaacagtattacagtacccctgaggtcactttcggcggagggaccaaggtggaaatcaaacgtacggtggctgcaccatctgtctt

SEQ ID NO: 10 - DNA, 427 bp, synthetic construct
  misc_feature 1..427: note = Synthetic- 0011-IGG1
  source 1..427: mol_type = other DNA
atcctttttctagtagcaactgcaaccggtgtacattctgaggtgcagctggtggagtctgggggaggcgtggtccagcctgggaggtccctgagactctcctgtgcagcctctggattcaccttcagtcgctacggcatgcactgggtccgccaggctccaggcaaggggctggagtgggtggcagttatatcatttgatggaaggaataaatattatgcagactccgtgaagggccgattcaccatctccagagacaattccaaaaccacgctgtatttgcaaatgaacagcctgagagctgaggacacggctgtgtatcactgtgcgaaagatgggttagcagtgtcggactaccttgactactggggccagggaaccctggtcaccgtctcctcagcgtcgaccaagggcccatcggtcttcc

SEQ ID NO: 11 - DNA, 386 bp, synthetic construct
  misc_feature 1..386: note = Synthetic- 0012-IGK
  source 1..386: mol_type = other DNA
atcctttttctagtagcaactgcaaccggtgtacattctgacatccagatgacccagtctccatcctccctgtctgcatctgtaggagacagagtcaccatcacttgccgggcaagtcagagcattaccaactatttaaattggtatcagcagaaaccagggaaagcccctgagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctggcacttttggccaggggaccaaggtggaaatcaaacgtacggtggctgcaccatctgtctt

SEQ ID NO: 12 - DNA, 451 bp, synthetic construct
  misc_feature 1..451: note = Synthetic- 0013-IGG1
  source 1..451: mol_type = other DNA
atcctttttctagtagcaactgcaaccggtgtacattctgaagtgcagctggtggagtctgggggaggcgtggcccaacctgggaggtccctgagactctcctgtgcagcctctggattcaccttcagtaactatgccatgcactgggtccgccaggctccaggcaaggggctggagtgggtggcagttatatcatttgatggaagtgataaatactatgtagactccgtgaagggccgattcaccatctccagagacaattccaagaacacactgtatctgcaaatgaacagcctgagagctgaggacacggctgtgtattactgtgcgaaaagcggggggttatattgtaatggtggtaactgctactacggctactactttgactactggggccagggaaccctggtcaccgtctcctcagcgtcgaccaagggcccatcggtcttcc

SEQ ID NO: 13 - DNA, 389 bp, synthetic construct
  misc_feature 1..389: note = Synthetic- 0014-IGK
  source 1..389: mol_type = other DNA
atcctttttctagtagcaactgcaaccggtgtacattctgacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgaggcatctagtttagaaagtggggtcccatcaaggttcagcggcagtggatctgggacagaattcactctcaccatcagcagcctgcagcctgatgattttgcaacttattactgccaacagtataatacttattctccgtacacttttggccaggggaccaaggtggaaatcaaacgtacggtggctgcaccatctgtctt

SEQ ID NO: 14 - DNA, 445 bp, synthetic construct
  misc_feature 1..445: note = Synthetic- 0015-IGG1
  source 1..445: mol_type = other DNA
atcctttttctagtagcaactgcaaccggtgtacattctgaagtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatatgcaaatgaacaccctgagagctgaggacacggctgtgtattactgtgcgaaagttgtaggagcatattgtggtggtgactgccttacgggatactttgactactggggccagggaaccctggtcaccgtctcctcagcgtcgaccaagggcccatcggtcttcc

SEQ ID NO: 15 - DNA, 386 bp, synthetic construct
  misc_feature 1..386: note = Synthetic- 0016-IGK
  source 1..386: mol_type = other DNA
atcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtgcatctgggacagattttactttcaccatcagcagcctgcagcctgaagatattgcaacatattactgtcaacagtatgataatctccctctcactttcggcggagggaccaaggtggaaatcaaacgtacggtggctgcaccatctgtctt

SEQ ID NO: 16 - DNA, 436 bp, synthetic construct
  misc_feature 1..436: note = Synthetic- 0017-IGG1
  source 1..436: mol_type = other DNA
atcctttttctagtagcaactgcaaccggtgtacattctgaagtgcagctggtggagtctgggggaggcgtggtccagcctgggaggtccctgagactctcctgtgcagcctctggattcaccttcagtagccatgttatgcactgggtccgccagactccaggcaaggggctggagtgggtggcggttatatcatatgatggaagcagtaaatactacgcagactccgtgaagggccgattcaccatctccagagacaatgccaagaacacgctgtatctgcaaatgaacagcctgaaaactgaggacacggctgtgtattactgtgcgagagagcgagtaagcagtggctggtatcttgatccttttgatatctggggccaagggacaatggtcaccgtctcttcagcgtcgaccaagggcccatcggtcttcc

SEQ ID NO: 17 - DNA, 389 bp, synthetic construct
  misc_feature 1..389: note = Synthetic- 0018-IGK
  source 1..389: mol_type = other DNA
atcctttttctagtagcaactgcaaccggtgtacattctgacatccagatgacccagtctccatcctccctgtctgcatctgtaggagacagagtcaccatcacttgtcgggcaagtcagagcattagcaactatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacactaccctctcgatcaccttcggccaagggacacgactggagattaaacgtacggtggctgcaccatctgtctt

SEQ ID NO: 18 - DNA, 457 bp, synthetic construct
  misc_feature 1..457: note = Synthetic- 0019-IGG1
  source 1..457: mol_type = other DNA
atcctttttctagtagcaactgcaaccggtgtacattctgaagtgcagctggtggagtctgggggaggcgtggtccagcctgggaggtccctgagactctcctgtgcagcctctggattcaccttcagtaactatgctatgcactgggtccgccaggctccaggcaaggggctggagtgggtggcagttatatcatatgatggaagcaataaatactatgtagactccgtgaagggccgattcaccatctccagagacaattccaagagcacgctgtatctgcaaattaacagcctgagagctgaggacacggctgtctattactgtgcgagagatcgcaaaccaagttacgattcttggagtggttatacccactaccactacggtatggacgtctggggccaagggaccacggtcaccgtctcctcagcgtcgaccaagggcccatcggtcttcc

SEQ ID NO: 19 - DNA, 436 bp, synthetic construct
  misc_feature 1..436: note = Synthetic- 0020-IGL2
  source 1..436: mol_type = other DNA
atcctttttctagtagcaactgcaaccggttcctgggcccagtctgccctgactcagcctgcctccgtgtctgggtctcgtagacagtcgatcaccatctcctgcactggaaccagcagtgatgttgggagttataaccttgtctcctggttccaacatcacccaggcaaagcccccaacctcgtgatttatgaggacaataagcggccctcaggagtttctaatcgcttctctggctccaagtctggccacacggcctccctgacaatctctgggctccaggctgaggacgaggctgattattactgctgctcatatgcaggtagtggcacttgggtgttcggcggagggaccaagctgaccgtcctaagtcagcccaaggctgccccctcggtcactctgttcccaccctcgagtgaggagcttcaagccaaca

SEQ ID NO: 20 - DNA, 436 bp, synthetic construct
  misc_feature 1..436: note = Synthetic- 0025-IGG1
  source 1..436: mol_type = other DNA
atcctttttctagtagcaactgcaaccggtgtacattcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatcttttacagtgggagcaccaactacaacccctccctcaggagtcgagtcaccatatcagtggacacgcccaagaaccagttctccctgaggctgaggtctgtgaccgctgcggacacggccgtgtattactgtgcgagagactctatggatacaactacgtgggcccctacggcgtttgactactggggccagggaaccctggtcaccgtctcctcagcgtcgaccaagggcccatcggtcttcc

SEQ ID NO: 21 - DNA, 386 bp, synthetic construct
  misc_feature 1..386: note = Synthetic- 0026-IGK
  source 1..386: mol_type = other DNA
atcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcaatggaagtggatctgggacagattttactttcaccatcagcagcctgcagcctgaagatattgcaacatattactgtcaacagtatgataatctccccctcactttcggcggagggaccaaggtggaaatcaaacgtacggtggctgcaccatctgtctt

SEQ ID NO: 22 - DNA, 430 bp, synthetic construct
  misc_feature 1..430: note = Synthetic- 0031-IGG1
  source 1..430: mol_type = other DNA
atcctttttctagtagcaactgcaaccggtgtacattccgaggtgcagctggtgcagtctggggctgaggtgaagaagcctggggcctcagtgaaggtctcctgcaaggcttctggatacttcttcaccggcttctacatacactgggtgcgacaggcccctggacaagggcttgagtggatgggatggatcagccctatcagtggtggcgcaaactctgcacagacgtttcaggacagggtcaccatgaccagggacacgtccatcaccacagcctacatggagctgagcaggctgagatctgacgacacggccgtatactactgtgcgagagccccctactatgatagcagtgcttctcttgactactggggccagggaaccctggtcaccgtctcctcagcgtcgaccaagggcccatcggtcttcc

SEQ ID NO: 23 - DNA, 392 bp, synthetic construct
  misc_feature 1..392: note = Synthetic- 0032-IGK
  source 1..392: mol_type = other DNA
atcctttttctagtagcaactgcaaccggtgtacattctgacatccagatgacccagtctccatcctccctgtctgcatctgtaggagacagagtcaccatcacttgccaggcgagtcaggacattagcaactctttaaattggtatcaccagaaaccagggaaagcccctaggctcctgatctacgatgcatccaatttgaaaacaggggtcccatcaaggttcagtggaagtggatctgggacagattttactttcaccatcagcagcctgcagcctgaagatattggaacattttactgtcaacagtatgataatctccctcctgccctcactttcggccctgggaccaaagtggatatcaaacgtacggtggctgcaccatctgtctt

SEQ ID NO: 24 - DNA, 445 bp, synthetic construct
  misc_feature 1..445: note = Synthetic- 0039-IGG1
  source 1..445: mol_type = other DNA
atcctttttctagtagcaactgcaaccggtgtacattctgaggtgcagctggtggagtctgggggaggcttggtacagcctggggggtccctgagactctcctgtgcagcctctggattcaccttcagtaggtacgacatgcactgggtccgccaagctacaggaaaaggtctggagtgggtctcagctattggtacttctggtgacacatactatccaggctccgtgaggggccgattcaccatctccagagaaaatgccaagaactccttgtatcttcaaatgaacagcctgagagccggggacacggctgtgtattactgtgcaagagtcaactatgatagtggtggttacggaatacgggaatactggttcttcgatctctggggccgtggcaccctggtcaccgtctcctcagcgtcgaccaagggcccatcggtcttcc

SEQ ID NO: 25 - DNA, 389 bp, synthetic construct
  misc_feature 1..389: note = Synthetic- 0040-IGK
  source 1..389: mol_type = other DNA
atcctttttctagtagcaactgcaaccggtgtacattctgacatccagatgacccagtctccatcctccctgtctgcatctgtaggagacagagtcaccatcacttgccgggcaagtcagagcattagcagctttttaaattggtatcagcagaaaccagggaaagcccctaaactcctaatctatgctgcatccagtttgcagagtggggtcccatcaaggttcagtggcagtggatctgggacagatttcactctcaccatcagcagtctccaacctgaagattttgcaacttacttctgtcaacagagttacagtacccctccgtggacgttcggccaggggaccaaggtggaaatcaaacgtacggtggctgcaccatctgtctt

SEQ ID NO: 26 - DNA, 457 bp, synthetic construct
  misc_feature 1..457: note = Synthetic- 0043-IGG1
  source 1..457: mol_type = other DNA
atcctttttctagtagcaactgcaaccggtgtacattctgaggtgcagctgttggagtcggggggaggcttggtgcagcctggggggtccctgagactctcctgctcagcttctggattcacctttggcacctatgccatgagctgggtccgccaggctccggggaaggggctggagtgcgtctcaactattgatgatatttatggtagtggtggtaggaccttctacgcaggctccgtgcacggccgcttcaccatttcgagagacaattccaagaacacgctgtatctgcagatgaacagcctgagagccgaggacacggccatatattactgtgcgagagataaatatcactatgatagtggtggttattatcgcctggcgggacttgactactggggccagggaaccctggtcaccgtctcctcagcgtcgaccaagggcccatcggtcttcc

SEQ ID NO: 27 - DNA, 386 bp, synthetic construct
  misc_feature 1..386: note = Synthetic- 0044-IGK
  source 1..386: mol_type = other DNA
atcctttttctagtagcaactgcaaccggtgtacattcagacatccagttgacccagtctccatcctccctgtctgcatctgtaggagacagagtcaccatcacttgccgggccagtcagggcattagcagttatttagcctggtatcagcaaaaaccagggaaagcccctaagctcctgatctttggtgcatccactttgcaaagtggggccccatcaaggttccgcggcagtggatctgggacagatttcactctcgccatcagcaacctgcagcctgaagattttgcaacttattactgtcaacagactgatagttaccctcggacgttcggccaagggaccaaggtggaaatcaaacgtacggtggctgcaccatctgtctt

SEQ ID NO: 28 - DNA, 445 bp, synthetic construct
  misc_feature 1..445: note = Synthetic- 0051-IGG1
  source 1..445: mol_type = other DNA
atcctttttctagtagcaactgcaaccggtgtacattctgaagtgcagctggtggagtctgggggaggcgtggtccagcctgggaggtccctgagactctcctgtgcagcctctggattcaccttcagtagctatggcatgcactgggtccgccaggctccaggcaaggggctggagtgggtggctagtatatcatatgatggaagtgaatattatgcagagtccgtgaagggccgattcaccatctccagagacaattccaagagcacgctgcatctgcaaatgaaaagcctgagagctgaggacacggctgtgtattactgtgcgaaaaatggggggccctattgtagtggtggtggctgctacggatcgtactttgactactggggccagggaaccctggtcaccgtctcctcagcgtcgaccaagggcccatcggtcttcc

SEQ ID NO: 29 - DNA, 386 bp, synthetic construct
  misc_feature 1..386: note = Synthetic- 0052-IGK
  source 1..386: mol_type = other DNA
atcctttttctagtagcaactgcaaccggtgtacattctgacatccagatgacccagtctccaccctccctgtctgcctctgtaggagacagagtcaccatcacttgccgggcaagtcagagcattagcagctatttaaattggtatcagcagaaaccagggaacgcccctaagctcctgatctttgctgcatccagtttggaaactggggtcccatcaaggttcagtggcagtggatctgggacagatttcactctcaccatcaacagtctgcaacctgaagattttgcaacttactactgccaacagagttccagtgcccccttaactttcggccctgggaccaaagtggatatcaaacgtacggtggctgcaccatctgtctt

SEQ ID NO: 30 - DNA, 395 bp, synthetic construct
  misc_feature 1..395: note = Synthetic- 0053-IGK
  source 1..395: mol_type = other DNA
atcctttttctagtagcaactgcaaccggtgtacatggggatattgtgatgacccagactccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactcatttcacactgaaaatcagcagggtggaggctgaggatgtttggctttattactgcatgcaaggtacacactggctcttcggcggagggaccaaggtggaaatcaaacgtacggtggctgcaccatctgtctt

SEQ ID NO: 31 - DNA, 421 bp, synthetic construct
  misc_feature 1..421: note = Synthetic- 0086-IGG1
  source 1..421: mol_type = other DNA
atcctttttctagtagcaactgcaaccggtgtacattccgaggtgcagctggtgcagtctggagcagaggtgaaaaagcccggggagtctctgaagatctcctgtaagggttctggatacagctttattagcaactggatcggctgggtgcgccagatgcccgggaaaggcctggagtggatggggagcatctatcctggtgactctgacaccagatacagtccgtccttccaaggccaggtcaccatctcagccgacaagtccatcagcaccgcctacctgcagtggagcagcctgaaggcctcggacaccgccatatattactgtgcgagactggagtcagactggtacttcgatctctggggccgtggcaccctggtcaccgtctcctcagcgtcgaccaagggcccatcggtcttcc

SEQ ID NO: 32 - DNA, 427 bp, synthetic construct
  misc_feature 1..427: note = Synthetic- 0087-IGL2
  source 1..427: mol_type = other DNA
atcctttttctagtagcaactgcaaccggttctgtgacctcctatgagctgacwcaggaccctgctgtgtctgtggccttgggacagacagtcaggatcacatgccaaggagacagcctcagaagccattatgcaagctggtaccagcagaagccaggacaggcccctgtagttgtcatctatggtaaagacaaccggccctcagggatcccagaccgattctctggctccagctcaggaaatacagcttccttgaccatcactggggctcaggcggaagatgaggctgactactactgtaactcccgggacagcagtggaaaccatcctttcggcggagggaccaagctgaccgtcctaggtcagcccaaggctgccccctcggtcactctgttcccaccctcgagtgaggagcttcaagccaaca

SEQ ID NO: 33 - DNA, 418 bp, synthetic construct
  misc_feature 1..418: note = Synthetic- 0088-IGG1
  source 1..418: mol_type = other DNA
atcctttttctagtagcaactgcaaccggtgtacattcccaggtgcagctgcaggagtctgggggaggcttggtccagcctggggggtccctgagactctcctgtgcagcctctggattcaccgtcagtagcaactacatgagctgggtccgccaggctccagggaaggggctggagtgggtctcacttatttatagtggtggtagcacatactacgcagactccgtgaagggcagattcaccatctccagagactattccaagaacacgctgtatcttcaaatgaacagcctgagagccgaggacacggctacgtattattgtgcgagagaacgtccccgcggtgcgggggagtactggggccagggaaccctggtcaccgtctcctcagcgtcgaccaagggcccatcggtcttcc

SEQ ID NO: 34 - DNA, 386 bp, synthetic construct
  misc_feature 1..386: note = Synthetic- 0089-IGK
  source 1..386: mol_type = other DNA
atcctttttctagtagcaactgcaaccggtgtacattctgacatccagatgacccagtctccatcgtccctgtctgcatctgtaggagacagagtcaccatcacttgccaggcgagtcaggacattaacatctatttaaattggtatcagcagaaaccaggaaaagcccctaagctcctgatctacgatgcatccaatttggaaacaggggtcccatcaaggttcagtggaagtggatctgggacagattttactttcaccatcagcagcctgcagcctgaagatattgcaacatattactgtcatcagcatgataatctccctcggacttttggccaggggaccaaggtggaaatcaaacgtacggtggctgcaccatctgtctt

SEQ ID NO: 35 - DNA, 421 bp, synthetic construct
  misc_feature 1..421: note = Synthetic- 0092-IGG1
  source 1..421: mol_type = other DNA
atcctttttctagtagcaactgcaaccggtgtacattccgaggtgcagctggtgcagtctggggctgaggtgcagaagcctggggcctcagtgaaggtttcctgcaaggcatctggacacaccttcaccagctattatatacactgggtgcgacaggcccctggacaagggcttgagtggatgggaatactcaacactagtggtggtagcacaacctacgcacagaagttccagggcagagtcaccatgaccagggacacgtccacgagcacagtctacatggacctgagcagcctgagatctgaggacacggccgtgtattactgtgcttcgtcttcgtgggatgatgcttttgatatctggggccaagggacaatggtcaccgtctcttcagcgtcgaccaagggcccatcggtcttcc

SEQ ID NO: 36 - DNA, 433 bp, synthetic construct
  misc_feature 1..433: note = Synthetic- 0093-IGL2
  source 1..433: mol_type = other DNA
atcctttttctagtagcaactgcaaccggttccaattcycagactgtggtgacycaggagccctcactgactgtgtccccaggagggacagtcactctcacctgtgcttccagcactggagcagtcaccagtggttactatccaaattggttccagcagaaacctggacaagcacccagggcactgatttatagtacaaggaacaaacattcctggacccctgcccggttctcaggctccctccttgggggcagagctgccctgacactgtcaggtgtgcagcctgaggacgaggctgagtattactgcctgctctactatggtggtccttgggtgttcggcggagggaccaagctgaccgtcctaggtcagcccaaggctgccccctcggtcactctgttcccaccctcgagtgaggagcttcaagccaaca

SEQ ID NO: 37 - DNA, 445 bp, synthetic construct
  misc_feature 1..445: note = Synthetic- 0094-IGG1
  source 1..445: mol_type = other DNA
atcctttttctagtagcaactgcaaccggtgtacattccgaggtgcagctggtgcagtctggggctgaggtgaagaagcctggggcctcagtgaaggtttcctgcaaggcatctggatacaccttcatgaactattatatgcactgggtgcgacaggcccctggacaagggcttgagtggatgggaatgatcaacccgagtggtggtagcgcaacctacgcacagaagttccagggcagagtcaccatgaccagggacacgtccacgagcacagtttacatggagctgagcagcctgagatctgaggacacggccgtttattactgtgcgagagaggagagaggttgtagtactaccagctgctatgatgatgcttttgatatttggggccaagggacaatggtcaccgtctcttcagcgtcgaccaagggcccatcggtcttcc

SEQ ID NO: 38 - DNA, 386 bp, synthetic construct
  misc_feature 1..386: note = Synthetic- 0095-IGK
  source 1..386: mol_type = other DNA
atcctttttctagtagcaactgcaaccggtgtacattctgacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaacgtacggtggctgcaccatctgtctt

SEQ ID NO: 39 - DNA, 430 bp, synthetic construct
  misc_feature 1..430: note = Synthetic- 0096-IGG1
  source 1..430: mol_type = other DNA
atcctttttctagtagcaactgcaaccggtgtacattccgaggtgcagctggtgcagtctggggctgaggtgaagaagcctggggcctcagtgaaggtttcctgcaaggcatctggatacaccttcatcacctactatatacactggatgcgacaggcccctggacaagggcttgagtggatgggactaatcaacccgagtggtggtagcacaaacttcgcacagaacttccagggcagagtcaccatgaccagggacacgtccacgagcacagtccacatggagctgaccagcctgagatctgaggacacggccgtgtattactgtgcgagaggggactccgggtatagcagcagctggtgtgattactggggccagggaaccctggtcaccgtctcctcagcgtcgaccaagggcccatcggtcttcc

SEQ ID NO: 40 - DNA, 427 bp, synthetic construct
  misc_feature 1..427: note = Synthetic- 0097-IGL2
  source 1..427: mol_type = other DNA
atcctttttctagtagcaactgcaaccggttctgtgacctcctatgagctgacwcaggaccctgctgtgtctgtggccttgggacagacagtcaggatcacatgccaaggcgacagcctcagaagctattctgcaagctggtaccagcagaggccaggacaggcccctgtacttgtcatctatgctaaagacaaccggccctcagggatcccagtccgattctctggctccagctcaggaaccacagcttccttgaccatcactggggctcaggcggaagatgaggctgactattactgtagctcccgggacagcagtgatactgtgctattcggcggagggaccaagttgaccgtcctaagtcagcccaaggctgccccctcggtcactctgttcccaccctcgagtgaggagcttcaagccaaca

SEQ ID NO: 41 - DNA, 487 bp, synthetic construct
  misc_feature 1..487: note = Synthetic- 0100-IGG1
  source 1..487: mol_type = other DNA
atcctttttctagtagcaactgcaaccggtgtacattccgaggtgcagctggtgcaggtgtccagtcccaggtccagctggtgacagtctggggctgaggtgaagaagcctgggtcctcggtgaaggtctcctgcaaggcttctggaggcaccttcagctactatgctatcaactgggtgcgacaggcccctggacaagggcttgagtgcatgggaaggatcatccctttccttggtatagcaaactacacacagagattccagggcagagtcacgattaccgcggacaaatccacgagcacagcctacatggagctgcgcagcctgagatctgaggacacggccgtatatttctgtgcgagagaggggccttattactatgatagtagtggttactcgaaatccgactccgacggtatggacgtctggggccaagggaccacggtcaccgtctcctcagcgtcgaccaagggcccatcggtcttcc

SEQ ID NO: 42 - DNA, 386 bp, synthetic construct
  misc_feature 1..386: note = Synthetic- 0101-IGK
  source 1..386: mol_type = other DNA
atcctttttctagtagcaactgcaaccggtgtacattcagacatccagttgacccagtctccatcctccctgtctgcatctgtaggagacagagtcaccatcacttgccgggccagtcagggcattagcagttatttagcctggtatcagcaaaaaccagggaaagcccctaagctcctgctctatgcttcatccactttgccaagtggggtcccatcaaggttcagcggcagtggatctgggacagatttcactctcaccatcagcagcctgcagcctgaagattttgcaacttattactgtcaacagcttaatagttaccctcccacttttggccaggggaccaaggtggaaatcaaacgtacggtggctgcaccatctgtctt

SEQ ID NO: 43 - DNA, 445 bp, synthetic construct
  misc_feature 1..445: note = Synthetic- 0104-IGG3
  source 1..445: mol_type = other DNA
atcctttttctagtagcaactgcaaccggtgtacattctgaggtgcagctggtggagtctgggggaggtgtggtacggcctggggggtccctcagactctcctgtgcagcctctggattcacctatgatacttatgggatgagttgggtccgccaagctccagggaagggactggagtgggtctctggtattaattggaatggtggtaggtcaggttatgcagactctgtgaagggccgattcatcatcttcagagacaacgccaagaactccctgtatctgcaaatgaacagtctgagagtcgaggacacggccttatattactgtgcgagagcaagcgtgggatattgtgctagtagcaggtgctccaactggttcgacacctggggccagggaaccctggtcaccgtctcctcagcgtcgaccaagggcccatcggtcttcc

SEQ ID NO: 44 - DNA, 436 bp, synthetic construct
  misc_feature 1..436: note = Synthetic- 0105-IGL2
  source 1..436: mol_type = other DNA
atcctttttctagtagcaactgcaaccggttcctgggcccagtctgtgctgackcagccgccctcattgtctgcggccccaggacagaaggtcaccatctcctgctctggaagcagctccaacattgggaataattatgtatcttggtaccaacaactcccaggagcagcccccaaactcctcatttatgacaataataagcgaccctcagggatttctgaccgattctctggctccatgtctggcacgtcagccaccctgggcatcaccgggctccagactggggacgagggcgattattactgcggaacatgggatagcagcctgagtcttgtggtgttcggcggagggaccaaactgaccgtcctaggtcagcccaaggctgccccctcggtcactctgttcccaccctcgagtgaggagcttcaagccaaca

SEQ ID NO: 45 - DNA, 421 bp, synthetic construct
  misc_feature 1..421: note = Synthetic- 0106-IGG1
  source 1..421: mol_type = other DNA
atcctttttctagtagcaactgcaaccggtgtacattctgaggtgcagctggtggagtctgggggaggcttggtccagcctggggggtccctgagactctcctgtgtagcctctggaatcaccgtcagtgccaattacatgagctgggtccgccaggctccagggaaggggctggagtgggtctcagttatttatagcggtggtagtactttctacgcagactccgtgaagggcaggttcaccatctccagagacaattccaagaacactctgtatcttcaaatgaacaacctgagagccgacgacacggctgtgtattcctgcgcgagagatttcaggggggcaactgcttttgatatctggggccaagggacaatggtcaccgtctcttcagcgtcgaccaagggcccatcggtcttcc

SEQ ID NO: 46 - DNA, 436 bp, synthetic construct
  misc_feature 1..436: note = Synthetic- 0107-IGL1
  source 1..436: mol_type = other DNA
atcctttttctagtagcaactgcaaccggttcctgggcccagtctgccctgactcagcctgcctccgtgtctgggtctcctggacagtcgatcaccatctcctgcactgcaaccagcagtgacgttgatgattataactatgtctcctggtaccaacaacacccaggcaaagcccccaaactcctgatttatgatgtcaataatcggccctcaggggtttccaatcgcttctctggctccaagtctggcaacacggcctccctgaccatctctgggctccaggctgaggacgaggctgattattactgcagctcatatacaagtagcagcactggagtcttcggatctgggaccaaggtcaccgtcctaggtcagcccaaggccaaccccactgtcactctgttcccaccctcgagtgaggagcttcaagccaaca

SEQ ID NO: 47 - DNA, 439 bp, synthetic construct
  misc_feature 1..439: note = Synthetic- 0108-IGG1
  source 1..439: mol_type = other DNA
atcctttttctagtagcaactgcaaccggtgtacattctgaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtaccacatactacgcagactccgttaagggcagattcaccctctccagagacaattccaagaacacgctgtatcttcaaatgaacagcctgagagccgaggacacggctgtgtattactgtgcgaggggttcctatgatagtagtggtttggtgatgagtggtgcttttgatatctggggccaagggacaatggtcaccgtctcttcagcgtcgaccaagggcccatcggtcttcc

SEQ ID NO: 48 - DNA, 436 bp, synthetic construct
  misc_feature 1..436: note = Synthetic- 0109-IGL2
  source 1..436: mol_type = other DNA
atcctttttctagtagcaactgcaaccggttcctgggcccagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaattgggtgttcggcggagggaccaagctgaccgtcctaggtcagcccaaggctgccccctcggtcactctgttcccaccctcgagtgaggagcttcaagccaaca

SEQ ID NO: 49 - DNA, 442 bp, synthetic construct
  misc_feature 1..442: note = Synthetic- 0110-IGG1
  source 1..442: mol_type = other DNA
atcctttttctagtagcaactgcaaccggtgtacattctgaggtgcagctggtggagtctgggggaggcttggtccagcctggggggtccctgaaactctcctgtgcagcctctgggttcagcttcagtgactctgctatgcactgggtccgccaggcttccgggaaagggctggagtgggtcggccgtattagaagcaaacctaacaattacgcgacagcatatgctgcgtcggtgaaaggcaggttcaccatctccagagatgattcaaagaacacggcgtatctgcaaatgaacagcctgaaaaccgaggacacggccgtttattattgtacttcctctcctcaactggaactgtacgtggactacggtatggacgtctggggccaagggaccacggtcaccgtctcctcagcgtcgaccaagggcccatcggtcttcc

SEQ ID NO: 50 - DNA, 386 bp, synthetic construct
  misc_feature 1..386: note = Synthetic- 0111-IGK
  source 1..386: mol_type = other DNA
atcctttttctagtagcaactgcaaccggtgtacattctgacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcatctcgtttacaaagtggggtcccatcaaggttcagcggcagtggatctgggacagaattcactctcaccatcagcagcctgcagcctgatgattttgcaacttattactgcctacagtatgatacttacccgtggaccttcggccaagggaccaaggtggaaatcaaacgtacggtggctgcaccatctgtctt

SEQ ID NO: 51 - DNA, 424 bp, synthetic construct
  misc_feature 1..424: note = Synthetic- 0114-IGG1
  source 1..424: mol_type = other DNA
atcctttttctagtagcaactgcaaccggtgtacattcccaggtgcagctgcaggagtcgggcccaggactggtgaagccttcacagaccctgtccctcacctgcactgtctctggtggctccatcaatagtggtggttactactggagctggatccgccagcacccagggaagggcctggagtggattgggtacatccattacagtgggagcacctactacagcccgtccctcaagagtcgaattaccatatcagtagacacgtctaaggaccagttctccctgaagctgagctctgtgactgccgcggacacggccgtatattactgtgcgagagaaaatgactggagctggttcgacccctggggccagggaaccctggtcaccgtctcctcagcgtcgaccaagggcccatcggtcttcc

SEQ ID NO: 52 - DNA, 386 bp, synthetic construct
  misc_feature 1..386: note = Synthetic- 0115-IGK
  source 1..386: mol_type = other DNA
atcctttttctagtagcaactgcaaccggtgtacattcagaaattgtgttgacacagtctccagccaccctgtctttgtctccaggggaaagagccaccctctcctgcagggccagtcagagtgttaggagctacttagcctggtaccaacagaaacctggccaggctcccaggctcctcatctatgatgcatccaacagggccactggcatcccagccaggttcagtggcagtgggtctgggacagacttcactctcaccatcagcagtctagagcctgaagattttgcagtttattactgtcagcagcgtagcaactggcctaagacgttcggccaagggaccaaggtggaaatcaaacgtacggtggctgcaccatctgtctt

SEQ ID NO: 53 - DNA, 463 bp, synthetic construct
  misc_feature 1..463: note = Synthetic- 0116-IGG1
  source 1..463: mol_type = other DNA
atcctttttctagtagcaactgcaaccggtgtacattcccaggtgcagctacagcagtggggcgcaggactgttgaagccttcggaaatcctgtcccgcacctgcgctgtctttggtgggtccttaagcggttactcttggagctggatccgccaacccccagggaagggcctggagtggattggagaaatcacttatagtggaaacaccaggtacaacccgtccctcaagagtcgagtcaccgtgtcagtggacacgtccaagaatcagttctccctgaggctgagttctgtgaccgccgcggacacggctgtatatttctgtgcgagagttatgaatggagtagtaccatcccctctaggggggctgggtccatggtactcctacgacgctatggacgtctggggccaggggaccacggtcaccgtctcctcagcgtcgaccaagggcccatcggtcttcc

SEQ ID NO: 54 - DNA, 427 bp, synthetic construct
  misc_feature 1..427: note = Synthetic- 0117-IGL1
  source 1..427: mol_type = other DNA
atcctttttctagtagcaactgcaaccggttctgtgacctcctatgagctgacwcaggaccctgctgtgtctgtggccttgggacagacagtcaggatcacatgccaaggagacagtctcagaaaatattatgcaagttggtatcaacagaagccaggacaggcccctgtacttgtcatctacggtaaaaatagccggccctcagggatcccagaccgattctctggctccacctcaggagacacagcttccttggccatcgctgagactcaggcggaagacgaggctgaatactactgtcactcccgggacaacactggtgaccatgtcttcggaactgggaccaaggtcaccattctaggtcagcccaaggccaaccccactgtcactctgttcccaccctcgagtgaggagcttcaagccaaca

SEQ ID NO: 55 - DNA, 433 bp, synthetic construct
  misc_feature 1..433: note = Synthetic- 0118-IGG1
  source 1..433: mol_type = other DNA
atcctttttctagtagcaactgcaaccggtgtacattcccaggtgcagctgcaggagtcgggcccaggactgctgaagccttcggagaccctgtccctcagctgcactgtctctggtggctccatcagtagttactactggagctggatccggcagcccccagggaagggactggagtggattgggtatatctattacagtgggagcaccagttacaacccctccctcaagagtcgagtcgccatatcagtagacacgtccaagaaccagttctccctgaagctgagctctgtgaccgctgcggacacggccgtgtattactgtgcgaccgattactatgatagtagtggttactactacggtatggacgtctggggccaagggaccacggtcaccgtctcctcagcgtcgaccaagggcccatcggtcttcc

SEQ ID NO: 56 - DNA, 383 bp, synthetic construct
  misc_feature 1..383: note = Synthetic- 0119-IGK
  source 1..383: mol_type = other DNA
atcctttttctagtagcaactgcaaccggtgtacattctgacatccagatgacccagtctccatcttccgtgtctgcatctgttggagacagagtcaccatcacttgtcgggcgagtcaggatattggcagctggtcagcctggtatcagcagaaaccagggaaagcccctaagctcctgatctatgctgtatccaatttgcaaagtggggtcccatcaaggttcagcggcagtggatctgggacagatttcactctcaccatcagcggcctgcagcctgaagattttgcaacttactattgtcaacaggctaacagtttccggacgttcggccaagggaccaaggtggaaatcaaacgtacggtggctgcaccatctgtctt

SEQ ID NO: 57 - protein, 15 aa, synthetic construct
  REGION 1..15: note = Synthetic- ACE2 peptides detected in RBD-bound EV pellets
GDYEVNGVDGYDYSR

SEQ ID NO: 58 - protein, 12 aa, synthetic construct
  REGION 1..12: note = Synthetic- ACE2 peptides detected in RBD-bound EV pellets
NQMILFGEEDVR

SEQ ID NO: 59 - protein, 19 aa, synthetic construct
  REGION 1..19: note = Synthetic- ACE2 peptides detected in RBD-bound EV pellets
NSFVGWSTDWSPYADQSIK

SEQ ID NO: 60 - protein, 18 aa, synthetic construct
  REGION 1..18: note = Synthetic- ACE2 peptides detected in RBD-bound EV pellets
PNIDVTDAMVDQAWDAQR

SEQ ID NO: 61 - protein, 11 aa, synthetic construct
  REGION 1..11: note = Synthetic- ACE2 peptides detected in RBD-bound EV pellets
SMLTDPGNVQK

SEQ ID NO: 62 - protein, 12 aa, synthetic construct
  REGION 1..12: note = Synthetic- ACE2 peptides detected in evHEK-ACE2 (positive control)
ANHYEDYGDYWR

SEQ ID NO: 63 - protein, 13 aa, synthetic construct
  REGION 1..13: note = Synthetic- ACE2 peptides detected in evHEK-ACE2 (positive control)
AYEWNDNEMYLFR

SEQ ID NO: 64 - protein, 15 aa, synthetic construct
  REGION 1..15: note = Synthetic- ACE2 peptides detected in evHEK-ACE2 (positive control)
GQLIEDVEHTFEEIK

SEQ ID NO: 65 - protein, 7 aa, synthetic construct
  REGION 1..7: note = Synthetic- ACE2 peptides detected in evHEK-ACE2 (positive control)
HEGPLHK

SEQ ID NO: 66 - protein, 8 aa, synthetic construct
  REGION 1..8: note = Synthetic- ACE2 peptides detected in evHEK-ACE2 (positive control)
LWAWESWR

SEQ ID NO: 67 - protein, 19 aa, synthetic construct
  REGION 1..19: note = Synthetic- ACE2 peptides detected in evHEK-ACE2 (positive control)
NSFVGWSTDWSPYADQSIK

SEQ ID NO: 68 - protein, 14 aa, synthetic construct
  REGION 1..14: note = Synthetic- ACE2 peptides detected in evHEK-ACE2 (positive control)
PLLNYFEPLFTWLK

SEQ ID NO: 69 - protein, 11 aa, synthetic construct
  REGION 1..11: note = Synthetic- ACE2 peptides detected in evHEK-ACE2 (positive control)
PLYEHLHAYVR

SEQ ID NO: 70 - protein, 13 aa, synthetic construct
  REGION 1..13: note = Synthetic- ACE2 peptides detected in evHEK-ACE2 (positive control)
QLRPLYEEYVVLK

SEQ ID NO: 71 - protein, 17 aa, synthetic construct
  REGION 1..17: note = Synthetic- ACE2 peptides detected in evHEK-ACE2 (positive control)
RLNTILNTMSTIYSTGK

SEQ ID NO: 72 - DNA, 5746 bp, synthetic construct
  misc_feature 1..5746: note = Synthetic- vector AbVec-hIgG1
  source 1..5746: mol_type = other DNA
ttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaaccggtgtacactcgagcgtacggtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa